SEQUENCE LISTING (12 sequences)

SEQ ID NO: 1 - DNA, 20 bp, Artificial Sequence
  Synthetic
ggagcgggtggagtatctct

SEQ ID NO: 2 - DNA, 20 bp, Artificial Sequence
  Synthetic
gcaacttcacggtgctcaac

SEQ ID NO: 3 - DNA, 20 bp, Artificial Sequence
  Synthetic
ggaagctccactatcctcgc

SEQ ID NO: 4 - DNA, 20 bp, Artificial Sequence
  Synthetic
ctacttcgccaacctgagca

SEQ ID NO: 5 - DNA, 21 bp, Artificial Sequence
  Synthetic
ctccaccatcaagaaccacga

SEQ ID NO: 6 - DNA, 20 bp, Artificial Sequence
  Synthetic
ccctcacgagcaaagaggaa

SEQ ID NO: 7 - DNA, 20 bp, Artificial Sequence
  Synthetic
gaggtaagcgttggggtgaa

SEQ ID NO: 8 - DNA, 20 bp, Artificial Sequence
  Synthetic
tgaggtagaggtccagcgat

SEQ ID NO: 9 - DNA, 20 bp, Artificial Sequence
  Synthetic
cggcgatccttgtactcctc

SEQ ID NO: 10 - DNA, 20 bp, Artificial Sequence
  Synthetic
tcatgagcatgtagcgccag

SEQ ID NO: 11 - DNA, 20 bp, Artificial Sequence
  Synthetic
cgtgtggacgggtagttctg

SEQ ID NO: 12 - DNA, 20 bp, Artificial Sequence
  Synthetic
gcctggttgtgcaggtctat

(19) United States
(12) Patent Application Publication
(10) Pub. No.: US 2014/0241988 A1
(43) Pub. Date: Aug. 28, 2014

(54) METHODS AND COMPOSITIONS FOR TREATING ATRIAL FIBRILLATION

(71) Applicant: The Regents of The University of Michigan, Ann Arbor, MI (US)

(72) Inventor: José Jalife, Saline, MI (US)

(73) Assignee: The Regents of The University of Michigan, Ann Arbor, MI (US)

(21) Appl. No.: 14/170,113

(22) Filed: Jan. 31, 2014

Related U.S. Application Data
(60) Provisional application No. 61/758,933, filed on Jan. 31, 2013.
(60) Provisional application No. 61/860,593, filed on Jul. 31, 2013.

(51) Int. Cl. A61K 31/736; A61K 49/00; C07K 16/18; A61K 31/732; A61K 31/713

(52) U.S. Cl. CPC A61K 31/736; A61K 31/732; A61K 31/713; C07K 16/18; A61K 49/0004
USPC 424/9.2; 514/54; 514/44 A; 424/172.1

(57) ABSTRACT

The present invention relates to compositions and methods for the prevention and treatment of atrial fibrillation. In particular, the present invention provides therapeutic agents for the treatment and prevention of persistent and permanent atrial fibrillation and prevention of progression of atrial fibrillation to permanent atrial fibrillation.

### CROSS REFERENCE TO RELATED APPLICATIONS

This application claims priority to U.S. Provisional Patent Application No. 61/758,933, filed Jan. 31, 2013, and U.S. Provisional Patent Application No. 61/860,593, filed Jul. 31, 2013, each of which are herein incorporated by reference in its entirety.

### FIELD OF THE INVENTION

The present invention relates to compositions and methods for the prevention and treatment of atrial fibrillation. In particular, the present invention provides therapeutic agents for the treatment and prevention of persistent and permanent atrial fibrillation and prevention of progression of atrial fibrillation to permanent atrial fibrillation.

### BACKGROUND OF THE INVENTION

Atrial fibrillation (AF or A-fib) is the most common cardiac arrhythmia (irregular heart beat). It may cause no symptoms, but it is often associated with palpitations, fainting, chest pain, or congestive heart failure.

AF increases the risk of stroke; the degree of stroke risk can be up to seven times that of the average population, depending on the presence of additional risk factors (such as high blood pressure). It may be identified clinically when taking a pulse, and the presence of AF can be confirmed with an electrocardiogram (ECG or EKG) which demonstrates the absence of P waves together with an irregular ventricular rate.

In AF, the normal regular electrical impulses generated by the sinoatrial node are overwhelmed by disorganized electrical impulses usually originating in the posterior wall of the left atrium near the roots of the pulmonary veins, leading to irregular conduction of impulses to the ventricles which generate the heartbeat. AF may occur in episodes lasting from minutes to a maximum of seven days (“paroxysmal”), or it may be persistent or permanent in nature. A number of medical conditions increase the risk of AF, particularly mitral stenosis (narrowing of the mitral valve of the heart).

Atrial fibrillation may be treated with medications to either slow the heart rate to a normal range (“rate control”) or revert the heart rhythm back to normal (“rhythm control”). Synchronized electrical cardioversion can be used to convert AF to a normal heart rhythm. Surgical and catheter-based therapies may be used to prevent recurrence of AF in certain individuals. People with AF often take anticoagulants such as warfarin to protect them from stroke, depending on the calculated risk. The prevalence of AF in a population increases with age, with 8% of people over 80 having AF. Chronic AF leads to a small increase in the risk of death. A third of all strokes are caused by AF.

Anticoagulation can be achieved through a number of means including the use of aspirin, heparin, warfarin, and dabigatran. Which method is used depends on a number issues including: cost, risk of stroke, risk of falls, compliance, and speed of desired onset of anticoagulation. Rate control is achieved with medications that work by increasing the degree of block at the level of the AV node, effectively decreasing the number of impulses that conduct into the ventricles. This can be done with: beta blockers (preferably the “cardioselective” beta blockers such as metoprolol, atenolol, bisoprolol, nebivolol), non-dihydropyridine calcium channel blockers (i.e. diltiazem or verapamil).

Cardioversion is a noninvasive conversion of an irregular heartbeat to a normal heartbeat using electrical or chemical means. Electrical cardioversion involves the restoration of normal heart rhythm through the application of a DC electrical shock. Chemical cardioversion is performed with drugs, such as amiodarone, dronedarone, procainamide, dofetilide, ibutilide, propafenone, or flecainide.

### SUMMARY

The present invention relates to compositions and methods for the prevention and treatment of atrial fibrillation. In particular, the present invention provides therapeutic agents for the treatment and prevention of persistent and permanent atrial fibrillation and prevention of progression of atrial fibrillation to permanent atrial fibrillation.

Embodiments of the present invention provide a method of treating atrial fibrillation (e.g., persistent or paroxysmal AF) or preventing persistent or permanent atrial fibrillation (e.g., preventing progression from paroxysmal to permanent AF) in a subject, comprising: administering an agent that inhibits at least one activity of a galectin polypeptide to the subject, wherein the administering treats or prevents atrial fibrillation in the subject. In some embodiments, the agent is, for example, a small molecule, a natural product (e.g., plant-based natural product such as a carbohydrate. Examples include, but are not limited to, pectin, pectin fragment, or derivative thereof (e.g., citrus pectin), ranolazine (See e.g., U.S. Pat. No. 4,567,264; herein incorporated by reference in its entirety), GM-CT-01, GR-MD-02, N-acetyllactosamine (N-Lac) (Sigma-Aldrich, St. Louis, Mo.),

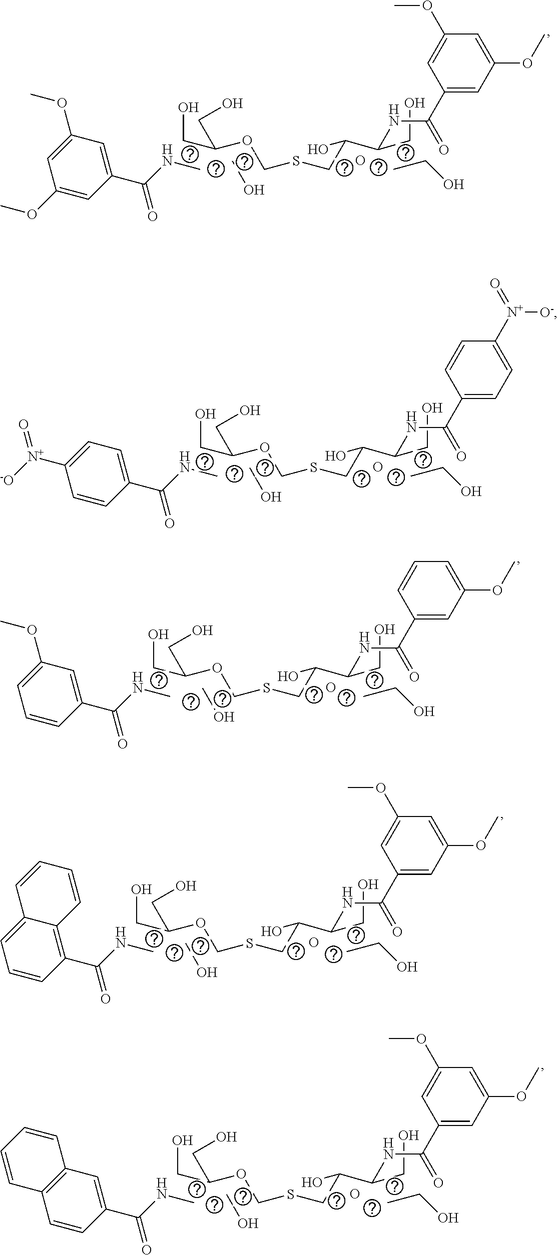

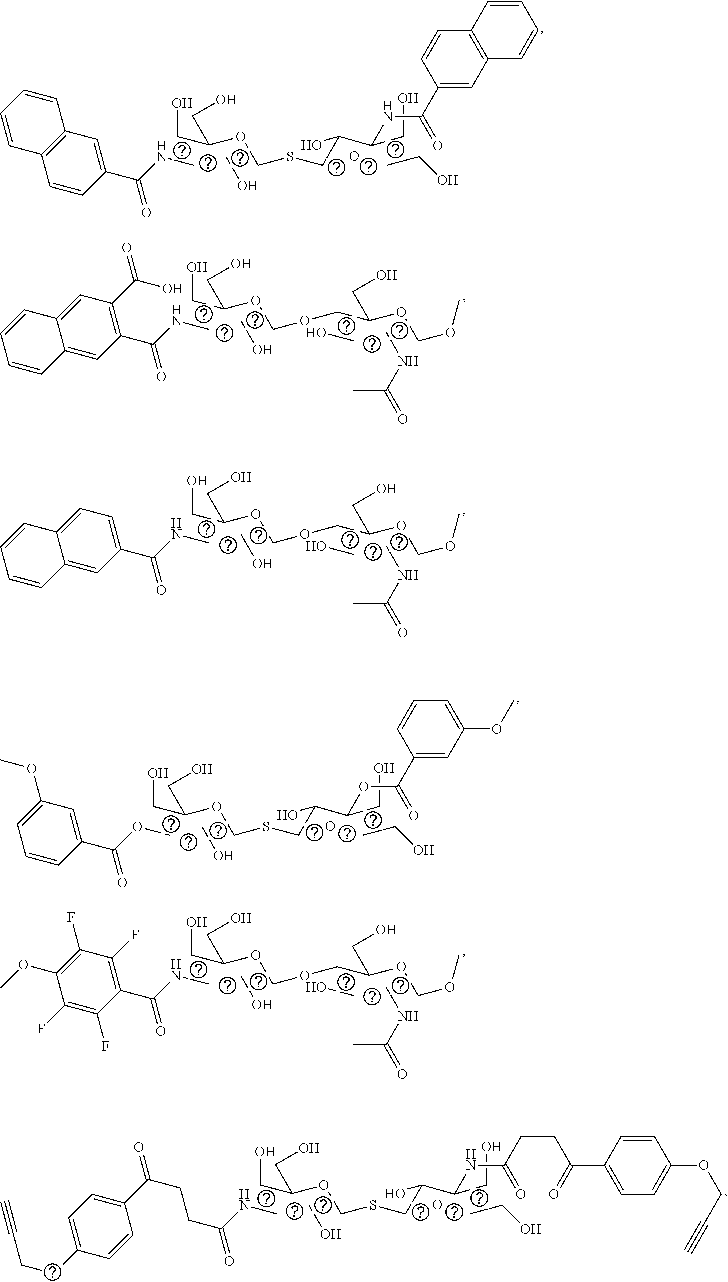

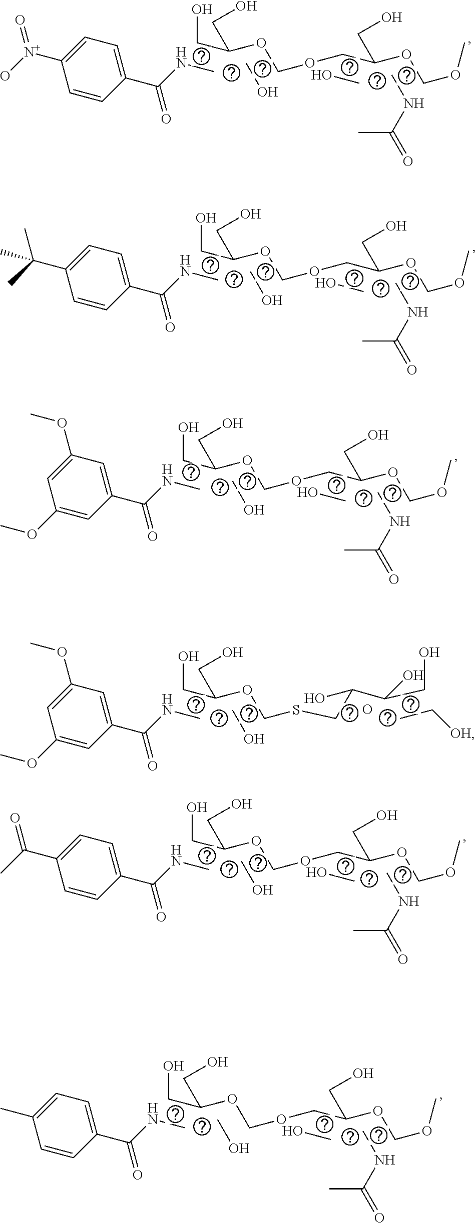

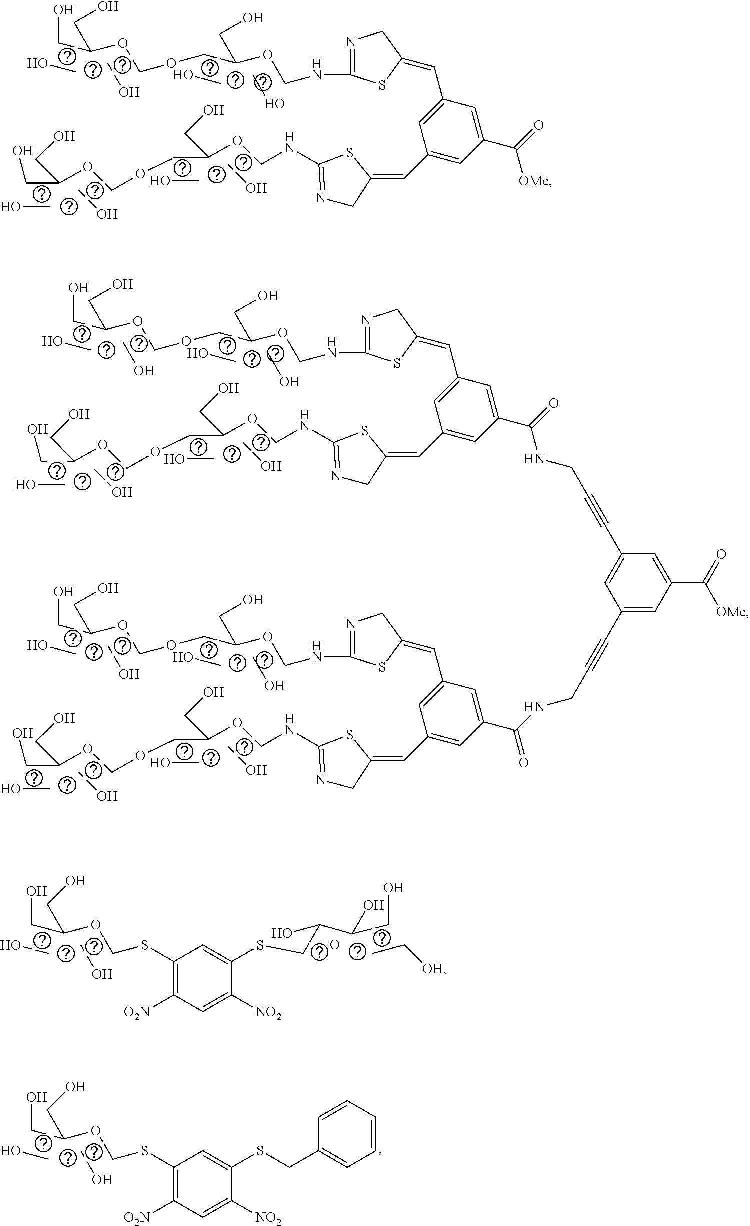

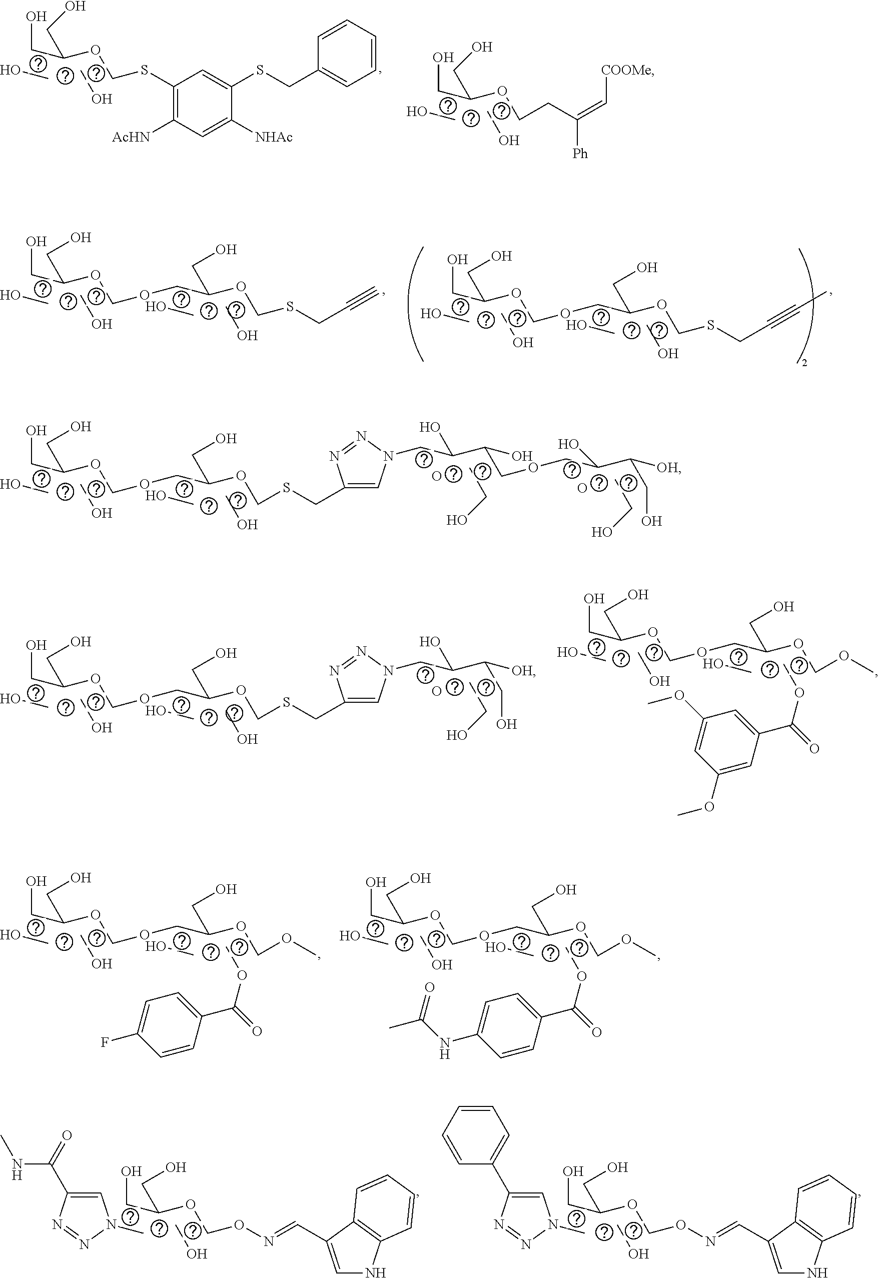

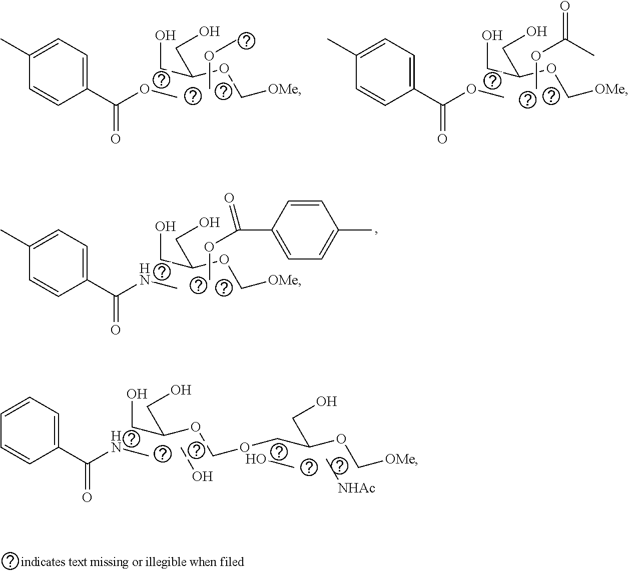

an siRNA, or an antibody. In some embodiments, the galectin polypeptide is galectin-2 or galectin-3 (e.g., galectin-3). In some embodiments, the subject has been diagnosed with atrial fibrillation (e.g., paroxysmal AF) or is at risk of atrial fibrillation (e.g. persistent AF). In some embodiments, the subject has had at least one prior incident of atrial fibrillation and the administering prevents future incidents of atrial fibrillation in the subject. In some embodiments, the subject is a human or a non-human mammal. In some embodiments, the subject does not have a fibrotic disease (e.g., liver fibrosis) and/or cancer (e.g., colorectal cancer or melanoma).

In some embodiments, the present invention provides a method of treating atrial fibrillation or preventing persistent atrial fibrillation in a subject, comprising: administering GM-CT-01 or GR-MD-02 to the subject, wherein the administering treats or prevents atrial fibrillation in said subject.

In some embodiments, the present invention provides a method of treating atrial fibrillation or preventing persistent atrial fibrillation in a subject, comprising: administering GM-CT-01 to the subject, wherein the administering treats or prevents atrial fibrillation in said subject

The present invention additionally provides the use of an agent that inhibits at least one biological activity of a galectin polypeptide in the treatment or prevention of atrial fibrillation (e.g., prevention of progression from early or paroxysmal AF to persistent or permanent AF).

Embodiments of the present invention provide the use of GM-CT-01 or GR-MD-02 in the treatment of atrial fibrillation or prevention of persistent atrial fibrillation.

Further embodiments provide the use of GM-CT-01 in the treatment of atrial fibrillation or prevention of persistent atrial fibrillation.

The present invention further provides nutritional supplements, food additives, food products, or foods comprising at least one agent that inhibits at least one biological activity of a galectin polypeptide (e.g., for in the treatment or prevention of atrial fibrillation).

In some embodiments, the present invention provides a method of screening compounds, comprising: a) administering a test compound to a non-human animal (e.g., ovine) that exhibits a transition from paroxysmal to long-standing persistent atrial fibrillation; and b) identifying compounds that inhibit or delay the transition. In some embodiments, the paroxysmal and self-sustained AF is induced by atrial tachypacing (e.g., burst tachypacing). In some embodiments, the test compound is administered prior to a first episode of atrial fibrillation. In some embodiments, the test compound is administered repeatedly.

Additional embodiments are described herein.

### BRIEF DESCRIPTION OF THE DRAWINGS

FIG. 1 shows Gal-3 protein expression in adult sheep atrial fibroblasts. FM, full media; SFM, serum free media. Left, representative western blot image. Right, quantification of Gal-3 relative expression.

FIG. 2 demonstrates immunolocalization experiments showing that Gal-3 co-localizes with α-SMA in stress fibers of adult sheep atrial fibroblasts cultured in full media (DMEM with 10% FBS) for 48 hours.

FIG. 3 shows that Gal-3 increases the rate of wound healing. A, sequential micrographs (days 0-2) show the evolution of the wound in SFM (top) and 10 μg/ml Gal-3. B, bar graph compares wound widths at days 0-2 in SFM; Gal-3 1 μg/ml; Gal-3 10 μg/ml; and FM.

FIG. 4 shows in FIG. 4A that Gal-3 (G3, 1-30 μg/ml) increases proliferation of sheep atrial myofibroblasts and that in the presence of two different concentrations (0.125 and 0.6 mg/ml) of Gla-3 inhibitor (GM-CT-01), Gal-3 failed to increase sheep atrial myofibroblast proliferation. FIG. 4B shows that the increase in fibroblast proliferation induced by galectin-3 is prevented by pre-treatment with neutralizing TGF-β1 antibody.

FIG. 5 shows structural remodeling in the sheep model of persistent AF. A. Western blots from control (C) and persistent AF (P) sheep atria showing increase in α-smooth muscle actin (SMA) expression after 2 months of tachypacing. B. Quantification of aSMA relative to GAPDH. C. Increase in interstitial fibrosis in the left atrial appendage (LAA), right atrial appendage (RAA) and left pulmonary vein (LPV) in control versus 2 months tachypacing.

FIG. 6 shows upregulation of TGF-β1 and Gal-3 in left atrial appendage (LAA) of sheep with persistent AF.

FIG. 7 shows a model of the galectin signaling pathway.

FIG. 8 shows effects of fibroblast conditioned medium (FCM) on peak inward sodium current (INa) after 72 hr of treatment. Representative current traces A) Top Panel, control; Lower Panel, FCM. Current-voltage relationships for control, FCM and FCM+TGF-β1 antibody (TGF-β1 ab) is shown in panel B. C) Time dependent recovery of the channel. D) Voltage dependence of inactivation (h∞ curve) and Voltage dependence of activation (m∞ curve).

FIG. 9 shows effects of fibroblast conditioned medium (FCM) on outward potassium current (Ito) after 72 hr treatment. Representative current traces A) control, B) FCM. Ito IV relation at voltages between −40 and +60 mV for control, FCM and FCM+TGF-β1 ab is shown in panel C. D) peak Ito under control conditions.

FIG. 10 shows protein analyses of cytokine expression in fibroblast conditioned media. A) Protein analyses of cytokine expression in fibroblast conditioned media by cytokine array. Values are mean±standard error of the mean, N=3 in each group. B) Concentration of TGF-β1 in rat myocyte conditioned medium (black) and FCM (white) determined using a rat TGF-β1 ELISA kit (R&D Systems).

FIG. 11 shows effects of TGF-β1 on peak inward sodium current (INa) after 72 hr of treatment. Representative current traces A) control, B) TGF-β1. C). Dose response curve. Data are from 61 cells, 24 hearts. D) Current-voltage relationships for control, TGF-β1 and TGF-β1+TGF-β1 antibody (AB). Shown in panel E, Voltage dependence of activation (m∞ curve) and inactivation (h∞ curve). F) Time dependent recovery of the channel.

FIG. 12 shows effects of TGF-β1 on outward potassium current (Ito) after 72 hr treatment. Representative current traces A) control, B) TGF-β1, Ito IV relation at voltages between −40 and +60 mV for control, TGF-β1 and TGF-β1+TGF-β1 antibody (AB) is shown in panel C. D) peak Ito under control conditions. Values are mean±standard error. N=8-18 cells from 5 different isolations. * indicates p<0.05.

FIG. 13 shows effects of TGF-β1 on the action potential duration (APD) of ventricular myocytes at 72 hours. A) representative APs in control and TGF-β1 (1-50 ng/ml at BCL=1000 ms. B-D), Cycle length dependent changes in APD30, APD50, APD90 in control, TGF-β1 1 ng/ml and TGF-β1 10 ng/ml.

FIG. 14 shows effects of TGF-β1 on mRNA expression after 72 hr treatment with TGF-β1 (1 ng/ml). Real time PCR was performed using Taqman Primers. GAPDH was used as an internal control. A) Changes in SNC5A gene expression, B) changes in KCNIP2 gene expression and C) changes in KCND2 gene expression.

FIG. 15 shows effect of TGF-β1 treatment on FOXO1 phosphorylation in adult cardiac myocytes. A) Representative western blot image of FOXO phosphorylation by TGF-β1 B) Quantitative data from 6 different experiments. C) Effect of LY29004 (PI3K inhibitor) treatment on SCN5A expression in TGF-β1 treated cells.

FIG. 16 shows over expression of constitutive active FOXO1 in adult rat cardiac myocytes. A) Adult rat cardiac myocytes infected with 10 MOI of virus images taken at different time after infection. B) Representative image of western blot of adult rat cardiac myocytes lysate 72 hr after infection showing increased expression of GFP tagged FOXO-CA protein.

FIG. 17 shows a time-course of AF development. A: representative 3D plot of percentage of AF episodes in a given week (Y-axis) vs episode duration (X-axis) and weeks of follow-up after initiation of pacing (Z-axis). B: summary of temporal measurements.

FIG. 18 shows AF-induced changes in extracellular matrix. A: Mean+/−SEM values for patchy fibrosis (left) and interstitial fibrosis (right) in right atrium (RA), left atrium (LA) and posterior left atrium (PLA) of sham-operated (N=6), transition (N=7) and LS-PAF (N=7). B: Representative picrosirius red staining of PLA of sham-operated, transition and LS-PAF. C and D: Western blots of α-smooth muscle actin (SMA) and Collagen III (Col III) in LA and RA tissue homogenates relative to GAPDH.

FIG. 19 shows dominant frequency increases in RA and LA (A) and surface ECG (B) during progression of AF. N=14 for RA, N=8 for LA. #p<0.001 for RA vs. LA, **p<0.001 vs. sham.

FIG. 20 shows rate of increase in DF during paroxysmal AF predicts transition to persistent AF. A: Representative graphs for three animals. Left, sheep with the highest dDF/dt (0.14 Hz/day, time to transition 19 days); middle, intermediate dDF/dt (0.03 Hz/day, time to transition 46 days); right, lowest dDF/dt (0.003 Hz/day, time to transition 346 days); left and right from transition group, middle from LS-PAF group. B: log-log plots of time from first episode to onset of self-sustained persistent AF versus dDF/dt for the RA (intracardiac electrode), LA (loop recorder) and ECG (surface Lead 1).

FIG. 21 shows APD and frequency dependence in myocytes from sham, transition, and persistent AF. A: Action potential duration (APD90 at 1 Hz) is reduced in both atria at transition from paroxysmal to persistent AF. B: Cycle length (CL) dependence of APD90.

FIG. 22 shows sustained AF reduces functional expression of Na+ and L-type Ca2+ channels. A: Current-voltage relationships for INa in myocytes from LA (left) and RA (right). B: Current-voltage relationships for ICaL in myocytes from LA (left) and RA (right). C: Representative traces for INa (upper) and ICaL (lower) in myocytes from LA of sham-operated and LS-PAF animal. D-E: Western blot analysis of NaV1.5 and CaV1.2 protein expression in LA tissue homogenates (D) and RA tissue homogenates (E). Top, Representative blots; bottom, Quantification of protein expression relative to GAPDH. N=6. F-G: Real time RT-PCR analysis of SNC5A and CACNA1C gene expression in tissue homogenates from LA (F) and RA (G); quantification of gene expression relative to GAPDH. N=6.

FIG. 23 shows sustained AF increases functional expression of Kir2.3. A: Current-voltage relationships for IK1 in myocytes from LA (top) and RA (bottom). B: Western blots for Kir2.3 in LA tissue homogenates.

FIG. 24 shows simulations that predict consequences of ion channel remodeling on rotor frequency. A: Action potential traces for sham, paroxysmal and transition AF predicted by experimentally derived ion channel changes. B: Rotor in paroxysmal (left) had lower frequency than transition AF. C: Rotors in paroxysmal AF meandered considerably and eventually self-terminated upon collision with boundary.

FIG. 25 shows experimental and pacing protocols. A: Fluoroscopy image showing the RA lead screwed to the right atrial (RA) appendage (arrowhead) and the implantable loop recorder (ILR, black arrow) fixed subcutaneously in close proximity to the left atrium (LA). B: Top: A 30-second burst of tachypacing (20 Hz) during sinus rhythm (SR) induces a short-lasting episode of AF; bottom: intracardiac electrogram recorded from the RA showing AF termination (left), detection of SR by the automatic mode switching (AMS) algorithm and automatic resumption of pacing.

FIG. 26 shows that A: GMCT-01 prevented increase of DF from RA in vivo during AF progression. B: GMCT-01 prevented the increase of left atrial endo-diastolic volume (EDV) adjusted by body weight (BW) during AF progression. C: In optical mapping, APD90 were longer in GMCT-01 group. N=5 for SALINE, N=5 for GMCT-01 group.

FIG. 27 shows a protocol for a Gal-3 inhibitor trial in the ovine model of embodiments of the present disclosure.

FIG. 28 shows that Gal-3 inhibition lessens AF-induced atrial dilatation.

FIG. 29 shows that Gal-3 inhibition reduces mitral regurgitation (MR).

FIG. 30 shows that Gal-3 inhibition reduces Fibrosis in the PLA.

FIG. 31 shows that Gal-3 inhibition prevents the sustained AF increase in dominant frequency as measured in both RA and LA.

FIG. 32 shows that Gal-3 inhibition prevents the sustained AF-induced shortening of action potential duration in both RA and LA.

FIG. 33 shows that Gal-3 Inhibition increases the percentage of spontaneous terminations of persistent AF during treatment.

FIG. 34 shows that Gal-3 inhibition does not alter left ventricular function.

FIG. 35 shows echocardiographic evidence of sustained AF-induced atrial dilatation. Parasternal long-axis view of the heart of a sham-operated (A) and a long-standing persistent AF animal (B).

FIG. 36 shows quantification of echocardiographic findings. A: Left ventricular ejection fraction (LVEF) did not change over the time of the study. B and C: Both atria were significantly dilated in the LS-persistent AF animals. D: Mitral valve regurgitation, measured in arbitrary units (AU) of severity, where 1 is mild and 4 is severe, was significant in LS-persistent AF animals.

FIG. 37 shows that after the heart was explanted, the atria were removed and cut in the following three sections: RA wall (panel A), PLA (panel B) and LA wall (panel C).

FIG. 38 shows sustained AF induces atrial myocyte hypertrophy. A: Average lengths and widths for cells isolated from RA (open symbols) and LA (filled symbols). N=3/n=60 (sham), N=4/n=70 (transition), and N=5/n=90 (LS-PAF). B: Representative phase contrast micrographs.

FIG. 39 shows that sustained AF increases serum levels of Procollagen III N-Terminal Propeptide (PIIINP).

FIG. 40 shows the relationship between DF and time to transition to persistent AF.

FIG. 41 shows correlations between dDF/dt measured from the signal obtained through the RA intracardiac lead, the ILR and the ECG (lead I).

FIG. 42 shows calcium handling protein changes. Western blot analysis of SERCA (panel A), phospholamban (Panel B), Sodium-calcium exchanger (NCX, panel C) and CaMKII (panel D). Left: representative blots; right: quantification of protein expression relative to GAPDH.

FIG. 43 shows ryanodine receptor (RyR2) changes.

FIG. 44 shows that sustained AF reduces functional expression of the transient outward potassium channel (Ito) but not hERG. A and B: Current-voltage relationships for Ito in cells from the LA (A) and the RA (B). For the LA: N=2/n=6 (sham), N=3/n=5 (transition), N=6/n=10 (LS-PAF); for the RA: N=2/n=6 (sham), N=3/n=5 (transition), N=3/n=5 (LS-PAF). *p<0.05 vs. sham for the transition and LS-PAF groups. C and D: Western blot analysis of KV4.2 and KV 11.1 protein expression in LA (C) and RA (D) tissue homogenates. Top, Representative blots; bottom, Quantification of protein expression relative to GAPDH.

FIG. 45 shows effects of increasing IK1 alone in the Grandi-Pandit human atrial model. A: Increasing IK1 by 100% alone as seen in myocytes from transition sheep hyperpolarized the resting membrane potential by −2 mV and significantly shortened the APD (23%) with respect to sham. B: IK1 increase alone resulted in a meandering rotor at 4.7 Hz.

FIG. 46 shows effects of reducing ICaL alone in the Grandi-Pandit human atrial model. A: When ICaL was reduced by 30%, as simulated in paroxysmal AF, APD50 and APD90 were reduced (˜37%), which resulted in a meandering rotor that eventually died out. B: When ICaL was reduced by 65%, as observed in transition AF, APD50 and APD90 were greatly reduced (˜64%).

FIG. 47 shows effects of reducing Ito alone in the Grandi-Pandit human atrial model. A. reducing Ito by 75% resulted in only slight increases in APD30 and APD50. B: This condition yielded a meandering and unstable rotor whose DF was 3.38 Hz

FIG. 48 shows the effects of reducing INa alone in the Grandi-Pandit human atrial model. A. reducing INa by 50% negligibly changed APD90. B: this condition resulted in an unstable rotor whose DF was 3.82 Hz.

FIG. 49 shows electrophysiological differences between fast and slow transition animals. A. dDF/dt was significantly higher in fast transition sheep (0.07±0.02 Hz/day; N=7) than slow transition sheep (0.02±0.007 Hz/day; N=7; **p=0.007; *p=0.036). B, Mean APD at 30-90% repolarization was shorter in fast than slow transition animals. C, ICaL tended to be lower in fast than slow transition animals; top, LA bottom, RA. D, IK1 tended to be larger in slow transition animals. N=4, fast transition; N=3 slow transition sheep. N=number of animals.

FIG. 50 shows structural differences between fast and slow transition animals. LA area was significantly increased in both groups (**p=0.007; *p<0.05), although a more pronounced atrial dilatation was observed in slow transition animals (124% vs. 45% increase in LA atrial dilatation; p=0.014, panel A). Trends for higher degree of fibrosis (panel B), longer and wider cells (panel C) and heavier atria (panel D) were observed. N=4 fast transition; N=3 slow transition sheep. N=number of animals.

### DEFINITIONS

To facilitate an understanding of the present invention, a number of terms and phrases are defined below.

As used herein, the term “inhibits at least one biological activity of Galectin” refers to any agent that decreases any activity of a galectin polypeptide (e.g., Gal-3) (e.g., including, but not limited to, the activities described herein), via directly contacting galectin protein, contacting galectin mRNA or genomic DNA, causing conformational changes of galectin polypeptides, decreasing galectin protein levels, or interfering with galectin interactions with signaling partners, and affecting the expression of galectin target genes Inhibitors also include molecules that indirectly regulate galectin biological activity by intercepting or otherwise influencing upstream or downstream signaling molecules.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., AF). Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present invention.

The term “chemical moiety” refers to any chemical compound containing at least one carbon atom. Examples of chemical moieties include, but are not limited to, aromatic chemical moieties, chemical moieties comprising sulfur, chemical moieties comprising nitrogen, hydrophilic chemical moieties, and hydrophobic chemical moieties As used herein, the term “aliphatic” represents the groups including, but not limited to, alkyl, alkenyl, alkynyl, alicyclic.

As used herein, the term “alkyl” refers to an unsaturated carbon chain substituent group. In general, alkyls have the general formula CₙH₂ₙ₊₁. Exemplary alkyls include, but are not limited to, methyl (CH₃), ethyl (C₂H₅), propyl (C₃H₇), butyl (C₄H₉), pentyl (C₅H₁₁), etc.

As used herein, the term “aryl” represents a single aromatic ring such as a phenyl ring, or two or more aromatic rings (e.g., bisphenyl, naphthalene, anthracene), or an aromatic ring and one or more non-aromatic rings. The aryl group can be optionally substituted with a lower aliphatic group (e.g., alkyl, alkenyl, alkynyl, or alicyclic). Additionally, the aliphatic and aryl groups can be further substituted by one or more functional groups including, but not limited to, chemical moieties comprising N, S, O, —NH₂, —NHCOCH₃, —OH, lower alkoxy (C₁-C₄), and halo (—F, —Cl, —Br, or —I).

As used herein, the term “substituted aliphatic” refers to an alkane, alkene, alkyne, or alicyclic moiety where at least one of the aliphatic hydrogen atoms has been replaced by, for example, a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic, etc.). Examples of such include, but are not limited to, 1-chloroethyl and the like.

As used herein, the term “substituted aryl” refers to an aromatic ring or fused aromatic ring system consisting of at least one aromatic ring, and where at least one of the hydrogen atoms on a ring carbon has been replaced by, for example, a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, hydroxyphenyl and the like.

As used herein, the term “cycloaliphatic” refers to an aliphatic structure containing a fused ring system. Examples of such include, but are not limited to, decalin and the like.

As used herein, the term “substituted cycloaliphatic” refers to a cycloaliphatic structure where at least one of the aliphatic hydrogen atoms has been replaced by a halogen, a nitro, a thio, an amino, a hydroxy, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, 1-chlorodecalyl, bicyclo-heptanes, octanes, and nonanes (e.g., nonrbornyl) and the like.

As used herein, the term “heterocyclic” represents, for example, an aromatic or nonaromatic ring containing one or more heteroatoms. The heteroatoms can be the same or different from each other. Examples of heteroatoms include, but are not limited to nitrogen, oxygen and sulfur. Aromatic and nonaromatic heterocyclic rings are well-known in the art. Some nonlimiting examples of aromatic heterocyclic rings include pyridine, pyrimidine, indole, purine, quinoline and isoquinoline. Nonlimiting examples of nonaromatic heterocyclic compounds include piperidine, piperazine, morpholine, pyrrolidine and pyrazolidine. Examples of oxygen containing heterocyclic rings include, but not limited to furan, oxirane, 2H-pyran, 4H-pyran, 2H-chromene, and benzofuran. Examples of sulfur-containing heterocyclic rings include, but are not limited to, thiophene, benzothiophene, and parathiazine. Examples of nitrogen containing rings include, but not limited to, pyrrole, pyrrolidine, pyrazole, pyrazolidine, imidazole, imidazoline, imidazolidine, pyridine, piperidine, pyrazine, piperazine, pyrimidine, indole, purine, benzimidazole, quinoline, isoquinoline, triazole, and triazine. Examples of heterocyclic rings containing two different heteroatoms include, but are not limited to, phenothiazine, morpholine, parathiazine, oxazine, oxazole, thiazine, and thiazole. The heterocyclic ring is optionally further substituted with one or more groups selected from aliphatic, nitro, acetyl (i.e., —C(═O)—CH₃), or aryl groups.

As used herein, the term “substituted heterocyclic” refers to a heterocylic structure where at least one of the ring carbon atoms is replaced by oxygen, nitrogen or sulfur, and where at least one of the aliphatic hydrogen atoms has been replaced by a halogen, hydroxy, a thio, nitro, an amino, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to 2-chloropyranyl.

As used herein, the term “electron-rich heterocycle,” means cyclic compounds in which one or more ring atoms is a heteroatom (e.g., oxygen, nitrogen or sulfur), and the heteroatom has unpaired electrons which contribute to a 6-π electronic system. Exemplary electron-rich heterocycles include, but are not limited to, pyrrole, indole, furan, benzofuran, thiophene, benzothiophene and other similar structures.

As used herein, the term “lower-alkyl-substituted-amino” refers to any alkyl unit containing up to and including eight carbon atoms where one of the aliphatic hydrogen atoms is replaced by an amino group. Examples of such include, but are not limited to, ethylamino and the like.

As used herein, the term “lower-alkyl-substituted-halogen” refers to any alkyl chain containing up to and including eight carbon atoms where one of the aliphatic hydrogen atoms is replaced by a halogen. Examples of such include, but are not limited to, chlorethyl and the like.

As used herein, the term “acetylamino” shall mean any primary or secondary amino that is acetylated. Examples of such include, but are not limited to, acetamide and the like.

As used herein, the term “a moiety that participates in hydrogen bonding” as used herein represents a group that can accept or donate a proton to form a hydrogen bond thereby. Some specific non-limiting examples of moieties that participate in hydrogen bonding include a fluoro, oxygen-containing and nitrogen-containing groups that are well-known in the art. Some examples of oxygen-containing groups that participate in hydrogen bonding include: hydroxy, lower alkoxy, lower carbonyl, lower carboxyl, lower ethers and phenolic groups. The qualifier “lower” as used herein refers to lower aliphatic groups (C₁-C₄) to which the respective oxygen-containing functional group is attached. Thus, for example, the term “lower carbonyl” refers to inter alia, formaldehyde, acetaldehyde. Some nonlimiting examples of nitrogen-containing groups that participate in hydrogen bond formation include amino and amido groups. Additionally, groups containing both an oxygen and a nitrogen atom can also participate in hydrogen bond formation. Examples of such groups include nitro, N-hydroxy and nitrous groups. It is also possible that the hydrogen-bond acceptor in the present invention can be the electrons of an aromatic ring.

The term “derivative” of a compound, as used herein, refers to a chemically modified compound wherein the chemical modification takes place either at a functional group of the compound or backbone.

The term “diagnosed,” as used herein, refers to the recognition of a disease by its signs and symptoms (e.g., resistance to conventional therapies), or genetic analysis, pathological analysis, histological analysis, and the like.

As used herein, the term “host cell” refers to any eukaryotic or prokaryotic cell (e.g., mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “effective amount” refers to the amount of a compound (e.g., a compound of the present invention) sufficient to effect beneficial or desired results. An effective amount can be administered in one or more administrations, applications or dosages and is not limited intended to be limited to a particular formulation or administration route.

As used herein, the term “co-administration” refers to the administration of at least two agent(s) (e.g., a compound of the present invention) or therapies to a subject. In some embodiments, the co-administration of two or more agents/therapies is concurrent. In some embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “toxic” refers to any detrimental or harmful effects on a cell or tissue as compared to the same cell or tissue prior to the administration of the toxicant.

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

### DETAILED DESCRIPTION OF THE INVENTION

The present invention relates to compositions and methods for the prevention and treatment of atrial fibrillation. In particular, the present invention provides therapeutic agents for the treatment and prevention of persistent and permanent atrial fibrillation and prevention of progression of atrial fibrillation to permanent atrial fibrillation.

### I. Inhibitors

In some embodiments, the present invention provides compositions and methods for targeting galectin (e.g., Gal-3 or Gal-2) in order to treat atrial fibrillation or prevent persistent or permanent AF. Exemplary therapeutic agents are described herein.

### A. Small Molecule Therapies

In some embodiments, the present invention provides small molecule inhibitors of galectin (e.g., Gal-3) expression or activity. Exemplary small molecule compounds include, but are not limited to, those disclosed herein (e.g., GM-CT-01 or GR-MD-02; Galectin Therapeutics, Inc, Norcross, Ga.) and derivatives thereof).

In some embodiments, the galectin-3 inhibitor is composed of galactomannan polysaccharide consisting essentially of galactose and mannose residues and resulting from a sufficiently controlled depolymerization of galactomannan so as to result in a homogenous galactomannan polysaccharide (e.g., GM-CT-01). In some embodiments, the galactomannan polysaccharide has an average weight of 4,000 to 60,000 D, as assayed by GPC-MALLS (galactomannan). In some embodiments, the galactomannan polysaccharide composition has a ratio of mannose to galactose molecules in a range of 1:1 to 1:2.5. In some embodiments, the galactomannan polysaccharide composition has a ratio of mannose to galactose molecules of 1.7:1. In some embodiments, the galactomannan polysaccharide composition is produced as described in U.S. Pat. No. 7,893,252, 20120282220A1, and WO 2013101314, each of which is incorporated expressly by reference in its entirety for all purposes. The process is designed to generate a highly pure soluble and homogeneous oligomer with an average molecular weight in the range of about 48,000 Daltons, and mannose to galactose ratio in the range of about 1.7:1. The process incorporates four major phases: controlled depolymerization to produce the desired galactomannan oligomer and three purification steps, removal of insoluble impurities, removal of water soluble impurities, removal of organic soluble impurities, and finally freeze drying to generate a pure and stable form of galactomannan powder. In some embodiments, the product is in the form of a highly soluble oligomer of galactomannan (GM).

Galactomannan can be packaged and delivered as a sterile concentrated solution in a single use vial, while bulk galactomannan can be produced and stored as powder. The process described herein is for both bulk drug and final drug product. The galactomannan drug product can be combined and administered together with a therapeutically effective amount of a therapeutic agent to form the active ingredients of a pharmaceutical preparation. In some embodiments, the drug product can contain normal saline for infusion (about 0.9 M sodium chloride in water) and has a pH of about 6.5.

In some embodiments, the compound is a highly soluble modified polysaccharide so as to be compatible with therapeutic formulations for pluralistic administration via routes including but not limited to intravenous, subcutaneous, intra-articular, inhaled, and oral.

In some embodiments, the galectin-3 inhibitor compound is a polysaccharide that is chemically defined as galacto-rhamnogalacturonate, a selectively depolymerized, branched heteropolymer whose backbone is predominantly comprised of 1,4-linked galacturonic acid (GalA) moieties, with a lesser backbone composition of alternating 1,4-linked GalA and 1,2-linked rhamnose (Rha), which in-turn is linked to any number of side chains, including predominantly 1,4-β-D-galactose (Gal). Other optional side chain minor constituents include arabinose (Ara), xylose (Xyl), glucose (Glu), and fucose (Fuc) (e.g., GR-MD-02). In some embodiments, the compound is a galactose-pronged carbohydrate that is a subtype of galacto-rhamnogalacturonate termed galactoarabino-rhamnogalacturonate, a selectively depolymerized, branched heteropolymer whose backbone is predominantly comprised of 1,4-linked galacturonic acid (GalA) moieties, with a lesser backbone composition of alternating 1,4-linked GalA and 1,2-linked rhamnose (Rha), which in-turn is linked to any number of side chains, including predominantly 1,4-β-D-galactose (Gal) and 1,5apha L arabinose (Ara) residues. Other side chain minor constituents may include xylose (Xyl), glucose (Glu), and fucose (Fuc). In some embodiments, the molar percent of the 1,4-β-D-Gal and 1,5-α-L-Ara residues in the compound of the present invention can exceed 10% of the total molar carbohydrates with approximate ratio ranging from 1:1 to 3:1 respectively. In some embodiments, the molar percent of 1,5-α-L-Ara residues in the compound is zero or only found in trace amounts of up to 1%. In some embodiments, the compound is a polysaccharide chemically defined as galacto-rhamnogalacturonate or galactoarabino-rhamnogalacturonate, a branched heteropolymer with average molecular weight distribution of 2,000 to 80,000, or 20,000 to 70,000, or 5,000 to 55,000 Daltons, as determined by SEC-RI and/or the SEC-MALLS methods. In some embodiments, the compound is a highly soluble modified polysaccharide sufficiently reduced in molecular weight range, for example from about 2,000 to about 80,000 D, so as to be compatible with therapeutic formulations for pluralistic administration via routes including but not limited to intravenous, subcutaneous, intra-articular, inhaled, and oral. In some embodiments the galacto-rhamnogalacturonate compound is produced by the method described in U.S. Pat. No. 8,236,780, 20120282220A1, and WO 2013101314, each of which are incorporated herein by reference in their entirety for all purposes.

In some embodiments, nutraceuticals (e.g., citrus pectin or other pectins) are utilized. In some embodiments, N-acetyllactosamine (N-Lac) (Sigma-Aldrich, St. Louis, Mo.) is utilized.

In some embodiments, the compounds described in Téllez-Sanz, Current Medicinal Chemistry, 2013, 20, 2979-2990; herein incorporated by reference in its entirety. Examples

include but are not limited to:

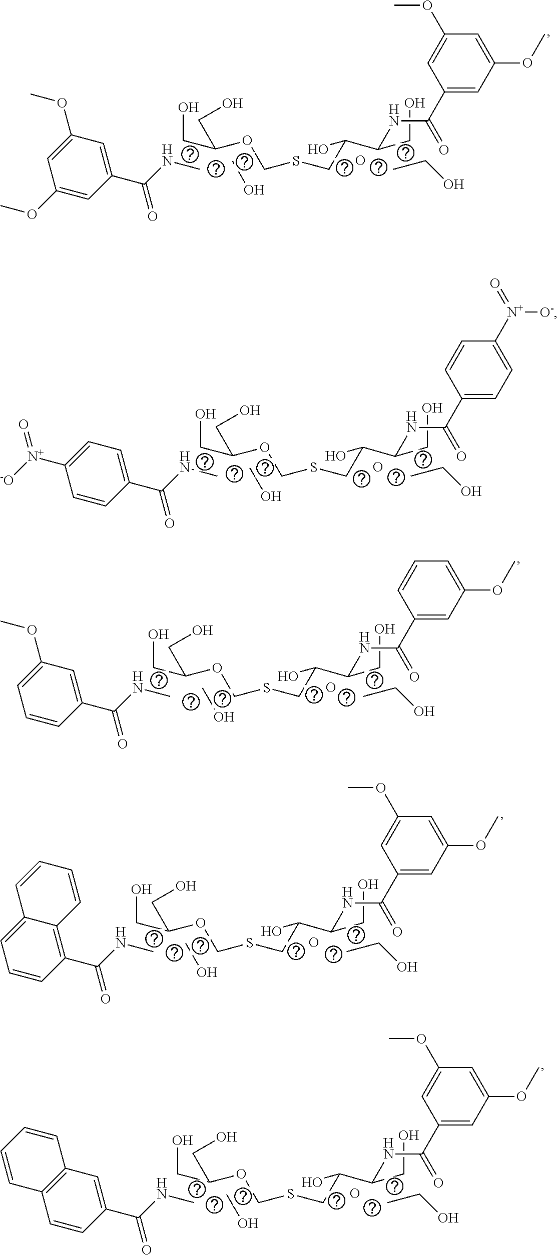

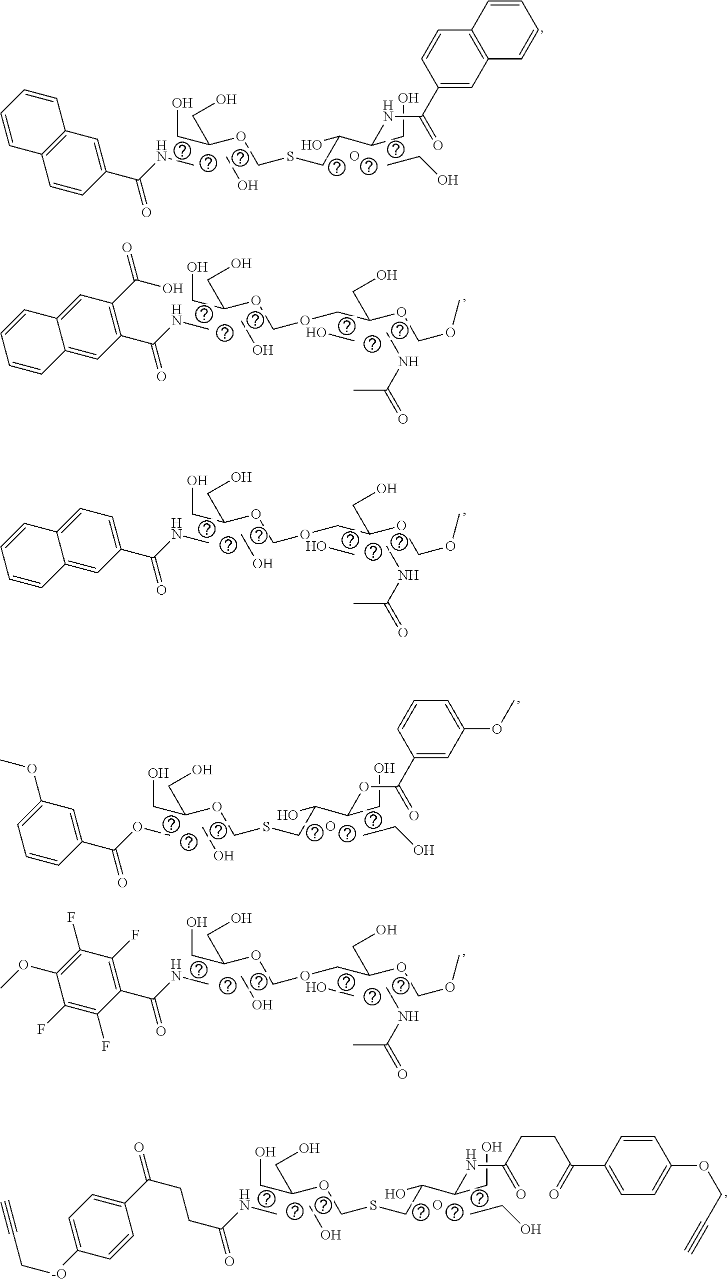

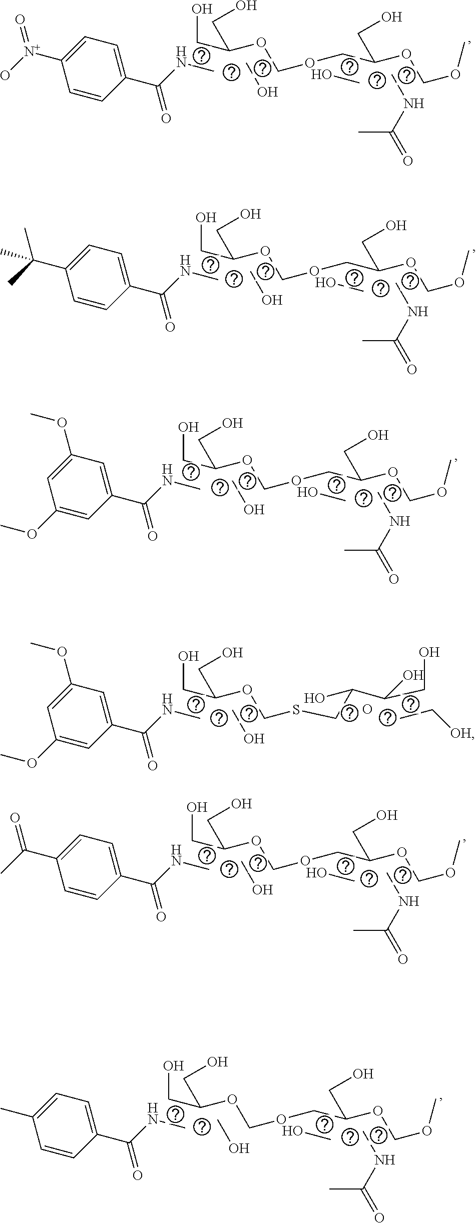

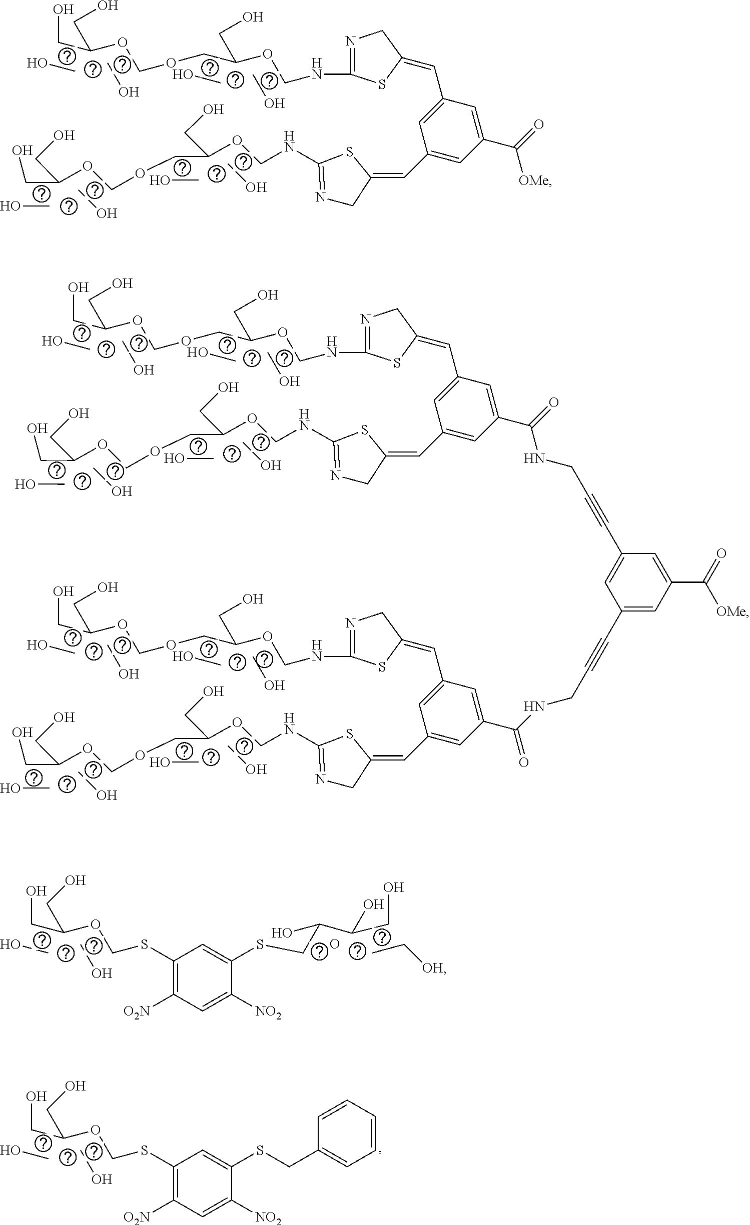

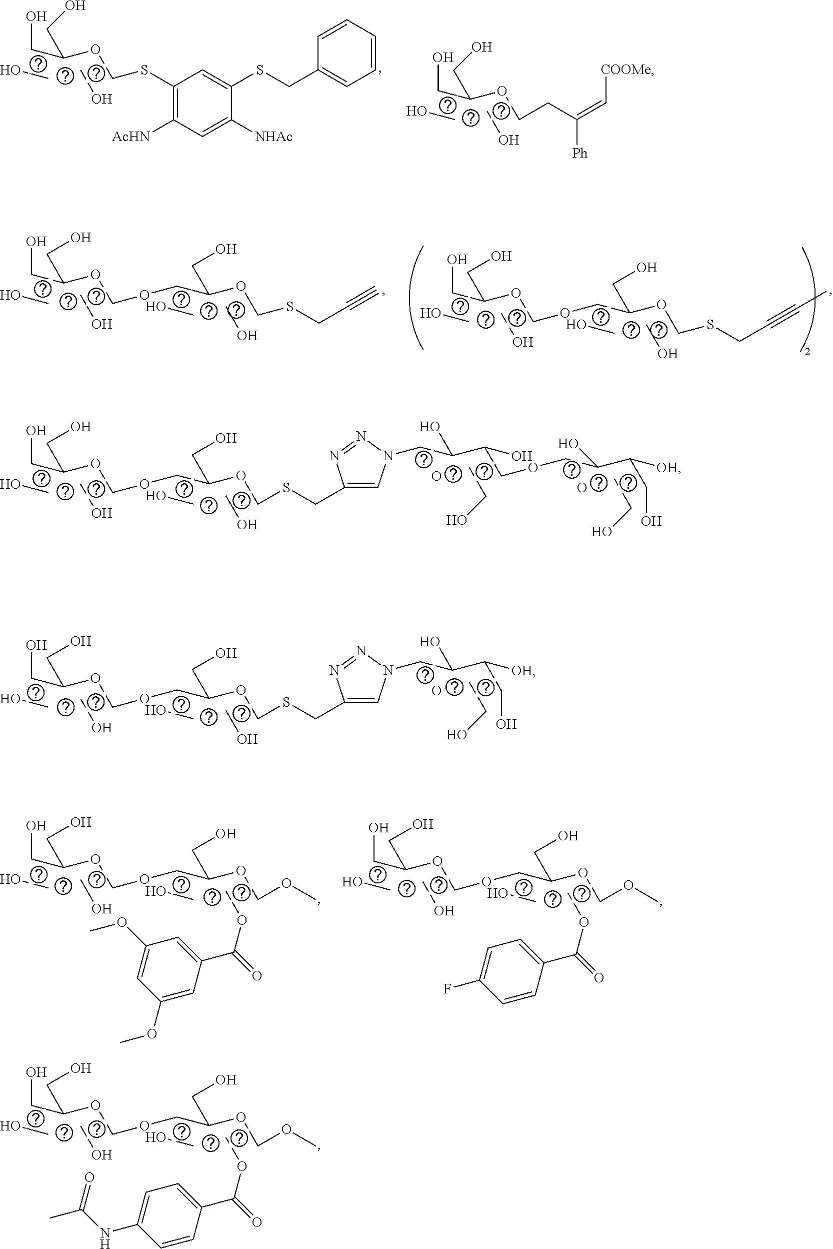

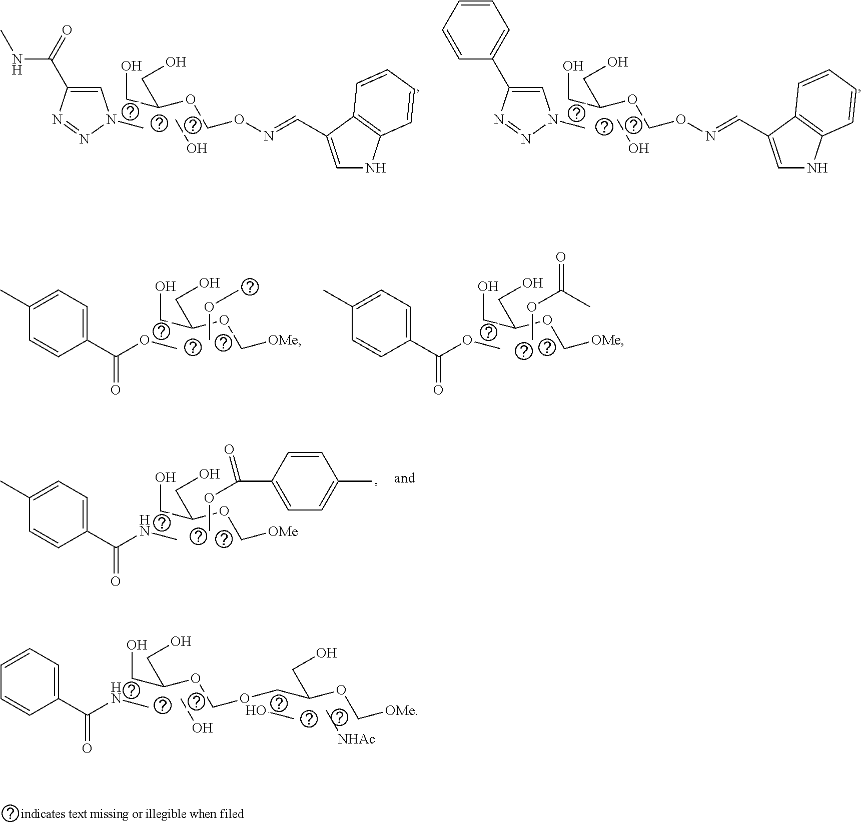

In some embodiments, compounds with the structure

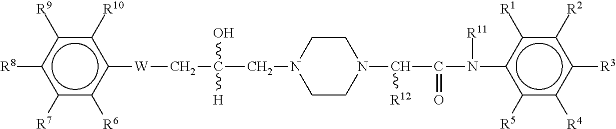

(e.g., Ranolazine; See e.g., U.S. Pat. No. 4,567,264; herein incorporated by reference in its entirety) are utilized. In some embodiments, these compounds find use in the inhibition of galectin (e.g., as a therapeutic for AF), alone or in combination with additional therapeutic agents described herein.

The present invention also provides methods of modifying and derivatizing the compositions of the present invention to increase desirable properties (e.g., binding affinity, activity, solubility and the like), or to minimize undesirable properties (e.g., nonspecific reactivity, toxicity, and the like). The principles of chemical derivatization are well understood. In some embodiments, iterative design and chemical synthesis approaches are used to produce a library of derivatized child compounds from a parent compound. In some embodiments, rational design methods are used to predict and model in silico ligand-receptor interactions prior to confirming results by routine experimentation.

### B. Carbohydrate Therapies

In some embodiments, natural product therapeutics are utilized. In some embodiments, natural products are plant-derived inhibitors of galectin. For example, in some embodiments, inhibitors are pectins, pectic fragments, or derivatives or mimetics thereof. Examples include, but are not limited to, citrus pectin.

### C. RNA Interference and Antisense Therapies

In some embodiments, the present invention targets the expression of galectin. For example, in some embodiments, the present invention employs compositions comprising oligomeric antisense or RNAi compounds, particularly oligonucleotides (e.g., those described herein), for use in modulating the function of nucleic acid molecules encoding galectin, ultimately modulating the amount of galectin expressed.

1. RNA Interference (RNAi)

In some embodiments, siRNA is used to inhibit expression of a galectin (e.g., Gal-3) polypeptide. RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC (RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments. In some embodiments, RNAi oligonucleotides are designed to target the junction region of fusion proteins.

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

An important factor in the design of siRNAs is the presence of accessible sites for siRNA binding. Bahoia et al., (J. Biol. Chem., 2003; 278: 15991-15997; herein incorporated by reference) describe the use of a type of DNA array called a scanning array to find accessible sites in mRNAs for designing effective siRNAs. These arrays comprise oligonucleotides ranging in size from monomers to a certain maximum, usually Comers, synthesized using a physical barrier (mask) by stepwise addition of each base in the sequence. Thus the arrays represent a full oligonucleotide complement of a region of the target gene. Hybridization of the target mRNA to these arrays provides an exhaustive accessibility profile of this region of the target mRNA. Such data are useful in the design of antisense oligonucleotides (ranging from 7 mers to 25 mers), where it is important to achieve a compromise between oligonucleotide length and binding affinity, to retain efficacy and target specificity (Sohail et al, Nucleic Acids Res., 2001; 29(10): 2041-2045). Additional methods and concerns for selecting siRNAs are described for example, in WO 05054270, WO05038054A1, WO03070966A2, J Mol Biol. 2005 May 13; 348(4):883-93, J Mol Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection of siRNAs.

In some embodiments, the present invention utilizes siRNA including blunt ends (See e.g., US20080200420, herein incorporated by reference in its entirety), overhangs (See e.g., US20080269147A1, herein incorporated by reference in its entirety), locked nucleic acids (See e.g., WO2008/006369, WO2008/043753, and WO2008/051306, each of which is herein incorporated by reference in its entirety). In some embodiments, siRNAs are delivered via gene expression or using bacteria (See e.g., Xiang et al., Nature 24: 6 (2006) and WO06066048, each of which is herein incorporated by reference in its entirety).

Chemical modifications can enhance the stability and uptake of naked siRNAs (Choung et al., Biochem Biophys Res Commun. 2006; 342(3):919-927.) siRNAs can be directly modified without impacting their ability to silence their targets. Chemical modifications have been rigorously investigated for virtually every part of siRNA molecules, from the termini and backbone to the sugars and bases, with the goal of engineering siRNA with prolonged half-life and increased cellular uptake. In some embodiments, the sugar moiety is modified. For example, the incorporation of a 2′-fluoro (2′-F), 2′-O-methyl, 2′-halogen, 2′-amine, or 2′-deoxy (Kawasaki et al., J Med Chem. 1993; 36(7):831-841; Rusckowski et al., Antisense Nucleic Acid Drug Dev. 2000; 10(5):333-345; Pieken et al., Science. 1991; 253(5017):314-317; Parrish et al., Mol Cell. 2000; 6(5):1077-1087) can significantly increase the stability of siRNA in serum, as can the bridging of the sugar's 2′- and 4′-positions with a —O—CH2 linker (producing what is called a “locked nucleic acid” or LNA) (Elmen et al., Nucleic Acids Res. 2005; 33(1):439-447). The 2′-F can be introduced through endogenous transcription as opposed to chemical synthesis. In some embodiments, 2′-O-methyl modification of only the sense strand is utilized (Chen et al., RNA. 2008; 14(2):263-274).

2. Antisense

In other embodiments, galectin protein expression is modulated using antisense compounds that specifically hybridize with one or more nucleic acids encoding galectin. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of galectin. In the context of the present invention, “modulation” means either an increase (stimulation) or a decrease (inhibition) in the expression of a gene. For example, expression may be inhibited to prevent AF.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding galectin. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a galectin

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in PCT Publ. No. WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Specific examples of preferred antisense compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts.

In other preferred oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science 254:1497 (1991).

The present invention also includes pharmaceutical compositions and formulations that include the antisense compounds of the present invention as described below.

### D. Genetic Therapy

The present invention contemplates the use of any genetic manipulation for use in modulating the expression of galectin. Examples of genetic manipulation include, but are not limited to, gene knockout (e.g., removing the galectin gene from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct). Genetic therapy may also be used to deliver siRNA or other interfering molecules that are expressed in vivo (e.g., upon stimulation by an inducible promoter (e.g., an androgen-responsive promoter)).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subjects in a variety of ways. For example, in some embodiments of the present invention, vectors are administered into heart muscle using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

### E. Antibody Therapy

In some embodiments, the present invention provides antibodies that target cells (e.g., cardiac cells) that express galectin. Any suitable antibody (e.g., monoclonal, polyclonal, or synthetic) may be utilized in the therapeutic methods disclosed herein. In some embodiments, the antibodies are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, commercially available antibodies against galectin are utilized (e.g., available from Santa Cruz Biotechnology, Inc, Santa Cruz, Calif.).

In some embodiments, the therapeutic antibodies comprise an antibody generated against galectin, wherein the antibody is conjugated to a cytotoxic agent. For certain applications, it is envisioned that the therapeutic agents will be pharmacologic agents that will serve as useful agents for attachment to antibodies, particularly cytotoxic or otherwise anticellular agents having the ability to kill or suppress the growth or cell division of cells. Embodiments of the present invention contemplate the use of any pharmacologic agent that can be conjugated to an antibody, and delivered in active form. Exemplary anticellular agents include chemotherapeutic agents, radioisotopes, and cytotoxins. The therapeutic antibodies may include a variety of cytotoxic moieties, including but not limited to, radioactive isotopes (e.g., iodine-131, iodine-123, technicium-99m, indium-111, rhenium-188, rhenium-186, gallium-67, copper-67, yttrium-90, iodine-125 or astatine-211), hormones such as a steroid, antimetabolites such as cytosines (e.g., arabinoside, fluorouracil, methotrexate or aminopterin; an anthracycline; mitomycin C), vinca alkaloids (e.g., demecolcine; etoposide; mithramycin), and alkylating agent such as chlorambucil or melphalan. Other embodiments include agents such as a coagulant, a cytokine, growth factor, bacterial endotoxin or the lipid A moiety of bacterial endotoxin. For example, in some embodiments, therapeutic agents include plant-, fungus- or bacteria-derived toxin, such as an A chain toxins, a ribosome inactivating protein, α-sarcin, aspergillin, restrictocin, a ribonuclease, diphtheria toxin or pseudomonas exotoxin, to mention just a few examples. In some embodiments, deglycosylated ricin A chain is utilized.

In any event, it is proposed that agents such as these may, if desired, be successfully conjugated to an antibody, in a manner that will allow their targeting, internalization, release or presentation to blood components at the site of the targeted cardiac cells as required using known conjugation technology (See, e.g., Ghose et al., Methods Enzymol., 93:280 [1983]).

For example, in some embodiments the present invention provides immunotoxins targeting galectin. Immunotoxins are conjugates of a specific targeting agent typically a cardiac directed antibody or fragment, with a cytotoxic agent, such as a toxin moiety. The targeting agent directs the toxin to, and thereby selectively kills, cells carrying the targeted antigen. In some embodiments, therapeutic antibodies employ crosslinkers that provide high in vivo stability (Thorpe et al., Cancer Res., 48:6396 [1988]).

In some embodiments, antibody-based therapeutics are formulated as pharmaceutical compositions as described below.

II. Pharmaceutical compositions, formulations, and exemplary administration routes and dosing considerations

Exemplary embodiments of various contemplated medicaments and pharmaceutical or food based compositions are provided below.

Embodiments of the present invention provide methods of using the aforementioned compounds in the inhibition of galectin in cells (e.g., cardiac cells) and in the treatment of AF or prevention of the transition between transient and chronic AF.

A. Preparing Medicaments

The compounds of the present invention are useful in the preparation of medicaments to treat AF. The methods and techniques for preparing medicaments of a compound are well-known in the art. Exemplary pharmaceutical formulations and routes of delivery are described below.

One of skill in the art will appreciate that any one or more of the compounds described herein, including the many specific embodiments, are prepared by applying standard pharmaceutical manufacturing procedures. Such medicaments can be delivered to the subject by using delivery methods that are well-known in the pharmaceutical arts.

B. Exemplary Pharmaceutical Compositions and Formulation

In some embodiments of the present invention, the compositions are administered alone, while in some other embodiments, the compositions are preferably present in a pharmaceutical formulation comprising at least one active ingredient/agent (e.g., galectin inhibitor), as defined above, together with a solid support or alternatively, together with one or more pharmaceutically acceptable carriers and optionally other therapeutic agents. Each carrier should be “acceptable” in the sense that it is compatible with the other ingredients of the formulation and not injurious to the subject.

Contemplated formulations include those suitable oral, rectal, nasal, topical (including transdermal, buccal and sublingual), vaginal, parenteral (including subcutaneous, intramuscular, intravenous and intradermal) and pulmonary administration. In some embodiments, formulations are conveniently presented in unit dosage form and are prepared by any method known in the art of pharmacy. Such methods include the step of bringing into association the active ingredient with the carrier which constitutes one or more accessory ingredients. In general, the formulations are prepared by uniformly and intimately bringing into association (e.g., mixing) the active ingredient with liquid carriers or finely divided solid carriers or both, and then if necessary shaping the product.

Formulations of the present invention suitable for oral administration may be presented as discrete units such as capsules, cachets or tablets, wherein each preferably contains a predetermined amount of the active ingredient; as a powder or granules; as a solution or suspension in an aqueous or non-aqueous liquid; or as an oil-in-water liquid emulsion or a water-in-oil liquid emulsion. In some embodiments, the active ingredient is presented as a bolus, electuary, or paste, etc.

In some embodiments, tablets comprise at least one active ingredient and optionally one or more accessory agents/carriers are made by compressing or molding the respective agents. In some embodiments, compressed tablets are prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets are made by molding in a suitable machine a mixture of the powdered compound (e.g., active ingredient) moistened with an inert liquid diluent. Tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention are optionally formulated as ointments, creams, suspensions, lotions, powders, solutions, pastes, gels, sprays, aerosols or oils. In alternatively embodiments, topical formulations comprise patches or dressings such as a bandage or adhesive plasters impregnated with active ingredient(s), and optionally one or more excipients or diluents. In some embodiments, the topical formulations include a compound(s) that enhances absorption or penetration of the active agent(s) through the skin or other affected areas. Examples of such dermal penetration enhancers include dimethylsulfoxide (DMSO) and related analogues.

If desired, the aqueous phase of a cream base includes, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof.

In some embodiments, oily phase emulsions of this invention are constituted from known ingredients in an known manner. This phase typically comprises an lone emulsifier (otherwise known as an emulgent), it is also desirable in some embodiments for this phase to further comprises a mixture of at least one emulsifier with a fat or an oil or with both a fat and an oil.

Preferably, a hydrophilic emulsifier is included together with a lipophilic emulsifier so as to act as a stabilizer. It some embodiments it is also preferable to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulfate.

The choice of suitable oils or fats for the formulation is based on achieving the desired properties (e.g., cosmetic properties), since the solubility of the active compound/agent in most oils likely to be used in pharmaceutical emulsion formulations is very low. Thus creams should preferably be a non-greasy, non-staining and washable products with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with suitable base comprising, for example, cocoa butter or a salicylate.

Formulations suitable for vaginal administration may be presented as pessaries, creams, gels, pastes, foams or spray formulations containing in addition to the agent, such carriers as are known in the art to be appropriate.

Formulations suitable for nasal administration, wherein the carrier is a solid, include coarse powders having a particle size, for example, in the range of about 20 to about 500 microns which are administered in the manner in which snuff is taken, i.e., by rapid inhalation (e.g., forced) through the nasal passage from a container of the powder held close up to the nose. Other suitable formulations wherein the carrier is a liquid for administration include, but are not limited to, nasal sprays, drops, or aerosols by nebulizer, an include aqueous or oily solutions of the agents.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. In some embodiments, the formulations are presented/formulated in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily subdose, as herein above-recited, or an appropriate fraction thereof, of an agent.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies. Still other formulations optionally include food additives (suitable sweeteners, flavorings, colorings, etc.), phytonutrients (e.g., flax seed oil), minerals (e.g., Ca, Fe, K, etc.), vitamins, and other acceptable compositions (e.g., conjugated linoelic acid), extenders, and stabilizers, etc.

C. Exemplary Administration Routes and Dosing Considerations

Various delivery systems are known and can be used to administer a therapeutic agent (e.g., galectin inhibitor), e.g., encapsulation in liposomes, microparticles, microcapsules, receptor-mediated endocytosis, and the like. Methods of delivery include, but are not limited to, intra-arterial, intra-muscular, intravenous, intranasal, and oral routes. In specific embodiments, it may be desirable to administer the pharmaceutical compositions of the invention locally to the area in need of treatment; this may be achieved by, for example, and not by way of limitation, local infusion during surgery, injection, or by means of a catheter.

The agents identified herein as effective for their intended purpose can be administered to subjects or individuals diagnosed with AF. When the agent is administered to a subject such as a mouse, a rat or a human patient, the agent can be added to a pharmaceutically acceptable carrier and systemically or topically administered to the subject.

In some embodiments, in vivo administration is effected in one dose, continuously or intermittently throughout the course of treatment. Methods of determining the most effective means and dosage of administration are well known to those of skill in the art and vary with the composition used for therapy, the purpose of the therapy, the target cell being treated, and the subject being treated. Single or multiple administrations are carried out with the dose level and pattern being selected by the treating physician.

Suitable dosage formulations and methods of administering the agents are readily determined by those of skill in the art. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to an agent of the present invention, the pharmaceutical compositions can also contain other pharmaceutically active compounds or a plurality of compounds of the invention.

More particularly, an agent of the present invention also referred to herein as the active ingredient, may be administered for therapy by any suitable route including, but not limited to, oral, rectal, nasal, topical (including, but not limited to, transdermal, aerosol, buccal and sublingual), vaginal, parental (including, but not limited to, subcutaneous, intramuscular, intravenous and intradermal) and pulmonary. It is also appreciated that the preferred route varies with the condition and age of the recipient, and the disease being treated.

In some embodiments, agents are administered intravenously. In some embodiments, agents are formulated in Cremophor (BASF, Parsippany, N.J.)

Ideally, the agent should be administered to achieve peak concentrations of the active compound at sites of disease. This may be achieved, for example, by the intravenous injection of the agent, optionally in saline, or orally administered, for example, as a tablet, capsule or syrup containing the active ingredient.

Desirable blood levels of the agent may be maintained by a continuous infusion to provide a therapeutic amount of the active ingredient within disease tissue. The use of operative combinations is contemplated to provide therapeutic combinations requiring a lower total dosage of each component antiviral agent than may be required when each individual therapeutic compound or drug is used alone, thereby reducing adverse effects.

D. Exemplary Co-Administration Routes and Dosing Considerations

The present invention also includes methods involving co-administration of the compounds described herein with one or more additional active agents (e.g., agents useful in the treatment of AF). Indeed, it is a further aspect of this invention to provide methods for enhancing prior art therapies and/or pharmaceutical compositions by co-administering a compound of this invention. In co-administration procedures, the agents may be administered concurrently or sequentially. In one embodiment, the compounds described herein are administered prior to the other active agent(s). The pharmaceutical formulations and modes of administration may be any of those described above. In addition, the two or more co-administered chemical agents, biological agents or other treatments may each be administered using different modes or different formulations.

E. Exemplary Use as Food Additives or Supplement

In some embodiments of the present invention, galectin inhibitors (e.g., natural products derived from plant based materials) are administered in the form of supplements or food additive. Exemplary delivery forms include, but are not limited to, tablet, capsule, powder, drops or syrup. In some embodiments, galectin inhibitors (e.g., pectins or pectin derivatives) are included in vitamins or other nutritional supplements containing additional active ingredients (e.g., vitamins or supplements).

In some embodiments, therapeutics are added to food or food products (e.g., prepared foods or food products). Examples of food products include, but are not limited to, diet drinks, diet bars, supplements, prepared frozen meals, candy, snack products (e.g., chips), prepared meat products, milk, cheese, yogurt and the like.

In some embodiments, increased concentrations of natural galectin inhibitors in a food product are achieved through methods of selecting edible plant-based ingredients rich in inhibitors and processing these ingredients in a manner to enhance its inhibitor effects. In some embodiments, concentrated plant-derived inhibitors are added to food products to create functional foods. In some embodiments, specific nutritional recommendations as part of a food or nutrition program are used to reduce the risk of atrial fibrillation or enhance its treatment (e.g., by consuming named plant-based products rich in such inhibitors).

### III. Therapeutic Methods

As described herein, embodiments of the present invention find use in treating atrial fibrillation or preventing permanent or persistent AF (e.g., by preventing the transition from paroxysmal to persistent AF) by blocking at least one biological activity of galectin (e.g., Gal-3 or Gal-2). Exemplary dosing and administration schedules are described herein.

In some embodiments, galectin targeting agents are administered to a subject diagnosed with or at risk of atrial fibrillation. In some embodiments, diagnostic assays for AF are performed prior to administering the agents. In some embodiments, agents are administered as continuous or maintenance therapies to prevent initial or recurrent AF events.

In some embodiments, subjects with AF or at risk of AF are treated with a galectin blocking agent and are then tested for signs or symptoms of AF. In some embodiments, the results of the test is used to adjust (e.g., increase, decrease, start or stop) treatment with the galectin blocking agent.

In some embodiments, subjects with AF or at risk of AF are tested for signs and/or symptoms of AF. In some embodiments, the results of the test is used to adjust (e.g., start or stop) treatment with the galectin blocking agent. In some embodiments, subjects are then tested again and a treatment course of action is determined (e.g., increase, decrease, start or stop treatment).

In some embodiments, subjects have been diagnosed with paroxysmal or early AF. In some embodiments, administration of an agent that targets galectin prevents progression to persistent or permanent AF. In some embodiments, administration of the agent prevents the development of fibrosis associated with AF.

In some embodiments, the subject does not exhibit signs or symptoms and/or is not currently undergoing treatment of fibrosis (e.g., liver fibrosis) or cancer (e.g., colorectal cancer or melanoma).

### IV. Drug Screens

In some embodiments of the present invention, the compounds of the present invention, and other potentially useful compounds, are screened for their biological activity (e.g., ability to block galectin or treat and/or prevent AF or progression of AF from early to permanent or persistent AF).

In some embodiments, structure-based virtual screening methodologies are contemplated for identifying galectin inhibitors. For example, in some embodiments, molecular modeling is used to identify inhibitors. In some embodiments, modeling is used to identify compounds that inhibit the activity of galectin or galectin pathway components.

In some embodiments, compounds are screened in cell culture or in vivo (e.g., non-human or human mammals) for their ability to inhibit galectin. In some embodiments, screens detecting expression or inhibition of expression of downstream signaling molecules.

In some embodiments, the present invention provides high throughput screening of test compounds. For example, in some embodiments, large numbers of different test compounds (e.g., from a test compound library) are provided (e.g. attached to or synthesized) on a solid substrate. Test compounds can be reacted with cardiac cells, or portions thereof, and washed. Bound cells are then detected by methods well known in the art, using commercially available machinery and methods (e.g., the Automated Assay Optimization (AAO) software platforms (Beckman, USA) that interface with liquid handlers to enable direct statistical analysis that optimizes the assays; modular systems from CRS Robotics Corp. Burlington, Ontario), liquid handling systems, readers, and incubators, from various companies using POLARA (CRS), an open architecture laboratory automation software for a Ultra High Throughput Screening System; 3P (Plug&Play Peripherals) technology, which is designed to allow the user to reconfigure the automation platform by plugging in new instruments (ROBOCON, Vienna, Austria); the Allegro system or STACCATO workstation (Zymark), which enables a wide range of discovery applications, including HTS, ultra HTS, and high-speed plate preparation; MICROLAB Vector software (Hamilton Co., Reno, Nev., USA) for laboratory automation programming and integration; and others).

In some embodiments, assays measure a response the target cells (cardiac cells) provide (e.g., detectable evidence that a test compound may be efficacious). In some embodiments, the detectable signal is compared to control cells and the detectable signal identified by subtraction analysis. The relative abundance of the differences between the “targeted” and “untargeted” samples can be compared.

In some embodiments, the present disclosure provides a method of screening compounds in a non-human animal (e.g., the ovine model of AF described herein). For example, in some embodiments, test compounds are administered to a non-human animal (e.g., ovine) that exhibits a transition from paroxysmal to long-standing persistent atrial fibrillation; and b) identifying compounds that inhibit or delay the transition. In some embodiments, the paroxysmal and self-sustained AF is induced by atrial tachypacing (e.g., burst tachypacing). In some embodiments, the test compound is administered prior to a first episode of atrial fibrillation. In some embodiments, the test compound is administered repeatedly.

### EXAMPLES

The following examples are provided to demonstrate and further illustrate certain embodiments of the present invention and are not to be construed as limiting the scope thereof

### Example 1

### TGF-β1 and Gal-3 are Expressed in Adult Sheep Atrial Fibroblasts

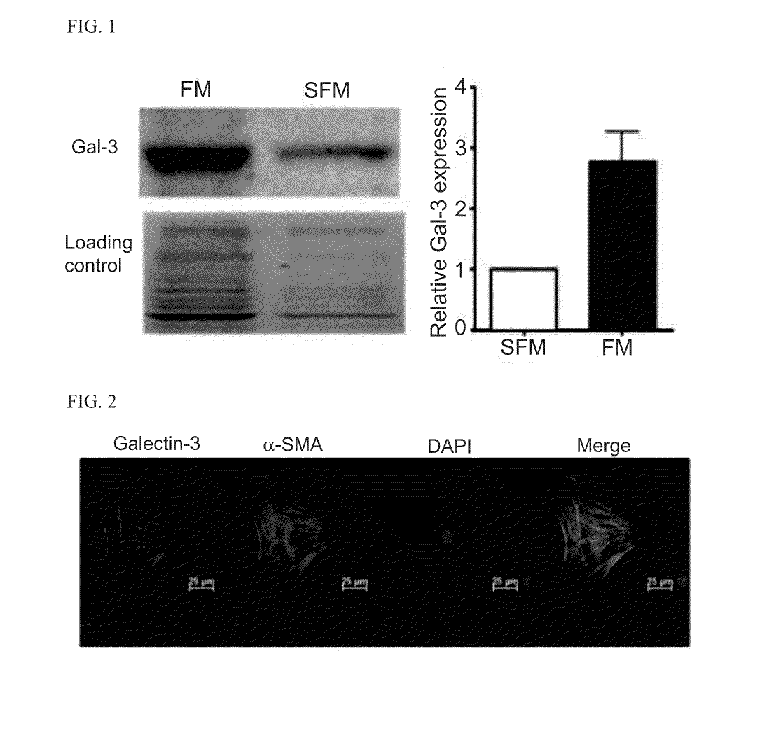

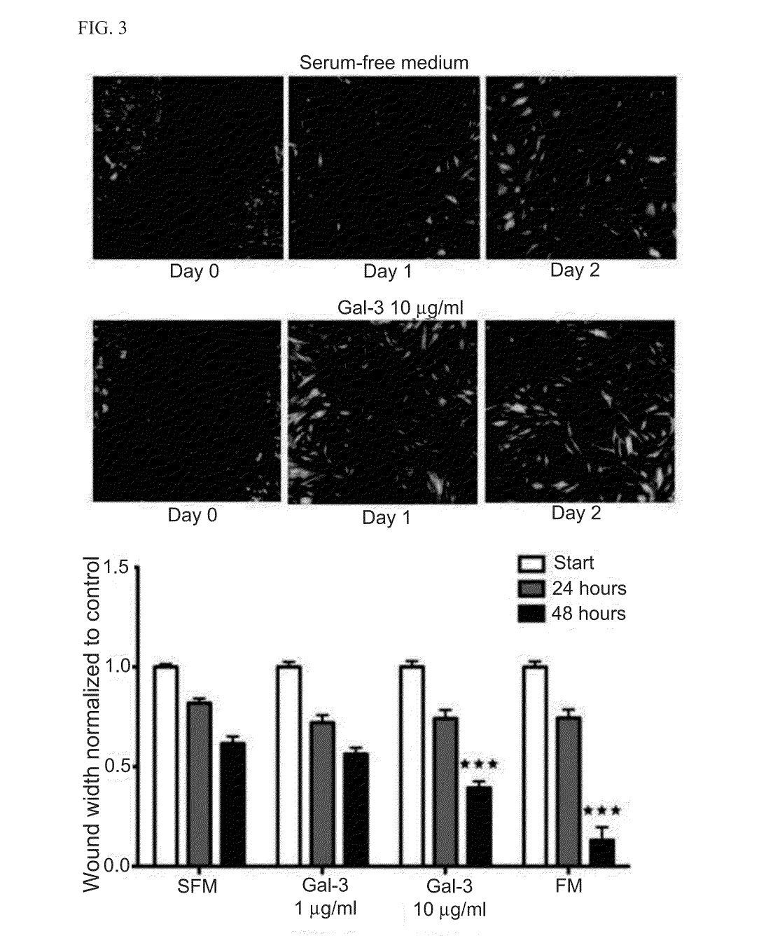

The concentration of TGF-01 in was measured in FBS-free supernatant of cultured fibroblasts harvested from the atria of adult sheep by TGF-β1 ELISA kit (R&D systems, MN, USA). Fibroblasts were obtained and cultured as described elsewhere (Vaidyanathan et al., J Biol Chem. 2010; 285:28000-28009). Next, the expression of LGALS3, the gene coding Gal-3, isolated from purified myofibroblasts from the left atrial appendage (LAA) grown in full medium (FM) and serum free medium (SFM) was measured. PCR results showed that LAA myofibroblasts expressed LGALS3. The expression of LGALS3 was further confirmed by sequencing the product. The sequence was a 100% match to the uncharacterized ovine sequence, thus confirming that sheep cardiac myofibroblasts express the gene coding Gal-3. In addition, myofibroblasts grown in FM (0.62±0.13) and SFM (0.42±0.1) expressed similar levels of LGALS3 mRNA normalized to GAPDH expression. Furthermore, the expression of Gal-3 protein was shown by western blot analysis in cardiac myofibroblast (FIG. 1, left). Quantification showed that FM increased Gal-3 protein expression (FIG. 1, right), which is due to an increased cell density in FM cultures. Immunohistochemistry was then used to further confirm the presence of Gal-3 in adult sheep atrial myofibroblast. As shown in FIG. 2, sheep atrial myofibroblasts were characterized by their high levels of expression of α-SMA at stress fibers.

### Gal-3 Increases In Vitro Migration and Proliferation of Atrial Myofibroblasts.

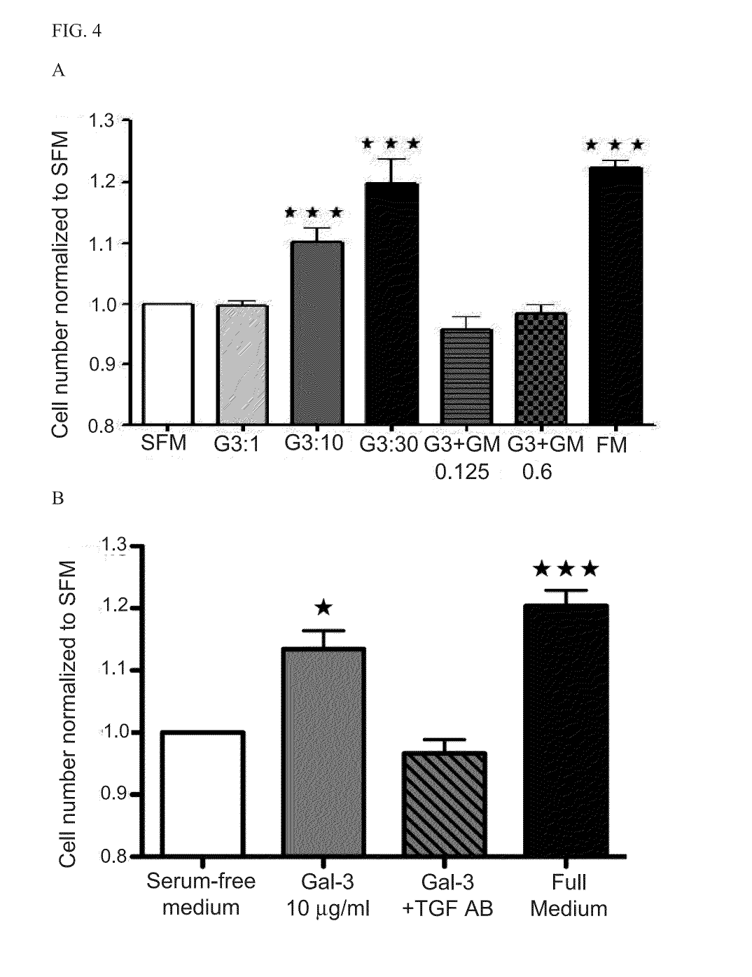

Wound healing experiments were performed to investigate if exogenous Gal-3 can increase myofibroblast migration in vitro (FIG. 3). Adult sheep atrial fibroblasts were cultured in 96-well plates until confluent. Before serum starvation, cells were stained with cell tracker dye and DAPI. A wound was created by scraping a semi-confluent monolayer of cells with a 1 ml pipette tip. Thereafter, cells underwent serum starvation in DMEM with 5% FBS for 6 hrs. Comparisons were made between untreated and Gal-3 treated (1 μg/ml or 10 μg ml) cells. The wound was imaged at 4 different locations at 3 different time points (days 0, 1 and 2). The size of the wound was quantified using the NIS element program by Nikon. As demonstrated in FIG. 3, 10 μg/ml Gal-3 significantly increased the rate of migration and promoted wound healing. Next, to investigate the effects of Gal-3 on myofibroblast proliferation, adult sheep atrial fibroblasts were treated with recombinant human Gal-3 (EMD chemicals, USA) for 24 hours in the presence of SFM. FM was included as a positive control. Cell proliferation was measured using cell proliferation reagent WST (Roche Diagnostics, USA). As shown in FIG. 4, Gal-3 (1-30 μg/ml) increased the number of adult sheep atrial myofibroblasts in a concentration-dependent manner. Such concentrations are in the same range used previously (Sharma et al., Circulation. 2004; 110:3121-3128) to investigate Gal-3 induced fibroblast proliferation in the cardiomyopathic heart. In the presence of two different concentrations (0.125 and 0.6 mg/ml) of Gla-3 inhibitor (GM-CT-01), Gal-3 failed to increase sheep atrial myofibroblast proliferation (FIG. 4).

Altogether, these results provide evidence for involvement of Gal-3 in the pathophysiology of atrial fibrosis. In addition, the results strongly support the therapeutic value of Gal-3 inhibitors.

### Gal-3 Inhibition Prevents PAF-Induced Structural Remodeling in the Atria of PAF Sheep.

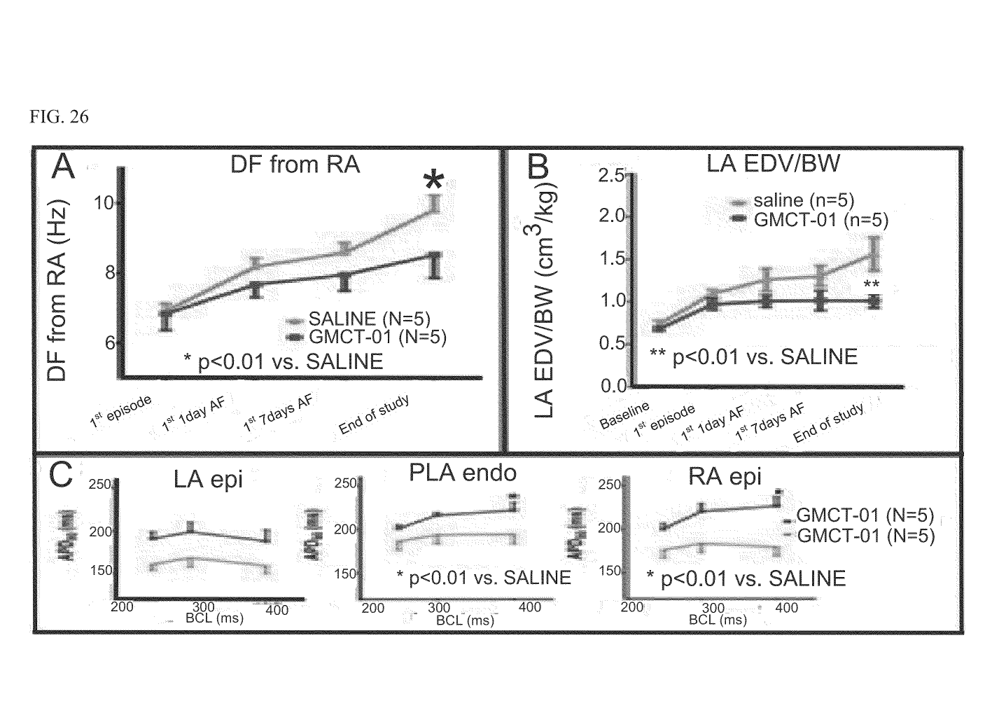

Gal-3 inhibition shows beneficial anti-fibrotic effects in various organ systems including the heart (de Boer et al., Curr Heart Fail Rep. 2010; 7:1-8). To determine the role of TGF-β1 and Gal-3 in electrical remodeling, a blinded study was conducted in in 10 sheep in which the Gal-3 inhibitor GMCT-01 (12 mg/Kg) was administered twice per week. The time course of dominant frequency (DF) (Filgueiras-Rama et al., Circ Arrhythm Electrophysiol. 2012; 5:1160-1167) changes and atrial area in the presence (N=5) and the absence (N=5) of the drug were measured. As illustrated in FIGS. 26A and B, as AF progressed from paroxysmal to persistent, GMCT-01 significantly reduced the rate of DF increase and the maximum DF, as well as the rate of dilatation of the left atrium adjusted to body weight. Optical mapping of the atria at the end of the study showed that GMCT-01 prolonged the action potential duration (APD90) (FIG. 26C). In addition, the shortest cycle length for 1:1 conduction during constant right atrial pacing was also prolonged in the presence of GMCT-01 (206±11 ms vs. 164±9 ms, p=0.02).

### Example 2

### Ovine Model of Long-Term PAF

A single-chamber pacemaker canister (St Jude Medical, Mn, USA) with a lead inserted into the right atrium (RA) and a subcutaneous loop recorder (IRL; Reveal® XT, Medtronic) was implanted in sheep. This created a sheep model of long-term PAF. The median time to 1st AF episode is 13 days, paroxysmal AF is 7 weeks and persistent AF is 9 weeks. Thereafter, PAF is allowed to continue for a follow up (FU) period ≧6 months. During this time, PAF is self-sustained. The DF of the first AF episode was 7.7±0.7 Hz. It significantly increased during the time of paroxysmal AF until persistent AF was established (10.1±1.2 Hz, p<0.001). No additional significant increase in DF was noted after 30 weeks of self-sustained persistent AF (10.7±0.7 Hz). By using these PAF sheep, fibrillatory DF increase and the relationship between the development of fibrosis, the increased fibrillatory DF and the changes in the expression of Gal-3/TGF-β1 during the transition from paroxysmal AF to PAF are investigated.

### Gal-3/TGF-β1 Expression and PAF Induced Structural Remodeling.

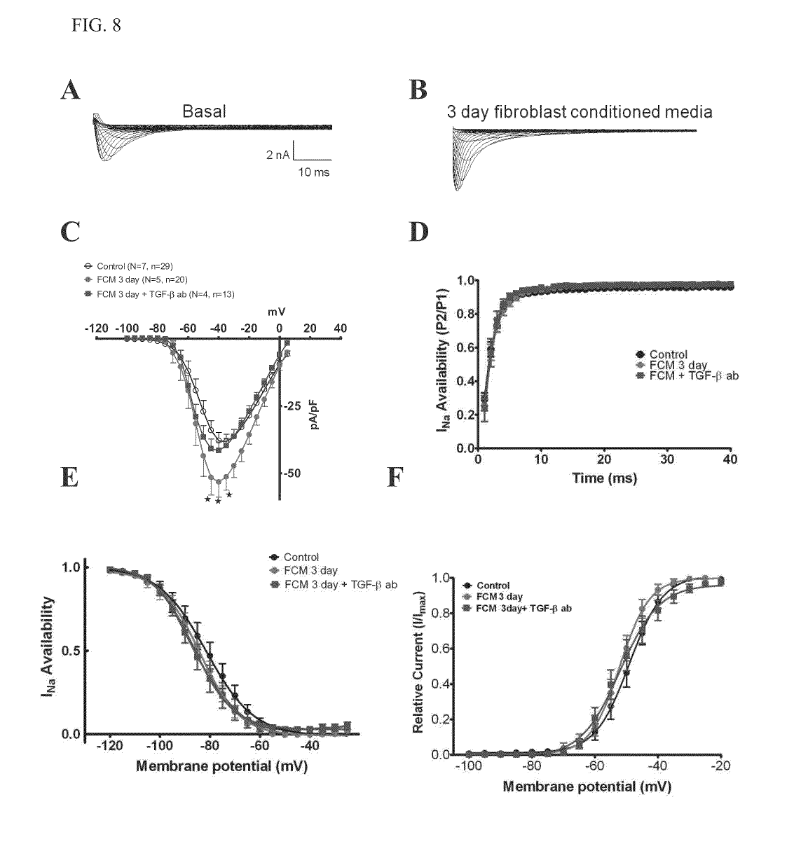

The expression of α-smooth muscle actin (α-SMA) and degree of interstitial fibrosis in three major regions of the RA and LA of PAF sheep was evaluated. Both of them were significantly increased in PAF sheep compared to control sheep (FIG. 7). Next, the concentration of TGF-β1 and Gal-3 in LAA harvested from control and PAF hearts was measured using human TGF-β1 and Gal-3 ELISA kits in order to investigate whether the fibrosis and increased α-SMA observed in the PAF are accompanied by increases in TGF-β1 and Gal-3. As shown in FIG. 8 the respective tissue concentrations of TGF-β1 and Gal-3 in LAA were, respectively, ˜twice and ˜2.5 times larger than control. Moreover, during the transition to PAF there is a progressive increase in amino peptide of procollagen type III (PIIINP), a well-known marker of collagen turnover and cardiac fibrosis (Martos et al., Eur J Heart Fail. 2009; 11:191-197). PIIINP levels are significantly increased (˜twice) in AF sheep compared with Sham.

Altogether, the results presented above demonstrate that this model of chronic tachypacing leading to long-term self-sustained PAF in the sheep is clinically relevant.

### Example 3

### Gal-3 is an Essential Mediator of TGF-β1 Induced-Atrial Structural Remodeling

Myofibroblasts are active contributors to fibrosis (Souders et al., Circ Res. 2009; 105:1164-1176), which is part of the maladaptive atrial response to AF (He et al., Circ Res. 2011; 108:164-175). Gal-3 expression has been shown to be temporarily and spatially associated with fibrosis (Henderson et al., Proc Natl Acad Sci USA. 2006; 103:5060-5065), being minimal in normal liver, maximal at peak fibrosis, and virtually absent after recovery from fibrosis. Gal-3 is a pleiotropic molecule found in the nucleus, cytoplasm, and at the cell surface, where Gal-3 pentamers bind to poly N-acetyl lactosamine (LNac) residues on TGF-β receptors of fibroblasts causing cell surface retention and promoting its signaling through Smads and Akt (Mackinnon et al., Am J Respir Crit Care Med. 2011; Bonniaud et al., J Immunol. 2005; 175:5390-5395). The effects, signaling pathways and transcription factors involved in the Gal-3 regulation of TGF-β1 driven sheep atrial myofibroblast activation, migration, and collagen production are investigated in vitro. The present invention is not limited to a particular mechanism. Indeed, an understanding of the mechanism is not necessary to practice the present invention. Nonetheless, it is contemplated that Gal-3 regulates the effects of TGF-β1 by promoting the retention of TGFβR-II on the surface membrane of the atrial myofibroblast, thus acting to increase transcription of pro-fibrotic molecules via stimulation of phosphorylation and nuclear translocation of the Smad2/3 complex (FIG. 7). Cell culture, quantitative real-time-PCR, western blotting, immunohistochemistry, confocal microscopy, small interference RNA transfection, wound healing assay, and TGFβR-II flow cytometry is used to confirm.

### Role of Gal-3 in Retention of TGFβR-II on the Surface of Atrial Myocytes and Myofibroblasts.

It is first investigated if exogenous Gal-3 treatment increases the TGFβR-II levels on cell surface. This is attained by using antibodies specific to the extracellular domain of the receptor in flowcytometer and using Immunolocalization on confocal microscope. These assays are done in both detergent treated and untreated cells to determine both extracellular and total receptor expression in these cells. The cells are also treated with Gal-3 inhibitors to understand the role of endogenous Gal-3 on TGF receptor cell surface expression.

### Gal-3 Regulation of the Effects of TGF-β1 in Atrial Myofibroblasts Via Increased Phosphorylation and Nuclear Translocation of the Smad2/3 Complex.

To investigate whether Gal-3 treatment can increase TGF-β1-induced Smad2/Smad3 phosphorylation in sheep atrial myofibroblasts and myocytes, cells are treated with TGF-β1 in presence and absence of Galectin for 30 minutes. Phosphorylation and nuclear translocation of the complex are assessed by Western blot and Immunolocalization (using phosphospecific antibody to Smad2 and Smad3). Small interference RNAs mediated knockdown of human Gal-3 is used to investigate the role of endogenous Gal-3 in TGF-β1-induced Smad2/Smad3 phosphorylation in sheep atrial myofibroblasts and myocytes. It is also verified whether or not the effects of Gal-3 in sheep atrial myofibroblasts are mediated through downregulation of Smad7, which inhibits TGF-β1 signaling as was shown for rabbit (He et al., Circ Res. 2011; 108:164-175; Zhao et al., Mech Dev. 2000; 93:688-697). Cell lysates are immunoblotted Smad (de Boer et al., supra). Western blots will be normalized against GAPDH.

### In Vitro Study.

RT-PCR: RNA is extracted using a Qiagen RNeasy minikit, and transcribed using an Invitrogen Superscript III first strand synthesis kit. Human or bovine primers is selected to cross an intron within the DNA sequence of interest. Twenty-five μl reactions using SyberGreenE® (Invitrogen) will be run in duplicate on a BioRad iCycler 96 well plate. The cycles to threshold (cT) values corresponding to mRNA levels are based on a log scale, and will be transformed to delta cT values by subtracting the gene of interest from 18 s rRNA, which is present in all cells, and accounts for variability in RNA quality (Swartz et al., Heart Rhythm. 2009; 6:1415-1422).

SDS-PAGE and Immunoblotting: SDS-PAGE is carried out as described (Laemmli, Nature 1970; 227:680-5). Briefly, samples are run on 4-12% acrylamide gels (Invitrogen), transferred to nitrocellulose (Bio-Rad) in a Hoeffer transfer apparatus immersed in Tris-glycine buffer (Fisher BioReagents); 0.005% SDS is added to the transfer buffer when blots are run for detection of Gal-3. Nonspecific binding sites are incubated in blocking buffer (5% nonfat dry milk (NFM) in PBS with Tween-20 (0.05%)). Membranes are then incubated overnight with specific primary antibodies at 4° C. After washing, membranes will be incubated with peroxidase-conjugated secondary antibodies. Antigen complexes are visualized using enhanced chemiluminescence (Pierce) and autoradiography. GAPDH is used as a loading control. Protein bands are quantified by digital densitometry with a BioRad Fluor-S imager and Quantity One software (Bio-Rad). At least three Western blots are used for quantification for each experiment. Immunohistochemistry: Atrial sheep myofibroblasts are plated on coverslips. After treatment cells are fixed with 3% paraformaldehyde. Cells are blocked with 10% NGS and consequently treated with primary antibodies followed by secondary antibodies. Thereafter cells are stained for DAPI and the coverslips are fixed with mounting media. Images are taken using confocal imaging with sequential laser firing using an Olympus FluoView confocal laser scanning microscope (A1R/A1, Nikon, Tokyo, Japan).

Small interference RNA transfection: Small interference RNAs (siRNAs) specific for Galectin-3 are purchased from Dharmacon or other suitable supplier (Chicago, USA). Cardiac myocytes and fibroblasts are transfected with ON-TARGET plus SMART pool siRNA for 48 hours pretreatment with TGF-β1. Control for transfection is provided by ON-TARGET plus Non-targeting siRNA. All transfections are performed as per manufacturer's protocol, using Dharmafect 1.

Wound Healing Assay: Described Above (FIG. 3)

TGF receptor flow cytometry: For determination of cell surface expression TGFβRII, Anti-mouse TGF-β RII capable of recognizing the extracellular domain (R and D systems, MN, USA) of the receptor is be used. Briefly treated and fixed cells are blocked with 1 μg of mouse IgG for 15 minutes at room temperature. After blocking, cells are incubated with conjugated antibody for 30 minutes at room temperature. Unbound antibody is removed by washing the cells twice in Flow Cytometry Staining Buffer. The cells are resuspended in Flow Cytometry Staining Buffer for final flow cytometric analysis. Cells treated with APC-labeled goat IgG antibody are used as control for this analysis.

Single cell RT-PCR: Single cells are harvested using coated glass micropipettes under microscope and put into a 0.2 ml tube containing 5 μl DNase-free and RNase-free distilled water and 10 units of RNase inhibitor (Applied Biosystems, USA). Reverse transcription is performed using the SuperScript III First-Strand System for RT-PCR kit (Invitrogen, USA). Real time PCR will be carried out in a final volume of 50 μl consisting of 10 μl of cDNA, TaqMan Universal PCR Master Mix (Applied Biosystems). mRNA relative expression levels are determined using the ΔΔCt method.

### Example 4

### Involvement of Gal-3 in the Pathophysiology of PAF in a Clinically Relevant Sheep Heart Model

Recently, it has been shown that long-term rapid atrial pacing in rabbits induces myocardial fibrosis through the Ang II type 1 receptor-coupled TGF-β1/Smad signaling pathway (He et al., Circ Res. 2011; 108:164-175). Atrial tachypacing for ˜9 weeks resulted in PAF for over six months, which was accompanied by progressive acceleration of AF dominant frequency and by increased expression of Gal-3, TGF-β1 and α-smooth muscle actin (α-SMA) proteins (see above). Gal-3 and TGF-β1 share signaling pathways with Ang II to stimulate fibroblast activity. Experiments are conducted to demonstrate that Gal-3 is a regulator of the TGF-β1 induced atrial fibrosis that contributes to the perpetuation of PAF by anchoring AF sources at specific atrial locations. Daily IV doses of Gal-3 inhibitor are administered during atrial tachypacing.

Relationship Between Serum Gal-3 and Other Serum Markers of Atrial Fibrosis in Sheep with PAF.
Gal-3 has been significantly correlated with serum markers of cardiac collagen turnover and fibrosis in heart failure patients. The atria of PAF sheep show significant increases in interstitial fibrosis, as well as TGF-β1 and Gal-3. Therefore, the impact of Gal-3 and TGF-β1 on serum markers of atrial fibrosis is assessed.

### Correlation Between Gal-3/TGF-β1 Induced Fibrosis and PAF Perpetuation as Demonstrated by Anchoring AF Sources at Specific Atrial Locations.

Immunohistochemical experiments are conducted to measure tissue expression of α-SMA as a marker of myofibroblast proliferation as well as TGF-β1 and Gal-3 in the atria of control and PAF sheep. Histological analyses with picrosirius red are conducted as previously described (Tanaka et al., Circ Res. 2007; 101:839-847; Berenfeld et al., Heart Rhythm. 2011; 8:1758-1765) to quantify areas of fibrosis and atrial muscle. Optical mapping is conducted to localize AF sources as described below and elsewhere.
Effect of Gal-3 Inhibition on Prevention of PAF-Induced Structural and/or Electrical Remodeling in the Atria of PAF Sheep.
A comparative placebo controlled trial galectin inhibitors is conducted to the effect of Gal-3on pro-fibrotic and electrical remodeling effects of TGF-acceleration and PAF perpetuation.

### In Vivo Study:

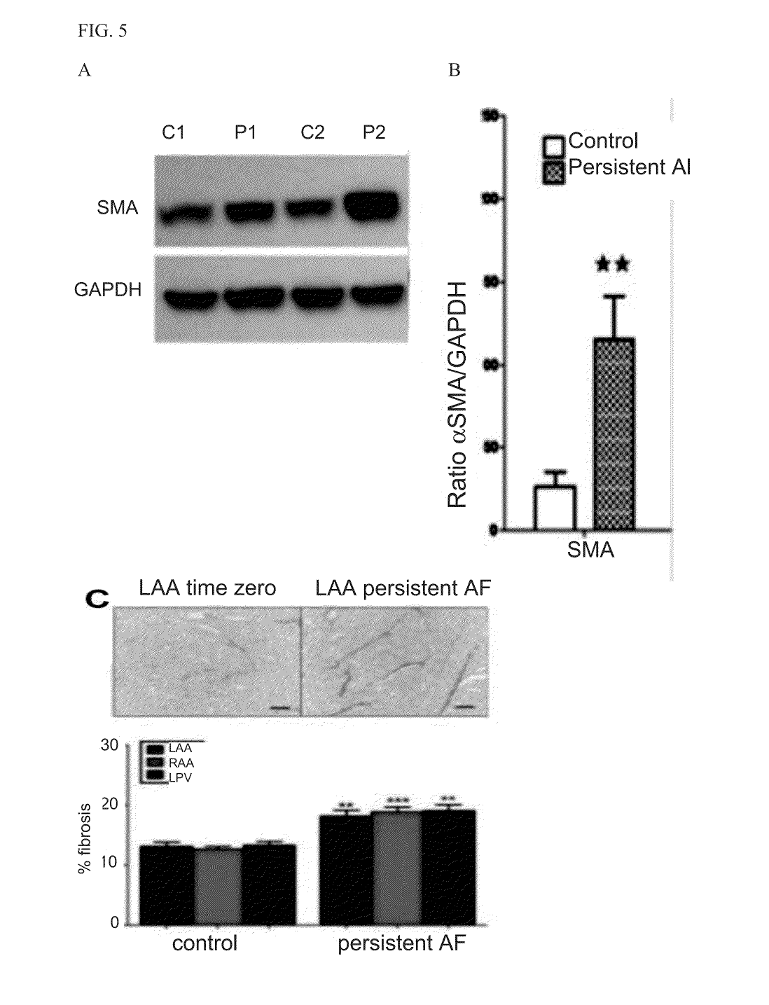

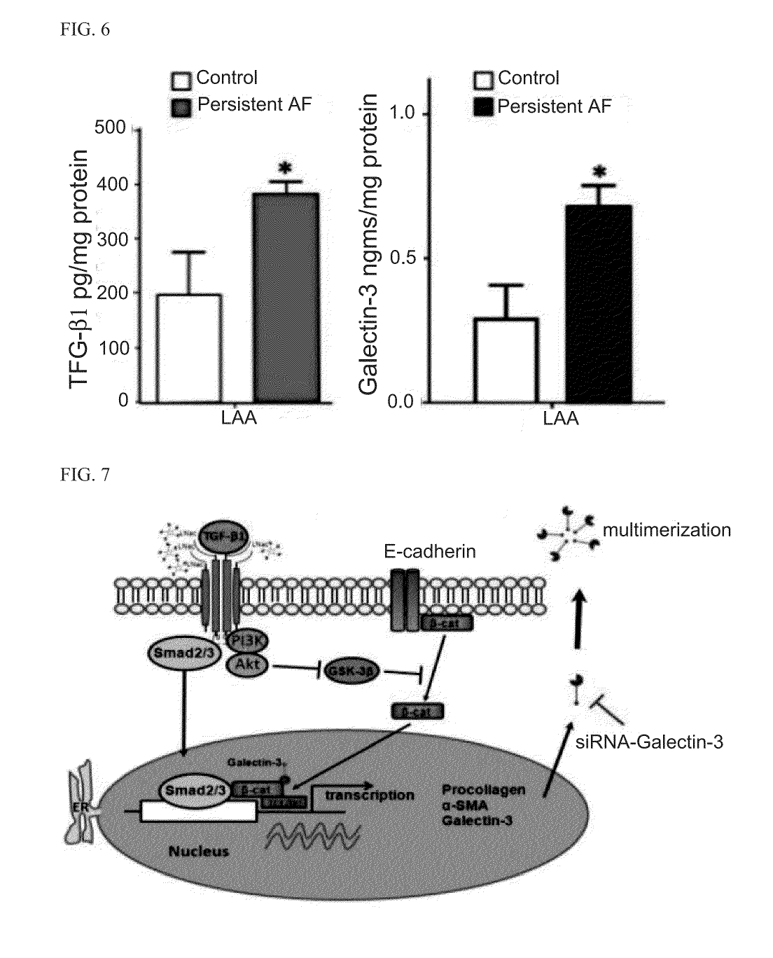

The surgical procedure and pacing protocol is described as follows: Under general anesthesia, sheep undergo implantation of a single-chamber pacemaker. A bipolar silicone lead is inserted into the right atrium (RA) under fluoroscopic guidance through the right external jugular vein. Once properly placed, the proximal end is screwed onto the sterile pacemaker. The pacemaker canister (single lead pacemaker from St Jude Medical, St Paul, Minn., USA) is inserted in a subcutaneous pouch at the base of the neck. In addition, a subcutaneous loop recorder (IRL; Reveal® XT, Medtronic) is placed on the left side of the sternum in close proximity to the left atrial free wall. This loop recorder monitors the heart rhythm for the appearance of AF. AF is induced by fast atrial pacing (20 Hz). A 6 to 30 sec pacing period is followed by a 10 sec sensing period in which the pacemaker is able to monitor the atrial rhythm. If AF is detected, pacing does not restart at the end of the sensing period. If sinus rhythm is detected, the pacemaker restarts the pacing protocol for 6 to 30 new seconds until the next sensing period. Paroxysmal AF episodes are expected to occur after ˜2-3 weeks of pacing and self-sustained persistent AF to develop after ˜9 weeks of pacing. In all sheep self-sustained persistent AF is maintained for 8-12 weeks. Electrograms are obtained from the intracardiac RA lead tip. The loop recorder is used to determine left atrial DF after QRS and T subtraction. The effects of GM-CT-01 will be compared with those of GR-MD-02 in-vivo and ex-vivo in a placebo controlled study. Sheep will be randomized to receive daily doses of GM-CT-01 (15 mg/Kg IV), GR-MD-02 (5 mg/Kg IV) or placebo for the duration of tachypacing (3 to 4 months). Blood samples are withdrawn every 7 days starting before the pacemaker is activated for drug concentration analyses as well as for determination of serum Gal-3 and TGF-β1, as well as fibrosis markers (as described in detail below and elsewhere (Yoshida et al., Heart Rhythm. 2011; 8:181-187). The primary endpoint is the number and duration of AF episodes after starting the pacing protocol/drug treatment. Secondary endpoints are α-SMA, Gal-3 and TGFβ1 mRNA (ELISA) and protein (Western blot) tissue levels, interstitial fibrosis (histology; see FIG. 5C) and the DF of AF as measured by FFT analysis of the RA and loop recorder data and the optical mapping study. The code is broken at the end of the in vivo study.

Echocardiographic Measurements: Echocardiograms are obtained in all animals at the outset and the end of the experiment. Left and right atrial diameters are measured during atrial diastole from both parasternal long-axis and short axis views (Vivid Q, General Electric, Inc). PICP, CITP, and PIIINP: Serum from all sheep are obtained prior to pacemaker implantation and weekly thereafter. All samples are obtained from a peripheral vein, and the serum is extracted and stored at −80° C. PICP (Takara Biomedical), CITP (Orion Diagnostica), and PIIINP (Usyn) enzyme immuno-assays are analyzed according to the manufacturer's specifications and measured at 450λ. RT-PCR: Described above (In vitro study).

Serum TGF-β1 and Gal-3: Quantification of TGF-β1 in samples is performed with the Quantikine Immunoassay, according to manufacturer's instructions (R&D systems, MN, USA). Gal-3 is measured by an ELISA kit (Bender Medsystems, Vienna, Austria) and measured on a plate reader. Calibration is according to the manufacturer's protocol. Values are normalized to a standard curve; intra-assay and inter-assay variances are determined to ensure low variability.

Ex-Vivo Study:

Optical mapping is conducted at the end of the in-vivo study in isolated hearts (Yoshida et al., supra). The dynamics of AF patterns and regional dominant frequencies in the hearts from animals receiving placebo are compared to those receiving daily doses of galectin inhibitor GM-CT-01 or GR-MD-02. Hearts are removed via thoracotomy and connected through the aorta to a Langendorff-perfusion system with re-circulating oxygenated (95% O2, 5% CO2) Tyrode's solution as described elsewhere (Berenfeld et al., Heart Rhythm. 2011; 8:1758-1765). After isolation, some hearts may not resume AF, which will enable measurements of atrial conduction velocity and its frequency dependence. Thereafter, the intracavitary pressure is increased, which provides a clinically relevant means to induce resumption and maintenance of AF (Yamazaki et al., Heart Rhythm. 2009; 6:1009-1017; Kalifa et al., Heart Rhythm. 2007; 4:916-924). Therefore, after an atrial trans-septal puncture all the vein orifices are sealed, except the inferior vena cava, which is cannulated and connected to a digital sensor and to an outflow cannula to control the intra-atrial pressure. The pressure is then be increased to 5 cm H₂O (normal diastolic LA pressure), and maintained throughout the experiment. Prior to sealing the veins, tetrapolar electrode catheters are placed in each of the PVs to record bipolar signals from the two distal electrodes (sampling rate, 1.0 kHz) using a Biopac Systems amplifier. Two additional custom-made bipolar electrodes are placed on the top of RAA and LAA. Epicardial and endocardial mapping of the LAA and PLA, respectively, are performed simultaneously. A bolus injection of 5 to 10 ml Di-4-ANEPPS (10 mg/mL) is administered before the acquisition. The emitted fluorescence from the epicardial surface of LAA is projected onto a CCD video camera (80×80 pixels, 600 frame/s). A second CCD camera is coupled to a rigid borescope through a custom-made eyepiece adapter (Yamazaki et al., Cardiovasc Res. 2012). The borescope is introduced through the anterior wall of the left ventricle, across the mitral valve and focused on the endocardial surface of the PLA. Two regions of the left atria are mapped simultaneously using the dual CCD camera system and the voltage sensitive dye Di-4-ANEPPS: LAA is mapped by a CCD camera focused on the epicardium; the endocardium of the posterior left atrium (PLA) is mapped. Patterns of wave propagation are determined using isochronal and phase mapping and the local frequencies are determined after fast Fourier transformation (FFT) of each pixel location (Yoshida et al., supra). The effects of galectin inhibitors on LA and RA DFs, APDs and wave propagation dynamics during AF are reported.

Statistical analyses: SPSS for Windows (SPSS Inc, Chicago, Ill.) is used. Continuous variables with normal distributions are expressed as mean±SD. Categorical variables are expressed as frequency (percentage). A value of p<0.05 is considered statistically significant.

Echocardiographic Measurements: Echocardiograms are obtained in all animals at the outset and the end of the experiment. Left and right atrial diameters are measured during atrial diastole from both parasternal long-axis and short axis views (Vivid Q, General Electric, Inc). PICP, CITP, and PIIINP: Serum from all sheep is obtained prior to pacemaker implantation and weekly thereafter. All samples are obtained from a peripheral vein, and the serum is extracted and stored at −80° C. PICP (Takara Biomedical), CITP (Orion Diagnostica), and PIIINP (Usyn) enzyme immuno-assays are analyzed according to the manufacturer's specifications and measured at 450λ. RT-PCR: Described above (In vitro study).

Serum TGF-β1 and Gal-3: Quantification of TGF-β1 in samples is performed with the Quantikine Immunoassay, according to manufacturer's instructions (R&D systems, MN, USA). Gal-3 is measured by an ELISA kit (Bender Medsystems, Vienna, Austria) and measured on a plate reader. Calibration is according to the manufacturer's protocol. Values are normalized to a standard curve; intra-assay and inter-assay variances are determined to ensure low variability.

### Example 5

### Methods

Adult cardiomyocytes and fibroblast isolation: Cardiomyocytes were isolated from normal adult male CD rats (200-300 g). Briefly, after quick removal, hearts were washed in ice-cold phosphate buffered saline (PBS), then retrogradely perfused through the aorta for up to 5 minutes with modified Krebs buffer (KHB) containing (in mM) NaCl 118, KC14.8, HEPES 25, K2HPO4 1.25, MgS04 1.25, glucose 11, CaCl2 1, pH 7.40. The perfusate was then switched to modified Krebs buffer without calcium for 3 minutes. Following calcium-free KHB perfusion hearts were digested by perfusing calcium-free KHB containing 200 units/ml collagenase II, (Worthington Biochemicals, Lakewood, N.J.) and blebbistatin (33.3 μM) for 15 min. The collagenase digested hearts were removed from the apparatus and atria were discarded. Ventricles cell suspension was centrifuged (500×g) for 30 sec, the cell pellet was resuspended in KHB-A containing 2% bovine serum albumin and blebbistatin. The cell suspension was centrifuged again and resuspended in culture media (Medium 119, Sigma) containing glutathione (10 mM), NaHCO₃ (26 mM), 100 units/ml penicillin, 100 μg/ml streptomycin and 5% fetal bovine serum. Cells were plated on laminin coated (40 μg/ml) tissue culture cover slips. After 2 hr, the medium was changed to serum-free MI99.
Cardiac fibroblast isolation: Ventricles cell suspension supernatant from both spins was saved for fibroblast isolation. The suspended fibroblasts were centrifuged at 2000 rpm for 10 min and the cell pellet was suspended in DMEM supplemented with 1% penicillin/streptomycin, and 10% fetal bovine serum (full medium). Cardiac fibroblasts were grown in this same full medium until 70-80% confluent and passaged using 0.05% trypsin EDTA.
Collection of fibroblast conditioned medium: Cardiac myofibroblasts at passage 3-5 were plated in 100-cm dishes (5×105). Cells were allowed to grow in full medium for one day. At the end of the growth period full medium was aspirated and cells were rinsed with Ca2+/Mg2+ free PBS and 10 ml of serum free medium was added to each dish. After 24 hr the conditioned medium was collected, filter sterilized and stored at −80° C. until further used.
Cytokine array and TGF-β1 ELISA: Cytokine array was performed using a commercially available Proteome Profiler Rat Cytokine Array Kit (R and D systems, Minneapolis, Minn.). Briefly array membranes were incubated with 2 ml of conditioned medium overnight in the cold room and the assay was performed according to manufacturer's instructions.

Levels of total TGF-β1 released in the culture medium were analyzed using commercially available Enzyme-linked immunosorbent assay kit (R and D systems, Minneapolis, Minn.). Briefly 2 ml of conditioned medium was activated with HCl, 100 μL, of activated conditioned medium was used in the TGF-β1 ELISA kits according to manufacturer's instructions. All assays were done in duplicate. Results are expressed as picogram of TGF-131/ml of media.

Cell Treatment: Isolated adult rat cardiac myocytes were treated with FCM or TGF-β1 (R and D systems, Minneapolis, Minn.) for 3 days in serum free medium. For PI3K pathway inhibition cells were pretreated (30 min) with 10 μM LY29004 (Cayman Chemicals, Ann Arbor) before the treatment with TGF-β1.
mRNA analysis by quantitative PCR (qPCR): Cardiac myocytes were washed with PBS and lysed with lysis buffer. RNA was isolated from the myocardial tissue using RNAeasy kit from Qiagen (Qiagen, Valencia, Calif.) according to the manufacturer's instructions. Isolated RNA from these samples was treated with DNase for 15 min at room temperature (Qiagen, Valencia, Calif.). 100 ng of DNA-free total poly-A tail RNA (mRNA) was first subjected to synthesis of cDNA using Oligo dT primers applying SuperScript III First-Strand Synthesis System from Invitrogen (Invitrogen, Carlsbad, Calif.) according to the manufacturer's instructions. cDNA from 20 ng of total RNA was then subjected to real-time RT-PCR using predesigned taqman probe primers specific for rat Scn5a (Rn00565502), Kcnip2 (Kchip2; Rn01411451) and kcnd2 (Rn01456260) (Applied Biosystems, California). No-template controls and no-RT controls were run during each experiment to detect any RNA and/or DNA contamination. Results are expressed as fold expression of gene of interest normalized to GAPDH expression in the sample.
Western blotting: Control and treated cardiac myocytes were washed in cold PBS, lysed directly in the modified loading buffer (25 mmol/l Tris.HCl; 150 mmol/l NaCl; 1 mmol/l EDTA; 4 mmol/l NaF; 2 mmol/l Sodium ortho-vanadate; 1% Triton X-100, protease inhibitor, 5% glycerol, 1% SDS, 0.05% bromophenol blue, 5% β mercaptoethanol) and sonicated. The lysate (20 μl) were then subjected SDS-PAGE as described earlier. The blots were incubated with rabbit pFOXO1 antibody, 1:500 (Cell Signaling) or rabbit GAPDH antibody, 1:5000 (Sigma-Aldrich, St. Louis, Mo.).
Patch-clamp experiments: Whole-cell ionic currents were recorded from adult rat ventricular myocytes. All recordings were conducted at room temperature. Sodium current recordings were conducted in a low-sodium extracellular solution containing (in mM): NaCl, 10; MgCl2, 1; CaCl2, 1.8; CdCl2, 0.1; HEPES, 20; CsCl, 127.5; glucose, 11. The pipette solution contained (in mM): NaCl, 5; CsF, 135; EGTA, 10; MgATP, 5; HEPES, 5. To characterize the voltage dependence of the peak INa, single cells were held at −120 mV, and 200 msec voltage steps were applied from −90 to +30 mV in 5 mV increments. The interval between voltage steps was 3 sec. Voltage-dependence of inactivation was assessed by holding cells at various potentials from −160 to −40 mV followed by a 30 msec test pulse to −40 mV to elicit INa. Recovery from inactivation was studied by holding cells at −120 mV and applying two 20-msec test pulses (S1, S2) to −40 mV separated by increments of 2 msec to a maximum S1-S2 interval of 80 msec. The S1-S1 interval was kept constant at 3 sec.

The extracellular solution for transient outward potassium current (Ito) contained (in mM): 136 NaCl, 4 KCl, 1.8 CaCl2, 2 MgCl2, 10 HEPES, 0.03 tetrodotoxin, 0.01 nifedipine, and 14 glucose, pH 7.35. The pipette solution contained (in mM):135 KCl, 1 MgCl2, 10 EGTA, 10 HEPES, 5 glucose, pH 7.2. Voltage-gated outward K+ currents were evoked during 5-s depolarizing voltage steps to potentials between −40 and +60 mV from a holding potential of −70 mV; voltage steps were presented in 10 mV increments at 15 s intervals

Action potentials were recorded from individual myocytes using the current clamp mode of the MultiClamp 700B amplifier after gigaseal formation and patch break. Stimulus pulses (1-2 ms duration) were generated using a World Precision Instruments DS8000 stimulator (Sarasota, Fla.). The bath solution contained (in mM): NaCl: 148, KCl: 5.4, MgCl2: 1, CaCl2 1.8, NaH2PO4: 0.4, HEPES: 15, Glucose: 5.5, pH 7.4 with NaOH. The pipette solution contained (in mM), KCl: 20, K-aspartate: 90, KH2PO4: 10, EDTA: 5.0, K2ATP: 1.9, HEPES: 5.0 and Mg2+ 7.9; pH 7.2 (KOH)

Statistical analyses: In all cases, “N” indicates the number of animals and “n” the number of experiments (e.g., FIG. 1). Comparisons of individual group means used a two-tailed Student's t test. One-way analysis of variance (ANOVA) with Bonferroni post-test was used to compare multiple data sets. All statistical calculations were done using GraphPad Prism version 5 (GraphPad Software Inc., San Diego, Calif.) and p<0.05 was considered significant. Data are presented as mean±standard error of the mean.

### Results

In all experiments, cells were exposed to one of the following for 72 hrs prior to the experiment: control medium, FCM or TGF-β1.

In control experiments the mean peak sodium current measured in freshly dissociated rat ventricular myocytes (Day 0) was similar to that measured after 72 hours in control medium (Day 3). At −45 mV, peak INa was −38.15±1.79 pA/pF in Day 0 cells (n=3) and −36.75±4.53 pA/pF in Day 3 cells. (n=5). This difference was not statistically significant, which agrees with previous work showing no difference in sodium channel properties between days 0 and 5 in culture (Sato et al., Circ Res. 2009; 105:523-526).

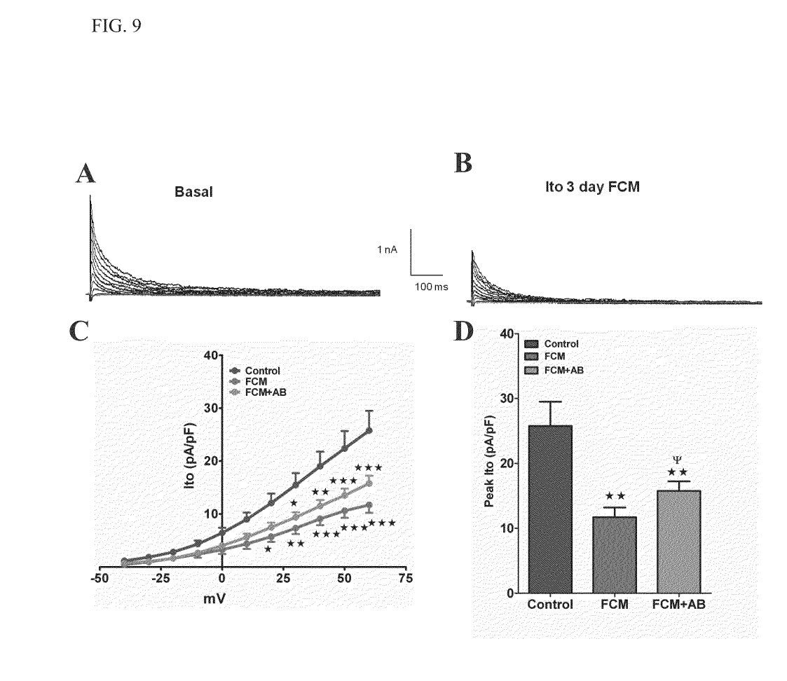

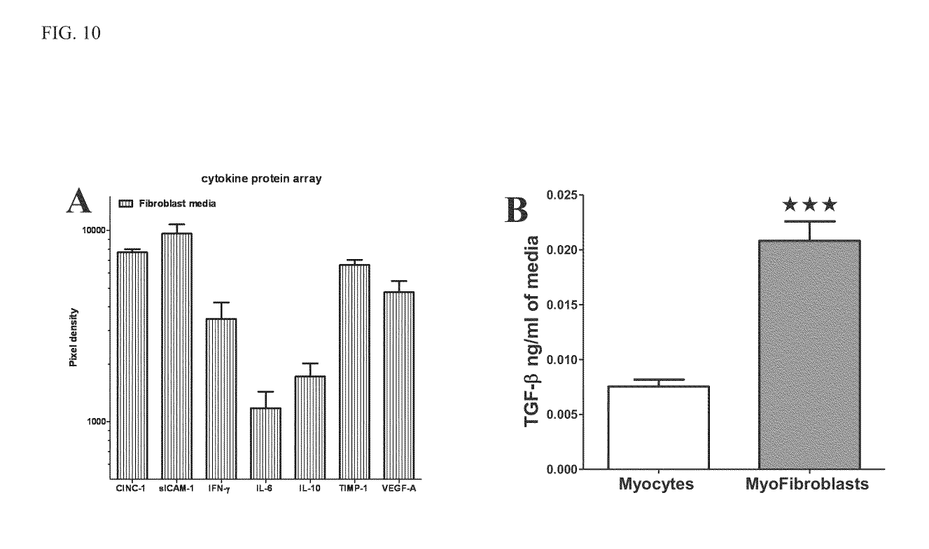

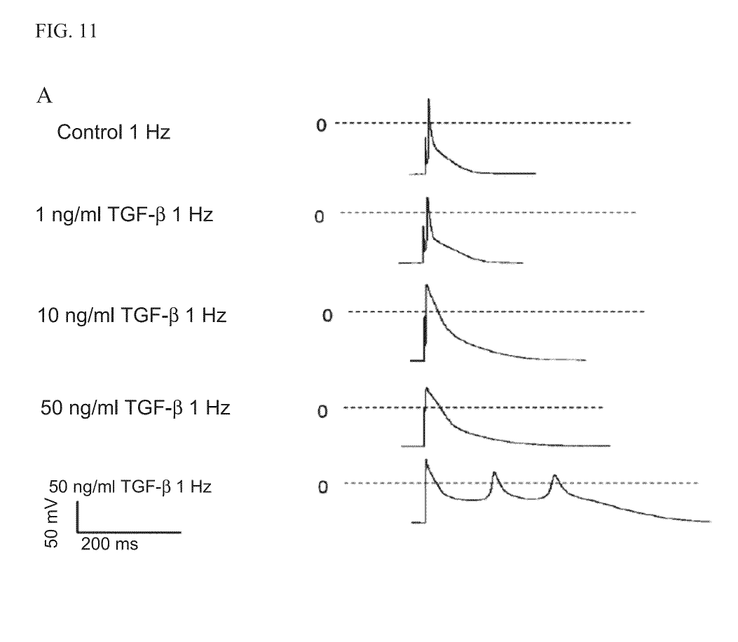

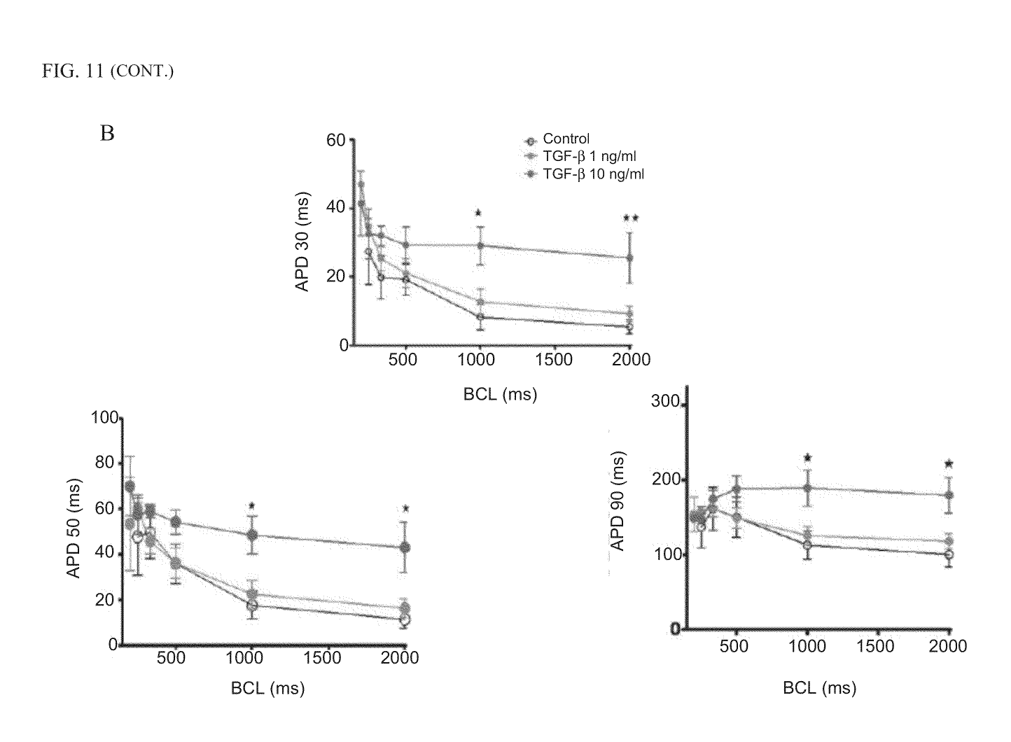

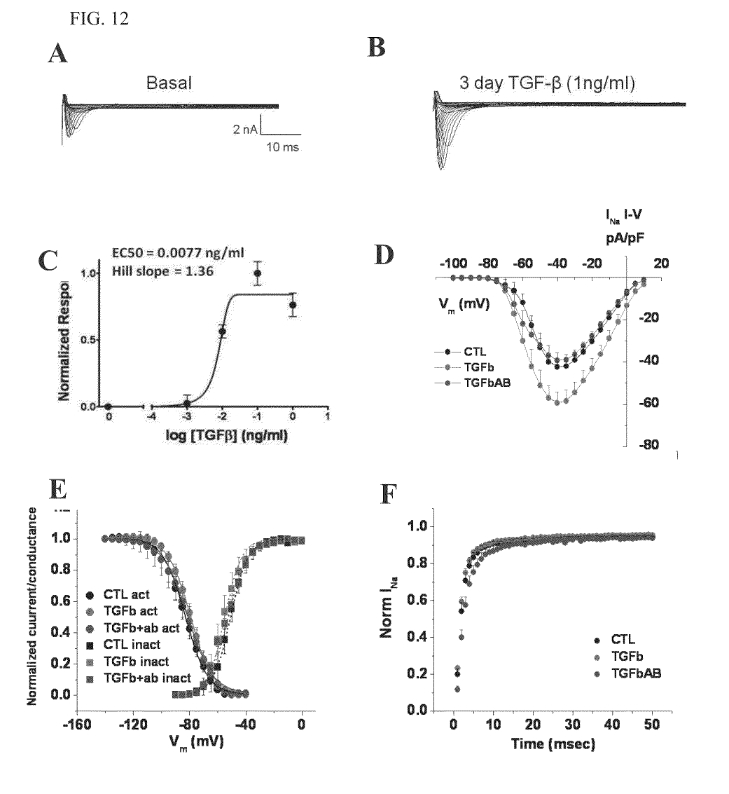

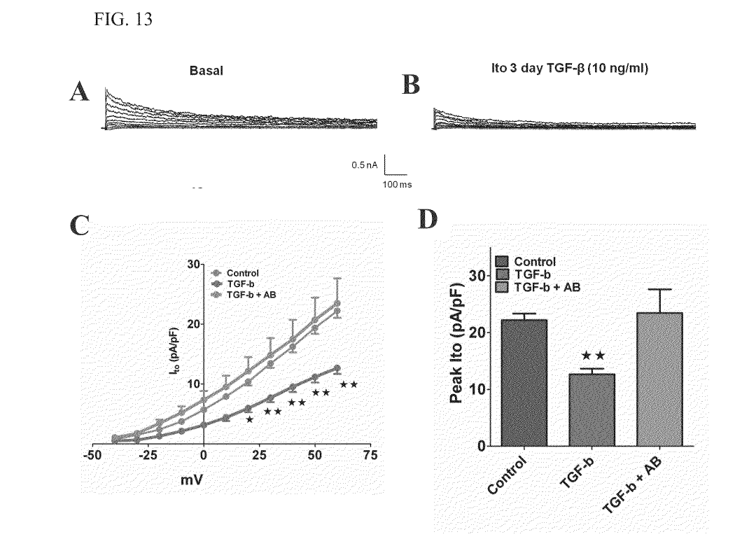

Exposure of ARVMs to FCM Increases Sodium Current: FIG. 8 shows data from whole cell-voltage clamp experiments in which INa was recorded at room temperature, in a low-sodium extracellular solution (Auerbach et al., J Physiol. 2011; 589:2363-2381) and holding potential (HP) of −120 mV; 200 msec voltage steps were applied from −90 to +30 mV in 5 mV increments every 3 sec. FIG. 8A shows representative superimposed INa traces obtained at varying voltages in control top panel and FCM lower panel INa for FCM was larger than control at all voltages. B shows superimposed mean INa current density-voltage (IV) relations. Compared to control, incubation for 3 days with FCM significantly increased peak current at voltages between −50 and −40 mV (p<0.01). Voltage-dependence of activation (m∞) and inactivation (h∞) was assessed by holding cells at various potentials from −160 to −40 mV followed by a 30 msec test pulse to −40 mV to elicit INa (Auerbach et al., J Physiol. 2011; 589:2363-2381). As illustrated in Panel D, neither the h∞ nor the m∞ curve was modified, indicating that FCM did not change the channel's biophysical properties.
Exposure of ARVMs to FCM Reduces the Transient Outward Current: Next, the effects of FCM on Ito were investigated to determine whether the downregulation of KV4.2 reported by other authors in neonatal rat myocytes (Cardiovasc Res. 1999; 41:157-165), as well as some heart failure models (Li et al., Am J Physiol Heart Circ Physiol. 2008; 295:H416-424), applies also to adult myocytes. FIG. 9 summarizes data conducted at room temperature. The top panels show representative superimposed Ito traces in cultured ARVMs obtained at varying voltages in the absence (A) and the presence of FCM (B). Three day exposure to FCM reduced Ito with respect to control at all voltages. Panel C shows, the superimposed Ito IV relations. Incubation of rat adult cardiac myocytes for 3 days with FCM (red) significantly decreased peak Ito at voltages between 20 and 60 mV compared to the control treated cells (black) (p<0.05-0.001). For example, at 60 mV current in control cells was 25.8±3.7 pA/pF whereas in FCM it was 11.7±1.4 pA/pF (p<0.001).
Cardiac myofibroblasts release active proteins in the culture medium: The unexpected results presented above indicate that cardiac fibroblasts introduced a soluble factor or factors into the medium that differentially altered INa and Ito current densities by either 1) a direct cell membrane or intracellular interaction; or 2) sequestration, consumption, or modification of factors in the standard medium, leading to an indirect biological effect (LaFramboise et al., Am J Physiol Cell Physiol. 2007; 292:C1799-1808). Thus, in order to address those questions, 1 ml FCM collected from fibroblasts harvested from normal rat hearts for analysis of specific cytokine proteins were assayed using a rat cytokine array kit. In FIG. 10A relative quantification of relevant cytokine proteins showed that CINC-1, sICAM-1, IFN-γ, IL-6, IL-10, TIMP-1 and VEGF were present at high levels in FCM as compared to myocytes conditioned media (FIG. 10A). Note that TGF-β1, whose active form measurement requires an acidic medium, is excluded from this array because acidification is likely to affect quantification of the other cytokines Thus, a rat TGF-β1 ELISA kit was used to determine TGF-β1 levels in FBS-free supernatant of cultured fibroblasts harvested from the ventricles of a normal rat. As shown in FIG. 10B the concentration of TGF-β1 in FCM was almost 3 times larger than that measured in myocytes conditioned medium (7.5±0.1 vs 20.8±1.7 pg/ml, p<0.01). All other cytokines shown in FIG. 10A were also detected in myocyte conditioned medium. But with the exception of CINC-1, their levels were significantly lower than in FCM.
FCM effects are inhibited by a neutralizing TGF-β1 antibody: Additional voltage clamp experiments were conducted to determine whether the differential FCM-induced changes in INa and Ito in our study depend, at least in part, on the TGF-β1 that is present at large concentration in that medium. Interestingly, as shown in FIG. 8B, 3-day incubation with FCM plus a TGF-β1 neutralizing antibody (FCM+, TGF-β1 ab), completely eliminated the FCM induced increase in peak INa current. Panel C shows that the recovery from inactivation was unaffected by the FCM or the antibody. On the other hand, as shown in FIG. 9, 3-day incubation with FCM plus a TGF-β1 neutralizing antibody (FCM+TGF-β1 ab blue) partially prevented the FCM effect on Ito. At 60 mV, current in FCM+TGF-β1 ab was 15.8±1.5 pA/pF, a value that was intermediate between control and FCM (p<0.05 when comparing FCM+TGF-β1 ab vs FCM alone).
TGF-β1 increases sodium current in adult cardiac myocytes: The results presented in the previous sections indicate that TGF-β1 is a major cytokine involved in the differential FCM-induced changes in INa and Ito. This was tested by conducting additional patch-clamp experiments to determine whether incubation of ARVM with exogenous TGF-β1 alone would modify INa and Ito densities. The superimposed current traces presented in FIGS. 11A and B were obtained from a representative experiment. 3-day exposure to 1 ng/ml TGF-β1 increased the current magnitude at all voltages. FIG. 11C shows a dose-response curve for peak inward INa density obtained when culturing myocytes for 72 hours in medium containing varying concentrations (0.001-1.0 ng/ml) of TGF-β1. A maximum INa increase of ˜40% was achieved at 1 ng/ml, which was also seen at 10 ng/ml. The calculated EC50 was 0.007 ng/ml; well below the TGF-β1 concentration in FCM (˜21 pg/ml; see FIG. 10B). As shown by the IV relation in panel D, 1 ng/ml TGF-β1 significantly increased the peak INa density at step voltages between −60 mV and −30 mV (p<0.05-0.01). At −40V the TGF-β1 treated cells had about 40% more inward current compared to cells treated with control medium (p<0.05). Most important, the TGF-β1 induced changes in INa peak density were completely abolished when cells were treated with TGF-β1 plus the neutralizing TGF-β1 antibody, demonstrating the specificity of the TGF-β1 signaling effects. Finally, as illustrated in FIG. 11E, neither the m∞ nor the h∞ curve was modified by TGF-β1 treatment.
TGF-β1 decreases Ito in adult cardiac myocytes: Ito was somewhat less sensitive than INa to the effects of TGF-β1. Therefore, 10 ng/ml was used for experiments. FIG. 12 shows that 72-hr exposure to 10 ng/ml TGF-β1 reduced the outward current to levels similar to FCM (see FIG. 9). In the presence of TGF-β1 Ito was significantly lower than control at voltages between +20 mV and +60 mV. At +20 mV TGF-β1 treatment reduced the current density by 42% (10.4±0.63 vs 5.9.4±0.67, p<0.05); at 60 mV, current density in TGF-β1 treated cells was at 57% of control levels (22.2±1.2 vs 12.6±0.98, p<0.001). The changes in Ito seen in TGF-β1 treated cells were completely prevented by co-treatment with the TGF-β1 neutralizing antibody (FIG. 12). A 72-hr exposure to 1 ng/ml TGF-β1 also significantly reduced the peak Ito but to a much modest level.
TGF-β1 increases APD in adult cardiac myocytes: From the substantial, yet contrasting effects of both FCM and TGF-β1 on the INa and Ito densities one would expect significant alterations in the action potential characteristics. As shown in FIG. 13, TGF-β1 (10 ng/ml) led to a basic cycle length (BCL) dependent action potential duration (APD) prolongation. For example, at a BCL of 1000 ms APD30 was >3.5 times larger in TGF-β1 treated cells compared to control (8.1±3.6 vs 29.1±5.6 ms; p<0.05). At 50 ng/ml TGF-β1, APD was so prolonged that some cells early after depolarizations (EADs). TGF-β1 leads to differential transcriptional regulation of channel protein genes.

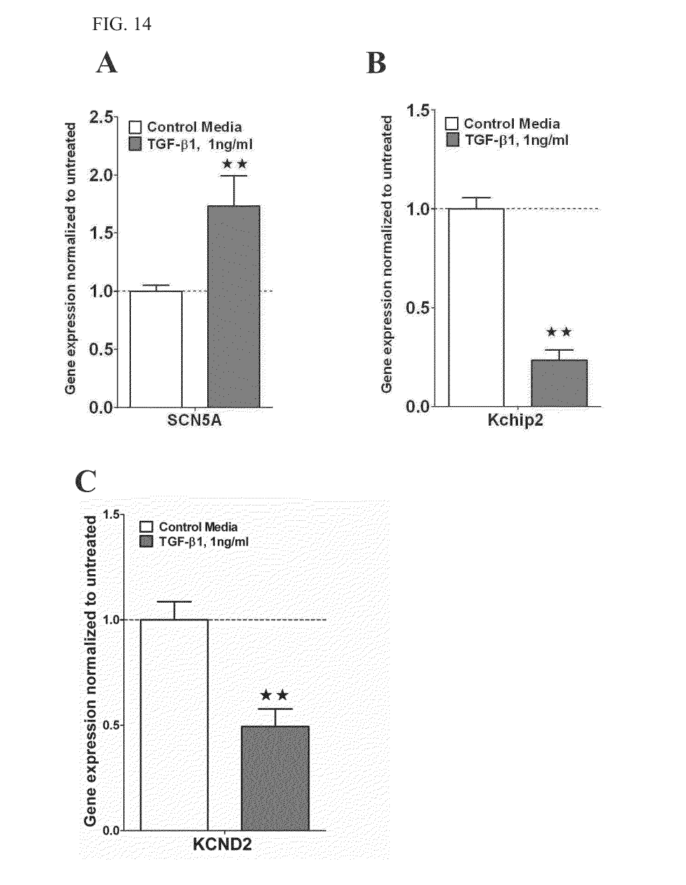

To investigate the molecular mechanism underlying the TGF-1 induced changes in INa and Ito densities, qPCR was performed on homogenates of isolated cells after 72 hr exposure to 1 ng/ml TGF-β1. This was the concentration that achieved maximum effect on the sodium current density (see FIG. 11C). As illustrated in FIG. 14A, in accordance with the increase in INa density, SCN5A transcript levels were significantly increased by 1.73±0.26 fold (p<0.01). On the other hand as shown in FIG. 14B, 1 ng/ml TGF-β1 significantly reduced mRNA levels of KCNIP2 by 77% (p<0.01). Moreover, in FIG. 14C, comparison of KCND2 expression in TGF-β1 treated cells showed a 50.6% decrease with respect to control (p<0.05).

The data indicate that the contrasting effects of TGF-β1 on INa versus Ito may be the result, respectively, of increased transcription and functional expression of SCN5A and reduced transcription of KCNIP2 leading to reduced KCND2 functional expression. Different signaling pathways mediate differential TGF-β1 effects on ion channel transcription Both NF-κB (Panama et al., Circ Res. 2011; 108:537-543) and the MEK/JNK pathways (Jia et al. Circ Res. 2006; 98:386-393) have been implicated in the regulation of KCNIP2 transcription. In addition, previous work supports a link between TGF-β1 and NF-κB signaling (Gingery et al., Exp Cell Res. 2008; 314:2725-2738). Those data point toward NF-κB signaling as a regulator of the TGF-β1 induced reduction in KCNIP2/KCND2 expression. Moreover, NF-κB has been implicated in the Ang II-induced decrease of SCN5A transcription and sodium current (Shang et al., Am J Physiol Cell Physiol. 2008; 294:C372-379). However, the data demonstrate that TGF-β1 increases NaV1.5 transcription and INa (FIGS. 11 and 14). Thus NF-κB is unlikely to have a role in NaV1.5 upregulation.

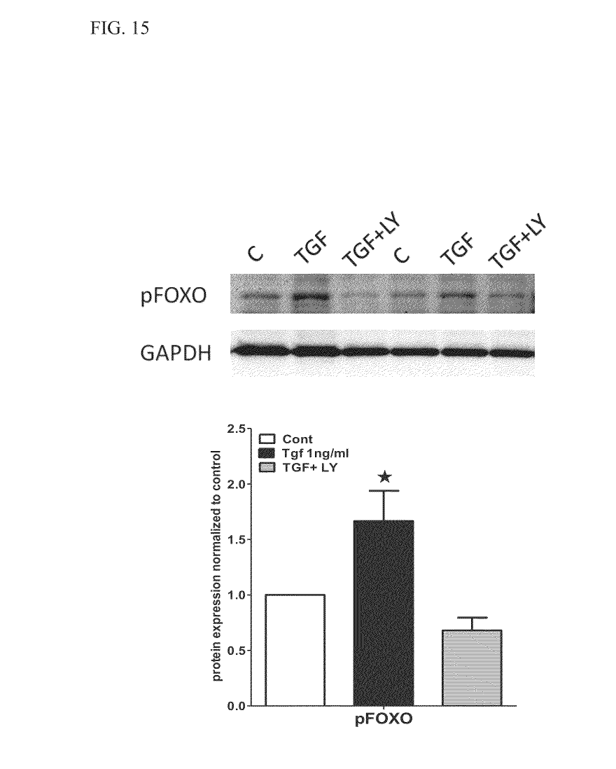

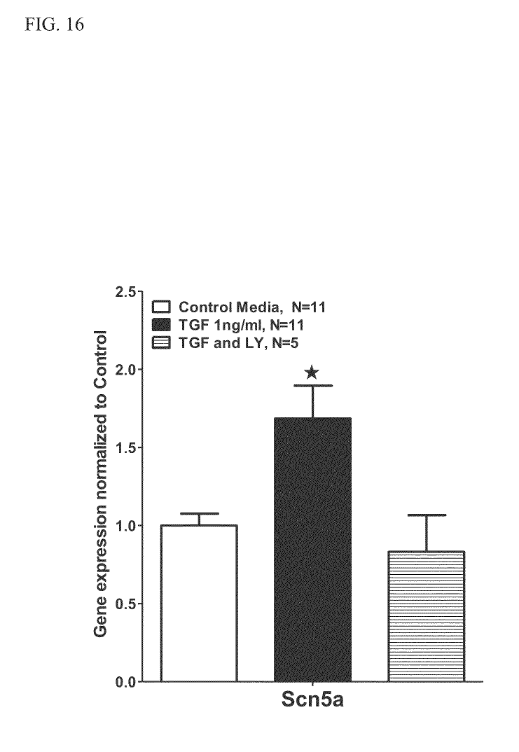

TGF-β1 Regulates SCN5A Expression via PI3K/Akt mediated phosphorylation of FOXO: It was hypothesized that in the adult rat myocyte the TGF-β1 induced increase in SCN5A transcription occurs via a direct interaction of TGF-β1 receptors with PI3K (Kato et al., J Am Soc Nephrol. 2006; 17:3325-3335; Carter et al., Curr Biol. 2007; 17:R113-114). PI3K acts on membrane phosphatidylinositol (PI) to generate the second messenger lipid PI-3-4-5-triphosphate, which recruits phosphatidylinositol-dependent kinase 1 and Akt kinase to the membrane (Seoane et al., Cell. 2004; 117:211-223). Then PI-dependent kinase-1 phosphorylates and activates Akt, which is known to phosphorylate several downstream proteins, including the Forkhead (FOXO) transcription factors, to control cell survival, cell growth and protein synthesis (Seoane et al., Cell. 2004; 117:211-223; Garcia et al., EMBO J. 2006; 25:655-661; Brunet et al., Cell. 1999; 96:857-868). A recent study has indicated that FOXO1 negatively regulates SCN5A transcription (Mao et al., PLoS One. 2012; 7:e32738) and effect that can be inhibited by Akt induced phosphorylation and translocation of FOXO1 from the nucleus to the cytoplasm. As shown in FIG. 15A TGF-β1 (1 ng/ml) induced phosphorylation of FOXO1 in adult rat cardiomyocytes at 5 min with peak at 15 minutes exposure. In FIG. 15B, a 67% increase in phosphorylated FOXO1 in TGF-β1 treated cells compared to the levels in control treated cells is shown. This increase in pFOXO1 was statistically significant (p<0.05). Moreover, phosphorylation of FOXO1 in TGF-β1 treated cells was inhibited in cells pretreated with PI3K inhibitor LY 29004. FIG. 15C demonstrates that the increased activation of PI3K was responsible for the TGF-β1-induced increase in SCN5A transcription. On the other hand, cardiomyocytes pretreated with the PI3K inhibitor LY 29004 failed to show increased SCN5A transcription by TGF-β1.

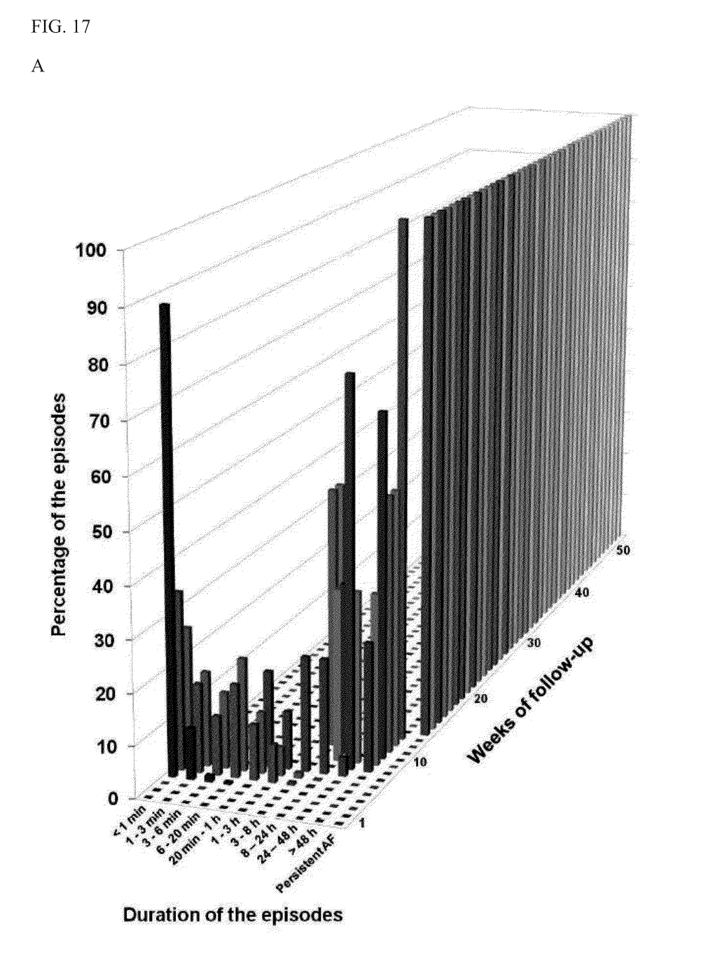

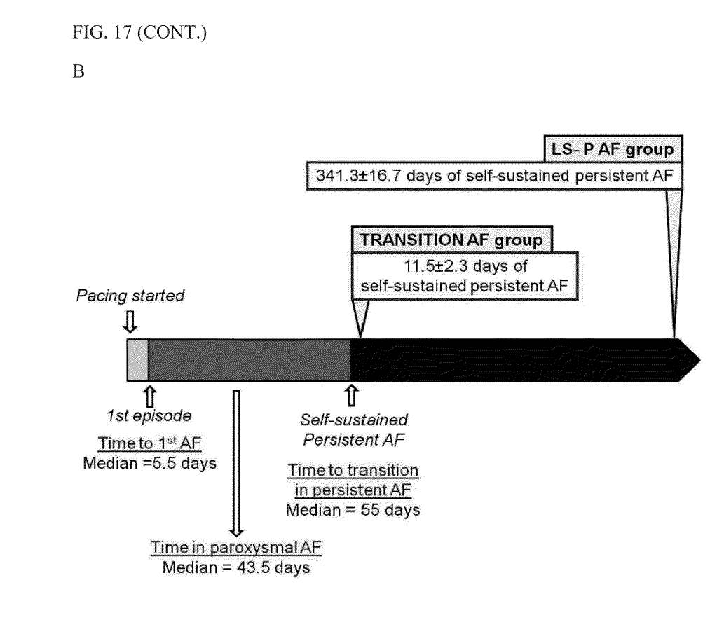

FIG. 16 shows morphologic and biochemical evidence for successful virally-mediated FOXO1-CA overexpression in adult rat cardiac myocytes. FIG. 17 shows the functional consequences of FOXO1-CA overexpression on the cardiac sodium current. As shown by the superimposed IV relation in panel A, FOXO1-CA significantly decreased the peak INa density at step voltages between −65 mV and −45 mV (p<0.05-0.01) compared with control treated cells. At −55 mV the FOXO1-CA treated cells had 43.8% less INa compared to cells treated with control treated (p<0.01). As shown in B, recovery from inactivation was not affected. Similarly, as shown in panels C, there was no change in the voltage dependence of either activation or inactivation. The effects of virally-mediated GPF expression alone, which decreased slightly, but not significantly the peak sodium current, and shifted the m∞ and h∞ curves somewhat in the depolarizing direction were also investigated.

### Example 6

A clinically relevant ovine model of intermittent right atrial (RA) tachypacing and demonstrated that after the first AF episode, dominant frequency (DF) of both the RA and left atrium (LA) increased gradually during a 2-week period, after which DF remained stable during follow-up (Filgueiras-Rama D, Price N F, Martins R P, Yamazaki M, Avula U M, Kaur K, Kalifa J, Ennis S R, Hwang E, Devabhaktuni V, Jalife J, Berenfeld 0. Long-term frequency gradients during persistent atrial fibrillation in sheep are associated with stable sources in the left atrium. Circ Arrhythm Electrophysiol. 2012; 5:1160-1167; herein incorporated by reference in its entirety).

The present example describes an ovine model where pacing stops temporarily when AF is initiated during paroxysmal episodes and permanently once AF is sustained without reverting to sinus rhythm (SR).

### Methods

### Pacemaker Implantation

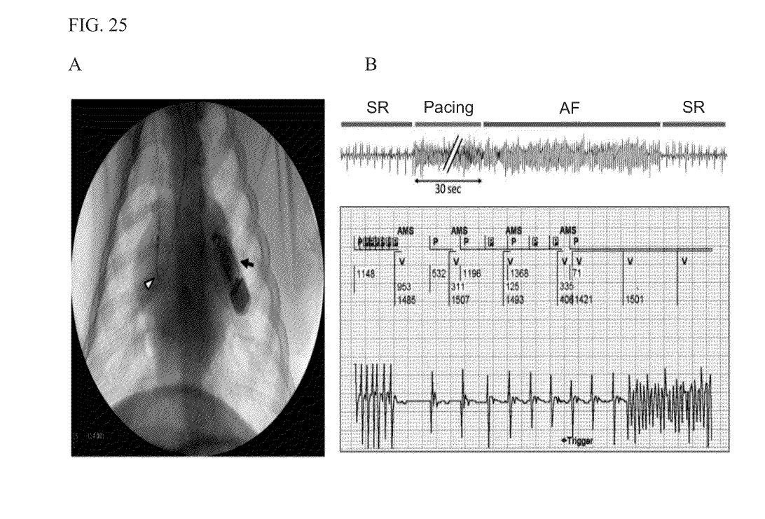

Procedures were approved by the University of Michigan Committee on Use and Care of Animals and complied with National Institutes of Health guidelines. Twenty-one 6-8 month-old sheep (≈40 kg) had a pacemaker implanted subcutaneously, with an atrial lead inserted into the RA appendage. Anesthesia was achieved using propofol IV for the induction (4-6 mg/kg) and isoflurane gas at 5-10 ml/kg for maintenance. An endocardial 6 to 8 Fr, bipolar, active fixation and steroid-eluting lead was inserted into the right atrial appendage through the left external jugular vein. Once properly placed, the proximal end was screwed to the atrial port of a sterile dual chamber pacemaker (St. Jude Medical, Inc, St Paul, Minn.). The ventricular port was occluded using specific plugs. The pacemaker canister was then inserted in a subcutaneous pouch at the base of the neck. In a subset of thirteen sheep (8 paced animals and 5 ones in SR), an implanted loop recorder (ILR, Reveal® XT, Medtronic, Inc. Minneapolis, Minn. USA) was placed subcutaneously on the left side of the sternum in close proximity to the LA (FIG. 25, Panel A).

### Pacing Protocol

After 10 days of recovery, sheep were assigned to either the Sham-operated group or to one of the atrial tachypacing groups. The sham operated animals had the device programmed in a sensing (OAO) mode only. Pacing voltage output was programmed at least twice the diastolic threshold for 0.4 ms duration to ensure appropriate atrial capture. The automatic mode switch (AMS) mode was enabled in the atrial tachypacing animals in order to avoid unnecessary pacing and allow AF to self-sustain once initiated (FIG. 25, Panel B). The AMS algorithm reliably generated tachypacing-induced self-sustained AF because the pacemaker resumed pacing only if AF stopped and sinus rhythm was detected. The pacemaker was programmed to induce AF by burst tachypacing; e.g., 30-sec pacing at 20 Hz at twice diastolic threshold followed by 10 sec sensing. The pacemaker resumed pacing only if AF stopped and SR was detected. In addition, devices had the capability of storing information on the history of AF, including the number and duration of AF episodes and the precise moment of each episode's occurrence. The Holter capabilities of the device were used to record intra-cardiac electrograms (EGMs) to accurately confirm the occurrence of AF, generate histograms and follow the evolution of AF. This was an attempt to reproduce the actual evolution of human AF, from the initiation of premature atrial beats, to paroxysmal and eventually persistent AF. Persistent AF was then defined as episodes lasting more than 7 days without reversal to sinus rhythm and necessity for resumption of pacing. Thus, in addition to the Sham-operated group (N=7) a subset of animals assigned to the fast atrial pacing group was sacrificed after 7 days of self-sustained AF (Transition group, N=7). The rest of the animals was sacrificed after one year of self-sustained AF (LS-PAF group, N=7). The ILR was programmed to identify AF episodes lasting >6 sec based on RR irregularity during the 10 sec sensing. Both pacemaker and ILR were interrogated weekly during the study period.

### Electrogram Acquisition and Processing

After group assignment, a weekly interrogation was performed. Persistence of sinus rhythm was verified in sham-operated animals and pacemaker memories were checked to detect if spontaneous episodes of AT/AF occurred. Three recordings were obtained in the tachypaced sheep during the follow-up: 1) RA lead tip EGM with a case reference; 2) Standard Lead I of the resting ECG exported at a 512 Hz sampling rate; and 3) ILR single lead recording (representing a LA far-field signal) exported as a vector PDF file. The EGM waveforms encoded in the PDF files were magnified and then digitized in a custom made Matlab program (MathWorks, Natick Mass.). The digitized signal was then superimposed on the original EGM image for visual inspection. If a miss-match was found, the cause was determined and adjustments made to correct them and ensure quality data. Recordings obtained by the ILR, whose canister is external to the LA, contain a mixture of atrial and ventricular activity. To analyze the atrial activity, the ventricular activity (dubbed QRST) was subtracted from the original recordings. A principal component analysis based AF estimation (PCA) was used for QRST removal. After QRST removal, a biased-free bidirectional Butterworth band-pass filter (4-35 Hz) was applied to each trace, as previously described1. The fast Fourier transform (FFT) was then used as previously described to extract the dominant frequency (DF) from 5 sec-long signals from the ILR and RAA electrograms. Finally, DF values from RAA and ILR electrograms were compared to identify in-vivo differences between the two regions in the left and right atria.

### Serum Measurements

Serum was obtained from all animals at baseline, after the initiation of paroxsymal AF (e.g., as soon as the first episode was detected), at the transition from paroxysmal to persistent AF and after 1 year of self-sustained LS-PAF maintenance. All samples were obtained from a peripheral vein, the serum extracted, and stored at −20° C. PIIINP (Biotang, Wlatham Mass.) levels were measured by enzyme linked immunosorbant assay according the manufacturers' specifications. The sensitivity (lower detection limit) for the assay was 12.5 ng/ml. All samples were run in duplicate and measured at 450 nm.

### Echocardiography

Echocardiograms were obtained in awaked sheep in the sternal recumbency position using a Vivid Q echocardiograph (GE Healthcare, Horten, Norway) at baseline, at the time of transition from paroxysmal to persistent AF and at the last follow-up for the LS-PAF group. LA and RA dimensions and areas, severity of mitral regurgitation, left ventricular ejection fraction (LVEF), end-systolic and end-diastolic diameters, and septal and posterior wall thickness were evaluated using standard criteria of the American Society of Echocardiography.

### Heart Removal and Cell Dissociation

After the end of the follow-up, hearts were quickly removed via thoracotomy and placed in a cold cardioplegic solution. LA and RA walls were removed, weighted and cut in three different portions. The posterior portion was used for molecular biology, middle portion was used for histology analysis and the anterior portion was used for cell dissociation. The posterior left atrium (PLA) was sectioned longitudinally and stored for subsequent histology and molecular biology analysis.

Cell isolation was performed as previously described (Escande et al., Am J Physiol. 1987; 252(1 Pt 2):H142-148). Left and right atrial samples for dissociation were transferred into a stock solution containing (in mM): NaCl (120), KCl (5.4), MgSO4 (5), Pyruvate (5), Glucose (20), Taurine (20), HEPES (20) and nitrilotriacetic acid (5). Tissue was chopped into chunks of about 1 mm3 with scissors. Chunks were stirred for 12 min in the above-mentioned solution at 37° C. oxygenated with 100% O2. Every 3 min, the tissue was transferred to a fresh solution by filtering solution through gauze. Chunks were then transferred to the calcium free protease digestion solution containing (in mM): NaCl (120), KCl (5.4), MgSO4 (5), Pyruvate (5), Glucose (20), Taurine (20), HEPES (20) and protease type XXIV (Sigma) for 45 min. After the end of the protease digestion, chunks were transferred to the collagenase digestion solution containing (in mM): NaCl (120), KCl (5.4), MgSO4 (5), Pyruvate (5), Glucose (20), Taurine (20), HEPES (20), CaCl2 (0.05) and collagenase type I (Worthington) for 2 digestion time points. At 15 and 30 minutes, the filtrate containing myocytes was decanted and centrifuged for 2 min at 500 rpm. Supernatant was aspirated and pellets resuspended in KB solution containing (in mM): L-Glutamic Acid (50), KOH (70), KCl (30), L-Aspartic Acid-K (10), KH2PO4 (10), MgSO4-7H2O (2), Glucose (20), Taurine (20), Creatine (5), EGTA (0.5) and HEPES (10). Myocytes were centrifuged a second time to aspirate supernatant, and resuspended in KB before use. Cell dimensions (length and width) were measured in the ICaL extracellular solution before the recordings of the currents at 40× from images recorded using a 40× oil-immersion objective lens (N.A. 1.30) attached to a Nikon Eclipse Ti inverted microscope.

### Western Blotting

Sheep LA and RA tissue samples were washed with protease inhibitors (Roche, protease inhibitor tablet) containing PBS and flash frozen in liquid nitrogen. Frozen tissue (50-100 mg) was homogenized in 1 ml of lysis buffer containing (in mM): Tris.HCl (25), NaCl (150), EDTA (1), NaF (4), Sodium ortho-vanadate (2), Triton X-100 1% and protease inhibitor. The homogenate was centrifuged at 10000 rpm for 5 minutes; the supernatant was used for western blotting. The tissue lysates (20 μg) were then subjected to one-dimensional sodium dodecyl sulfate polyacrylamide gel electrophoresis. The blots were incubated overnight in cold room with one of the following antibodies, mouse monoclonal α-SMA (1:2000); rabbit GAPDH antibody (1:5000) both from Sigma-Aldrich, St. Louis, Mo.; rabbit CaV1.2 antibody (1:500); rabbit NaV1.5 antibody (1:500); rabbit KV11.1 antibody (1:1000); rabbit KV4.2 antibody (1:250) all from Alomone Labs, PO Jerusalem, IL; rabbit Col III antibody (1:1000) from Abcam Cambridge, Mass.; mouse monoclonal Kir2.3 (1:250) from NeuroMab, Davis, Calif. The protein bands were visualized using enhanced chemiluminescence (Thermo Scientific, Rockford, Ill.). For Ca²⁺⁻handling proteins, Mouse monoclonal Na+/Ca²⁺ exchanger (NaCX-1) (1:1000) and mouse monoclonal Phospholamban antibody (1:1000) were purchased from Millipore, Calif. Mouse monoclonal ryanodine receptor 2 (RyR2:4000) and mouse monoclonal SERCA2 ATPase antibody were purchased from Pierce Biotechnology, IL. Rabbit monoclonal RYR2 2814 Phospho Serine antibody (1:1000) was purchased from Badrilla Ltd. United Kingdom.

### Real-Time RT-PCR

Sheep left and right atrial samples were washed in RNase/DNase free ice cold PBS. Clean samples were preserved in RNA stabilizing agent (Ambion, Austin, Tex.) and stored at −80° C. till further use. RNA was isolated from the myocardial tissue using RNAeasy kit from Qiagen (Qiagen, Valencia, Calif.) according to the manufacturer's instructions. Isolated RNA from these samples was treated with DNase for 15 min at room temperature (Qiagen, Valencia, Calif.). 2 μg of DNA-free total poly A tail RNA (mRNA) was first subjected to synthesis of cDNA using Oligo dT primers using SuperScript III First-Strand Synthesis System from Invitrogen (Invitrogen, Carlsbad, Calif.) according to the manufacturer's instructions. cDNA from 20 ng of total RNA was then subjected to RT-PCR using sybergreen real time PCR master mix (Qiagen, Valencia, Calif.). For real time PCR sheep specific primers were designed using predicted sequences (Table 4). No-template controls and no-RT controls were run during each experiment to detect any RNA and/or genomic DNA contamination.

### Patch-Clamp Recordings

Ion currents and action potentials were recorded in the whole-cell patch-clamp configuration using a MultiClamp 700B amplifier and Digidata 1440A digitizer (Molecular Devices, Sunnyvale, Calif.). Patch pipettes had resistances of 2-6 MΩ when filled with intracellular pipette solution and placed in extracellular solution. After formation of a GΩ seal, the patch membrane was ruptured and cell capacitance (Cm) was determined by integration of capacitive transients elicited by 10-mV hyperpolarizing and depolarizing steps (10 ms duration) from a holding potential of −80 mV. Data acquisition and analysis was performed using pCLAMP software (ver. 10.3; Molecular Devices, Sunnyvale, Calif.). Current amplitudes were divided by Cm and expressed as current densities (pA/pF) to normalize for variable cell sizes. L-type calcium current (ICaL) was recorded with pipette solution containing (in mM): CsCl (120), TEA-Cl (20), MgCl2 (1), Mg-ATP (5.2), HEPES (10), EGTA (10), adjusted to pH 7.2 with CsOH; and extracellular solution containing (in mmol/L): TEA-Cl (148), NaH2PO4 (0.4), MgCl2 (1), glucose (5.5), CsCl (5.4), CaCl2 (1.8), HEPES (15), adjusted to pH 7.4 with CsOH. Activation of ICaL was elicited by 300-ms voltage steps from a holding potential of −50 mV. Amplitude of ICaL was measured as the difference between peak inward current and current at the end of the voltage step.

Sodium currents (INa) were recorded at room temperature (20-22° C.) with pipette resistances <2.8 MΩ when filled with pipette filling solution containing (in mM): NaCl (5), CsF (135), EGTA (10), MgATP (5), Hepes (5), pH 7.2. The extracellular bathing solution contained (in mM): NaCl (5), MgCl2 (1), CaCl2 (1.8), CdCl2 (0.1), glucose (11), CsCl (132.5) and Hepes (20); pH was maintained at 7.4 with CsOH at room temperature. Appropriate whole-cell capacitance and series resistance compensation (≧60%) was applied along with leak subtraction. To assess the INa density, cells were held at −160 mV and stepped to various test potentials from −100 to 30 mV in 5 mV increments, with 200 ms duration pulses and 2800 ms interpulse intervals. Voltage-dependent activation of INa was assessed by generating conductance voltage relationships (m-infinity curves) and fitting the data with a standard Boltzman function (Origin 8.1, Northampton, Mass., USA). Voltage-dependence of inactivation was assessed by holding the cells at −160 mV followed by a 300 ms test pulse from −140 to −40 mV in 5 mV increments; interpulse interval was 2700 ms. Recovery from inactivation was studied by holding cells at −160 mV and applying two 20 ms test pulses (51, S2) to −45 my, separated by increasing increments of 1 ms to a maximum S1-S2 interval of 50 ms. The S1-S1 interval was kept constant at 2000 ms.

The conventional whole-cell recording technique was employed to record the transient outward K+ current (Ito). Electrophysiological recordings were conducted at room temperature. The bath solution contained (in mM): NaCl (136), KCl (4), CaCl2 (1.8), MgCl2 (2), HEPES (10), tetrodotoxin (0.03), nifedipine (0.005), pH adjusted at 7.4 with NaOH. Recording pipettes contained (mM): KCl (135), MgCl2 (1), EGTA (10), HEPES (10), glucose (5), pH adjusted at 7.2 with KOH. Borosilicate glass electrodes were pulled with a Brown-Flaming puller (model P-97), yielding appropriate tip resistances when filled with pipette solution <3 MΩ. Appropriate whole-cell capacitance and series resistance compensation (≧70%) was applied. Leakage compensation was not used. Ito was record using a step protocol with a holding potential of −70 mV and stepping from −40 to +60 mV in 10 mV increments of 5 s at each potential every 20 s. Ito was measured as the difference between the peak current and the current at the end of the 5 s pulse.

Inward rectifier current (HU) was recorded with pipette solution containing (in mM): KCl (148), MgCl2 (1), EGTA (5), HEPES (5), Creatine (2), ATP (5), Phosphocreatine (5); adjusted to pH 7.2 with KOH and extracellular solution containing (in mM): NaCl (148), NaH2PO4 (0.4), MgCl2 (1), Glucose (5.5), KCl (5.4), CaCl2 (1), HEPES (15), pH adjusted at 7.4 with NaOH. Activation of XI was elicited by a step protocol utilizing 400-msec steps ranging from −120 to +20 mV in 10 mV increments with a holding potential of −50 mV and with 2 seconds between successive steps. 5 μM nifedipine was added to block ICaL channels and the Ca2+-sensitive ICl. BaCl2 (1 mM) was used to isolate IK1 from other background currents. Action potentials were elicited using square wave pulses (30-50 pA amplitude, 10-30 ms duration) generated by a DS8000 digital stimulator (World Precision Instruments, Sarasota, Fla.) and recorded at 37° C. with pipette solution containing (in mM): MgCl2 (1), EGTA (1), KCl (150), HEPES (5), phosphocreatine (5), K2ATP (4.46), b-hydroxybutyric acid (2), adjusted to pH 7.2 with KOH; and extracellular solution containing (in mM): NaCl (148), NaH2PO4 (0.4), MgCl2 (1), glucose (5.5), KCl (5.4), CaCl2 (1), HEPES (15), EGTA (1), pH adjusted at 7.2 with NaOH.

APD rate adaptation was analyzed by steady state stimulation at progressive shorter cycle lengths (CL) starting at 1000 ms, decreasing the CL slowly by 100 ms steps down to 300 ms and then by 20 ms steps after 300 ms cycle length. Action potentials at 1000, 500 and 300 ms CL were plotted in the rate adaptation curve.

### Histology

Tissue samples were sectioned longitudinally to the atrial wall plane at 4 μm, fixed in 10% buffered formalin, embedded in paraffin, and stained with picrosirius red. Patchy and interstitial fibrosis was quantified in both atria and in the PLA at 10× and 20× magnifications, respectively, using the BioQuant software (Bioquant Image Analysis Corporation, Nashville, Tenn.). A minimum of 20 randomly selected pictures per slide were analyzed by a blinded investigator, carefully excluding endocardial, epicardial and peri-vascular regions.

### Computer Simulations

Modified versions of the Grandi-Pandit4 model of the normal human atrial cell were used to simulate the cardiac action potential and its robust propagation in 2D cardiac tissue. The formulation for the fast sodium current in the original model was replaced with that of a mammalian ventricular myocyte model (Grandi et al., Circ Res. 109(9): 1055-1066) to achieve propagation in 2D cardiac tissue. In addition, the maximum conductance value for the inward rectifier potassium current, IK1, was increased by 30% to achieve tissue excitability and smooth propagation. The conduction velocity in the tissue was adjusted to 0.58 m/s (Gelband et al., Circ Res. 1972; 30(3):293-300; Workman et al., Cardiovasc Res. 2001; 52(2):226-235).

Atria in SR (equivalent to the Sham group) and at the transition stage from paroxysmal to persistent AF (equivalent to the Transition group) were simulated by modifying the magnitudes of INa, ICaL, Ito, and IK1 as observed in the experiments (listed in Table 5). Paroxysmal AF was simulated by incorporating all ionic changes similar to that in transition AF, except for ICaL, whose density was reduced by 30% only, such that the APD90 has values approximately half way in between SR and transition AF. See Tables 5 and 6). The steady-state cardiac action potentials were obtained by pacing the models for 50 seconds at 1 Hz. In all cases, reentry in 2D sheets (6 cm2) was initiated using the S1-S2 cross-field protocol.

### Statistical Analyses

Normally distributed data are expressed as mean±SEM. Normality of distributions was assessed using the Shapiro-Wilk test. A mixed regression model was applied to multiple group analyses and repeated measured data. Action potential durations (APD) and ionic current densities were compared using a two tailed unpaired Student's-t tests. RT-PCR and Westerns blot data were analyzed using two-way ANOVA. A p<0.05 was considered statistically significant.

### Results

### Sheep Model of Persistent AF

Of 21 implanted sheep one sham-operated animal was excluded and sacrificed prematurely due to severe symptomatic systemic infection. No atrial arrhythmias occurred in any sham-operated animals during follow-up. Also, no tachypaced animals developed signs of heart failure or stroke. FIG. 17 summarizes the time-course of AF development. The representative 3D plot (FIG. 17A) relates percentage of AF episodes in a given day (Y-axis) to duration of episodes (X-axis) and weeks of follow-up (Z-axis). The first AF episode occurred after a median time of 5.5 days after initiation of pacing (mean, 15.0±5.9 days; range, 0-62 days, FIG. 17B). AF episodes were then paroxysmal (<7 days duration), reaching self-sustained persistent AF (>7 days without reversal to SR) after a median of 43.5 days (mean 73.2±23.0 days; range, 19 to 346 days). Once in persistent, there was no further tachypacing as AF was detected uninterruptedly. Sheep in Transition and LS-PAF were sacrificed 11.5±2.3 days and 341.3±16.7 days, respectively, after occurrence of self-sustained persistent AF (i.e. after the last occurrence of SR).

### Persistent AF Leads to Atrial Dilatation

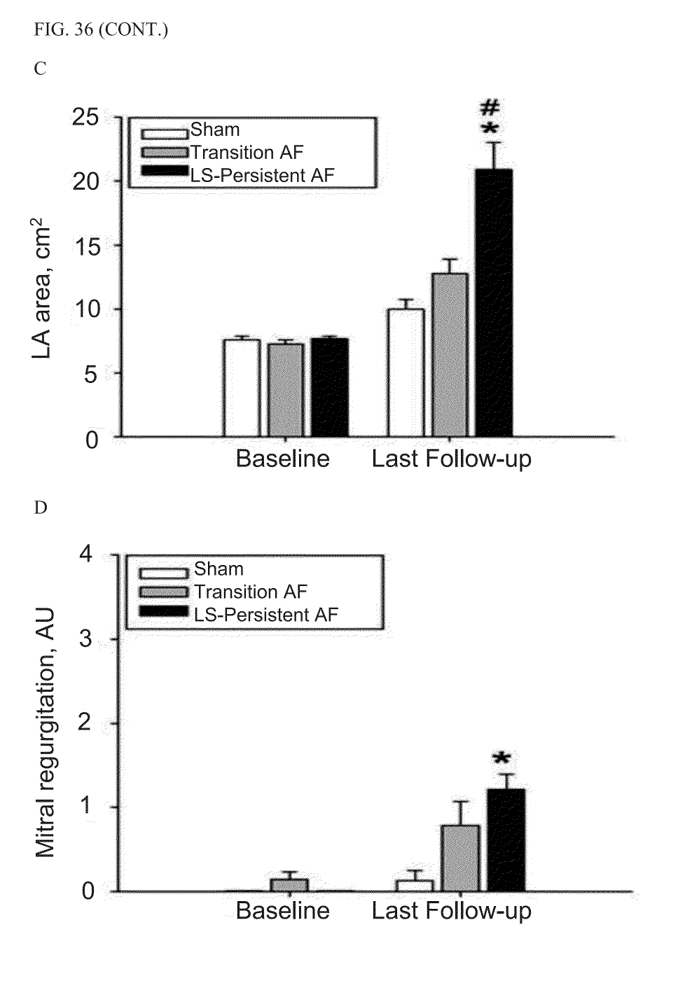

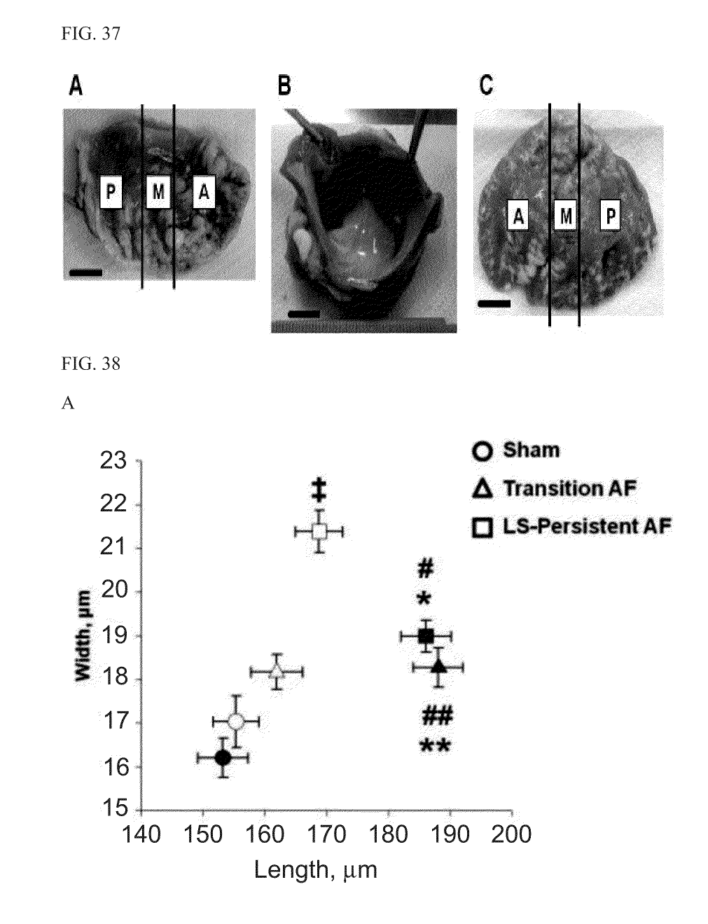

Echocardiographic findings (Table 1; FIGS. 35 and 36) revealed that LVEF was unchanged whereas RA and LA areas increased significantly in LS-PAF, compared with sham-operated and Transition groups (p<0.05; FIG. 36). At last follow-up, LS-PAF animals showed significant mitral valve regurgitation (FIG. 36B), yet LV end-diastolic volume, LV end-systolic volume or wall thickness were unchanged, ruling out tachycardia-induced cardiomyopathy associated with AF. Although, compared to sham-operated animals, the dry weight of isolated atria in the transition group tended to be larger, only the atrial tissues from the LS-PAF group demonstrated a significant increase weight (Table 2).

### Persistent AF Leads to Atrial Myocyte Hypertrophy

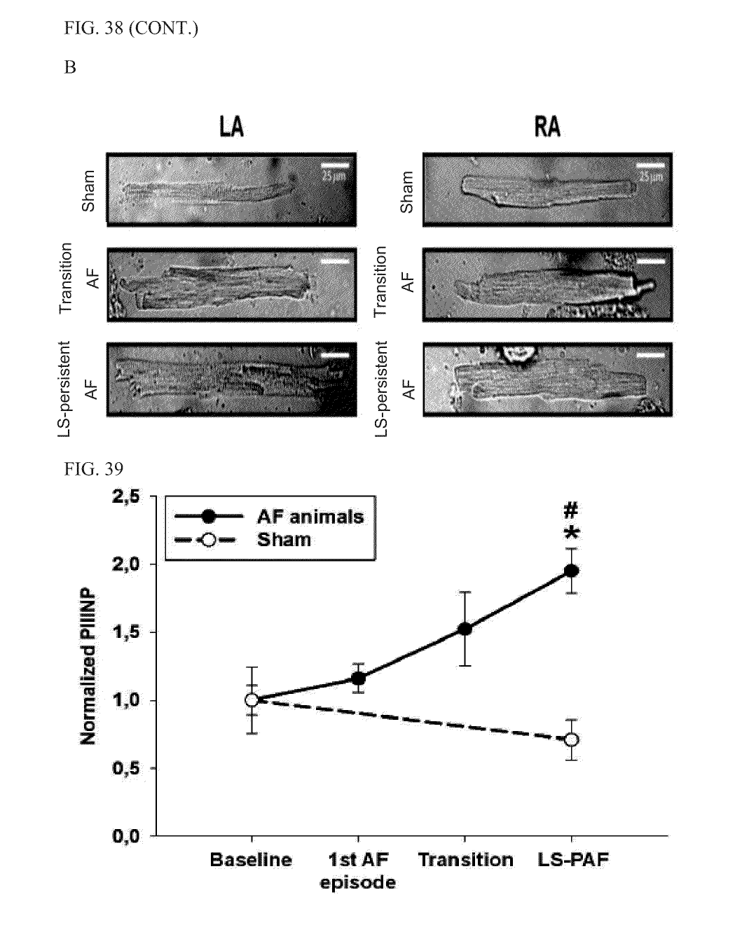

Mean LA and RA myocyte length and width, respectively, were similar for sham-operated animals (FIG. 38). At transition, LA myocyte length and width increased significantly (p<0.001 and p<0.01, respectively); RA myocyte length did not change significantly (p=0.25) and a trend for wider cells was observed (p=0.08). At transition, LA cells were longer than RA cells (p<0.001), and after one year of AF, no further differences were observed for LA myocyte lengths or widths compared to transition. However, RA myocytes that initially did not exhibit significant changes at transition, showed a trend for longer cells and were significantly wider (p<0.001). In LS-PAF, LA myocytes were longer (p=0.002) and thinner (p=0.001) compared to RA.

### AF Leads to Atrial Myofibroblast Activation and Fibrosis in the Absence of Heart Failure

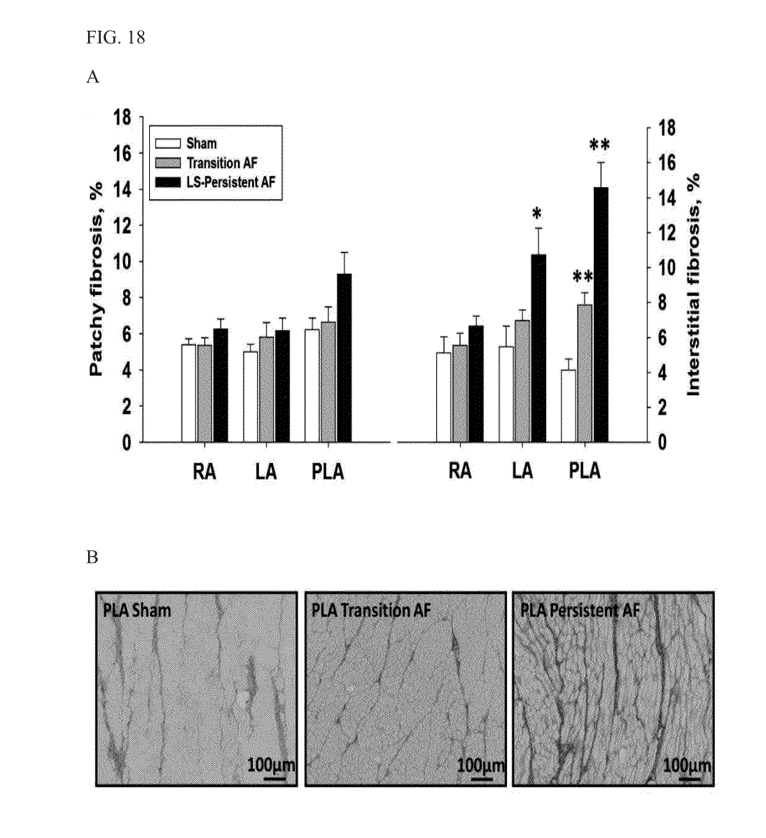

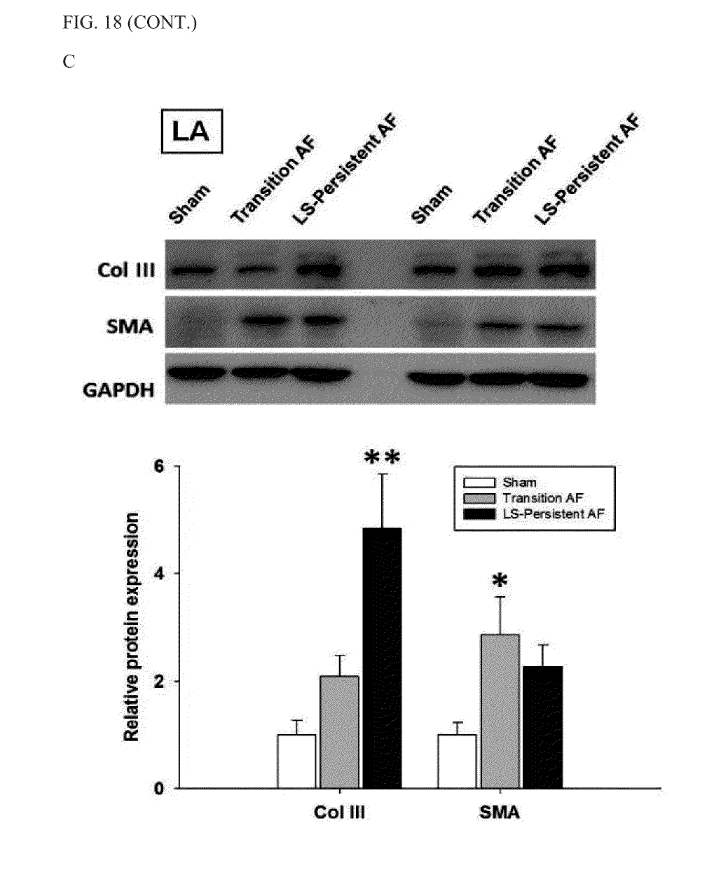

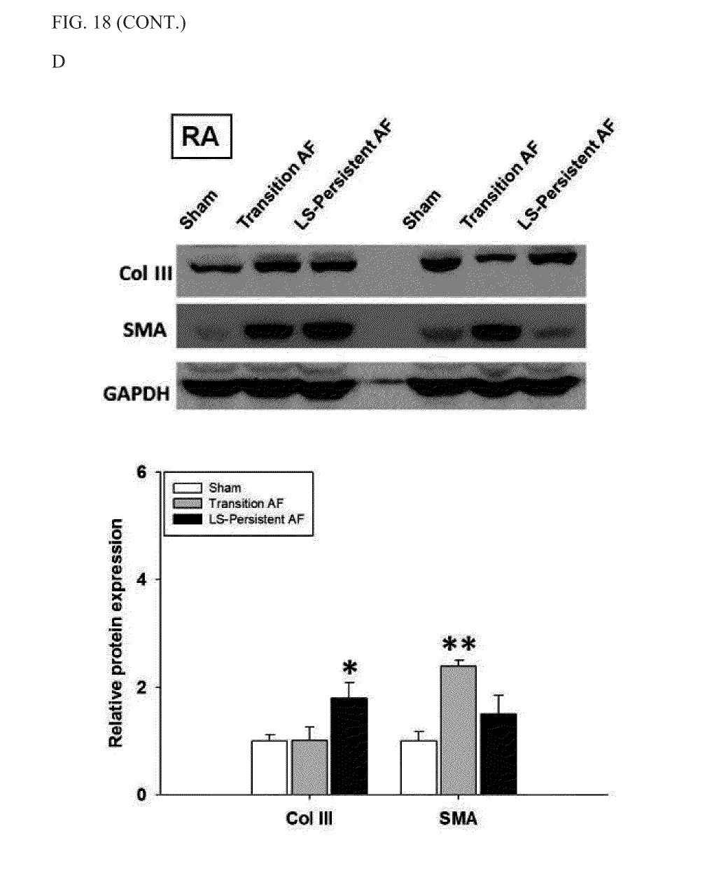

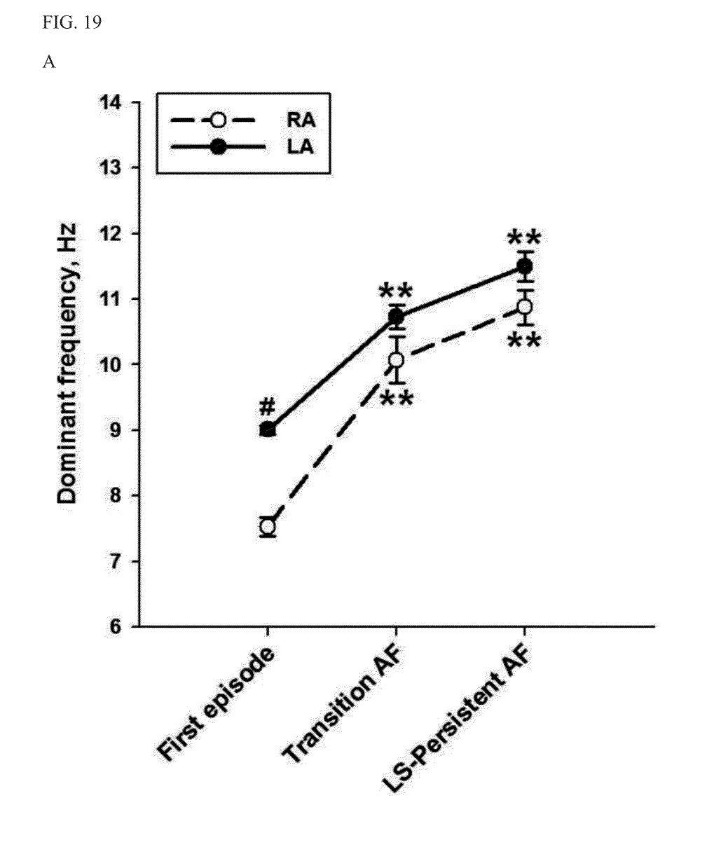

AF-induced changes in the extracellular matrix were analyzed using histology, serum markers and molecular biology. There was a trend towards increased patchy fibrosis in RA, LA and PLA regions during AF progression, interstitial fibrosis increased in both LA (from 5.5±1.2 to 10.7±1.5%, p<0.05) and PLA (from 4.1±0.6 to 14.6±1.4%, p<0.001), particularly in LS-PAF lengths (FIG. 18A-B, Table 3). These data correlated with measurements of PIIINP, a serum marker for collagen synthesis, which increased progressively, reaching maximal levels in LS-PAF which was increased significantly from Sham-operated animals at a similar time point (p=0.001 vs. sham; FIG. 39). Tissue protein levels of collagen III, analyzed by western blot, increased significantly in both atria during LS-PAF (FIGS. 18C and D). A significant increase in atrial α-smooth muscle actin (α-SMA), a marker of myofibroblast activation (Frangogiannis et al., Cardiovasc Res. 2000; 48:89-100) was seen in both atria in Transition, but decreased toward control levels in LS-PAF.

### Electrophysiological Remodeling is Reflected by DF Changes

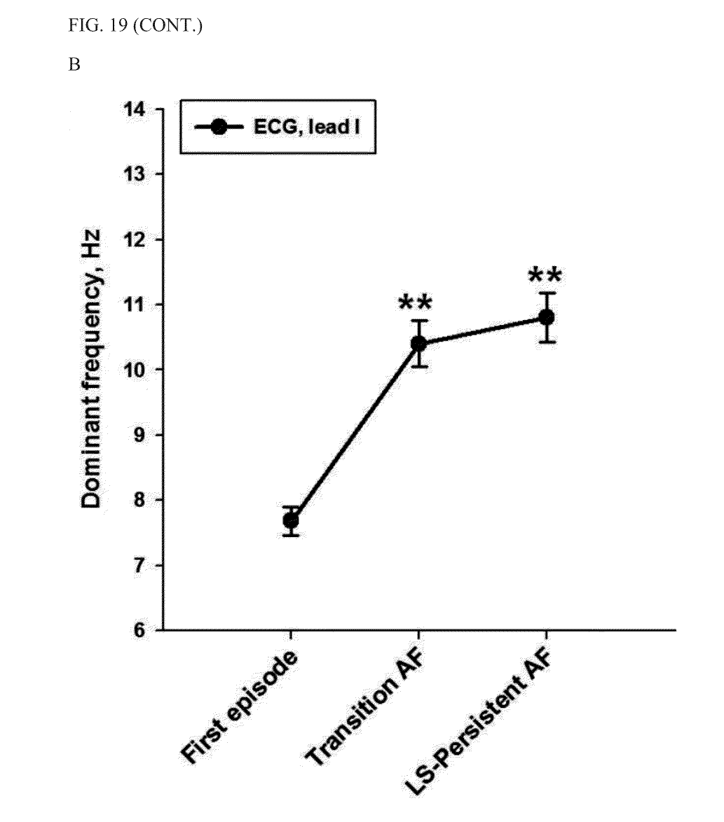

During weekly interrogations, AF occurrence ongoing episodes were assessed. The DF of the first episode recorded from the RA lead was relatively slow at 7.5±0.1 Hz (range 6.5-8.25 Hz). Simultaneous DFs from the surface ECG and ILR after QRST subtraction were 7.7±0.2 Hz (range 6.5 to 9.25 Hz) and 9.0±0.1 Hz (range 8.9-9.4 Hz), respectively. At the outset there was a significant DF difference between RA and LA (p<0.001, FIG. 19). Thereafter, DF increased progressively in both atria. At both transition and LS-PAF, DFs recorded on the RA, surface ECG and LA were higher than during the first episode (p<0.001). However, in the 7 LS-PAF sheep, the last DFs recorded after 1 year of AF were not significantly different from prior corresponding values at transition. Thus, the major increase in DF occurred during paroxysmal AF and not during self-sustained LS-PAF. Additionally, while a significant LA-to-RA frequency gradient was present during the first episode, this gradient diminished at transition (p=0.06) and LS-persistent time points (p=0.1), reflecting remodeling of refractory periods in both atria. In any given animal, once respective maximum DF values were achieved, they remained relatively stable after one year follow up; there was no significant difference between maximum DF at transition and at ˜350 days.

### The Rate of DF Increase Predicts the Onset of Persistent AF

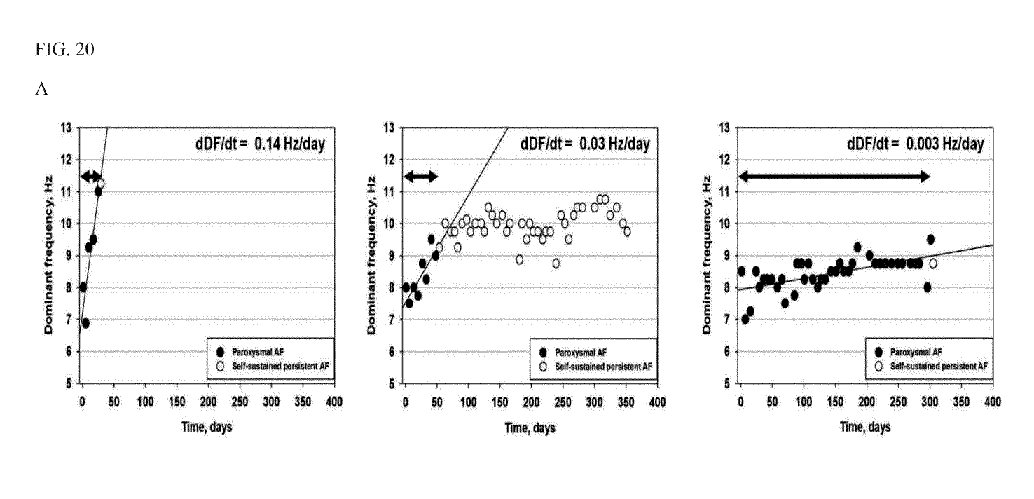

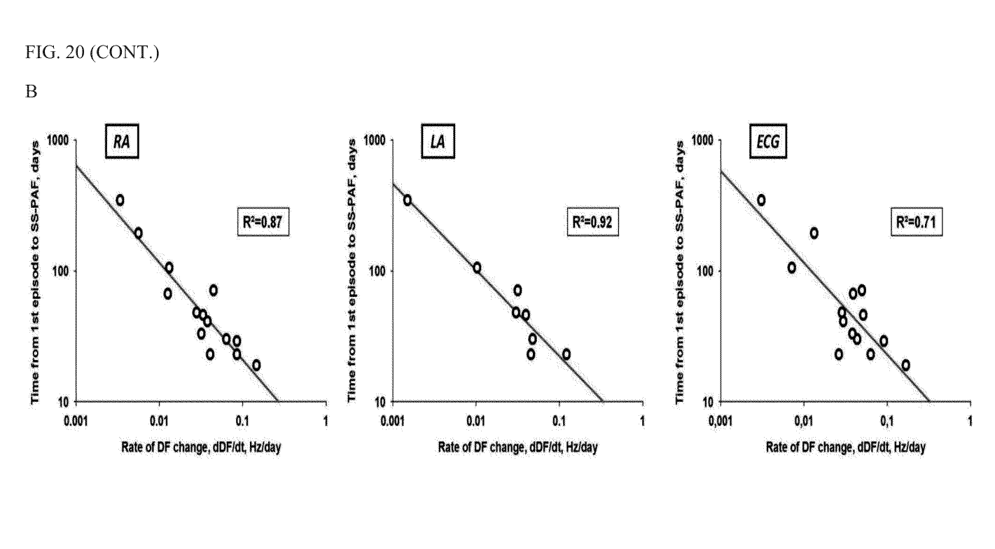

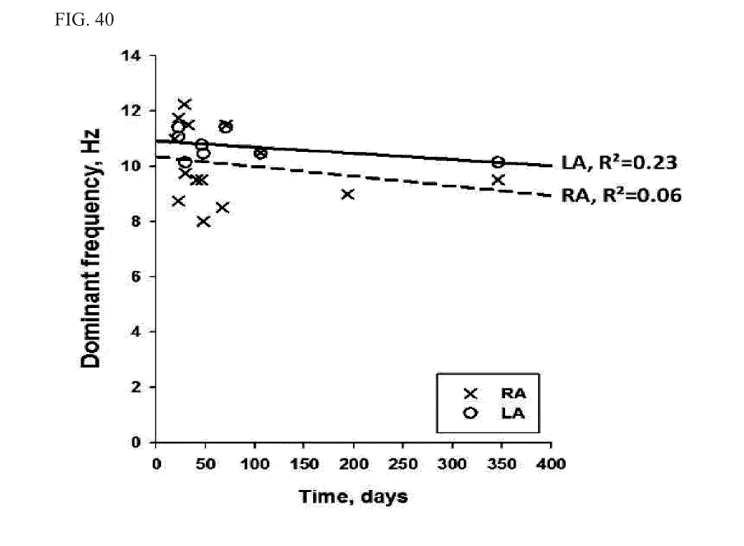

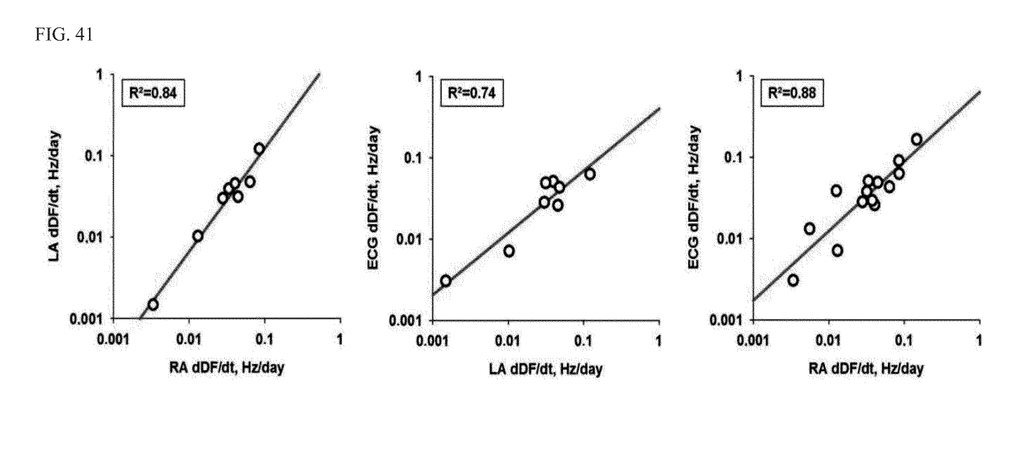

Several parameters were analyzed to determine whether or not the time in paroxysmal AF and transition to self-sustained persistent AF could be predicted. It was first determined if a critical DF was reached before self-sustained persistent AF developed, but the data did not support this hypothesis (FIG. 40). Not only did maximal DF vary among animals, but the rate of DF increase during transition was also highly variable, ranging 0.003 to 0.15 Hz/day in the RA and 0.001 to 0.12 Hz/day in the LA. However, sheep that developed self-sustained persistent AF early, also had a steep slope of DF increase with time (dDF/dt), regardless of DF during the first episode, whereas those with a delayed onset of persistent AF had a shallower DF slope (FIG. 20A). Thus it was hypothesized that dDF/dt could predict when AF became persistent in each animal. Indeed, a strong nonlinear relationship was found between time to persistent AF onset and dDF/dt regardless of whether DF was determined in the RA, LA or surface ECG (R²=0.87, 0.92 and 0.71, respectively, FIG. 20B). The faster the DF increase, the quicker the animal developed self-sustained persistent AF. Furthermore, non-invasive measurement of dDF/dt (surface ECG lead I) correlated strongly with RA and LA dDF/dt (FIG. 41).

### Electrical Remodeling

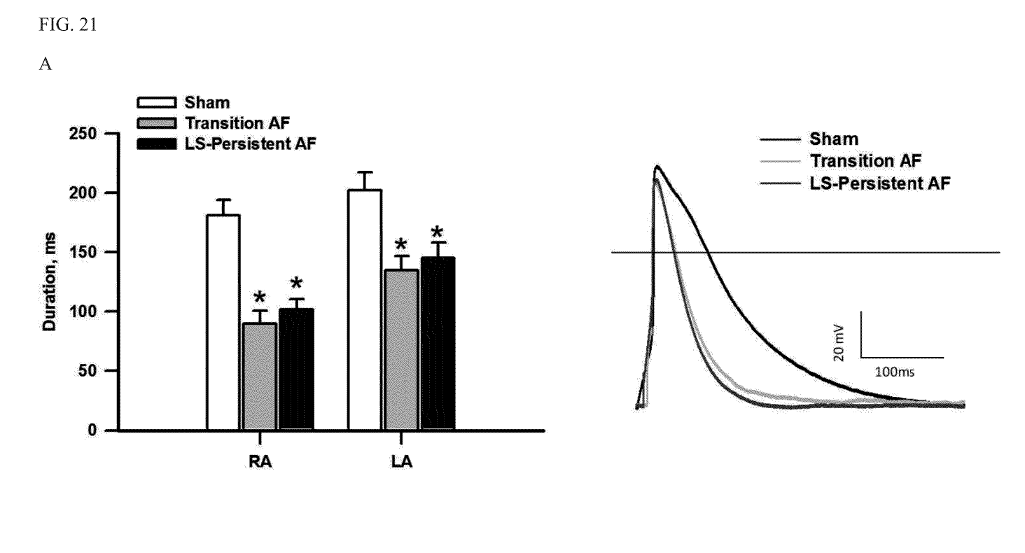

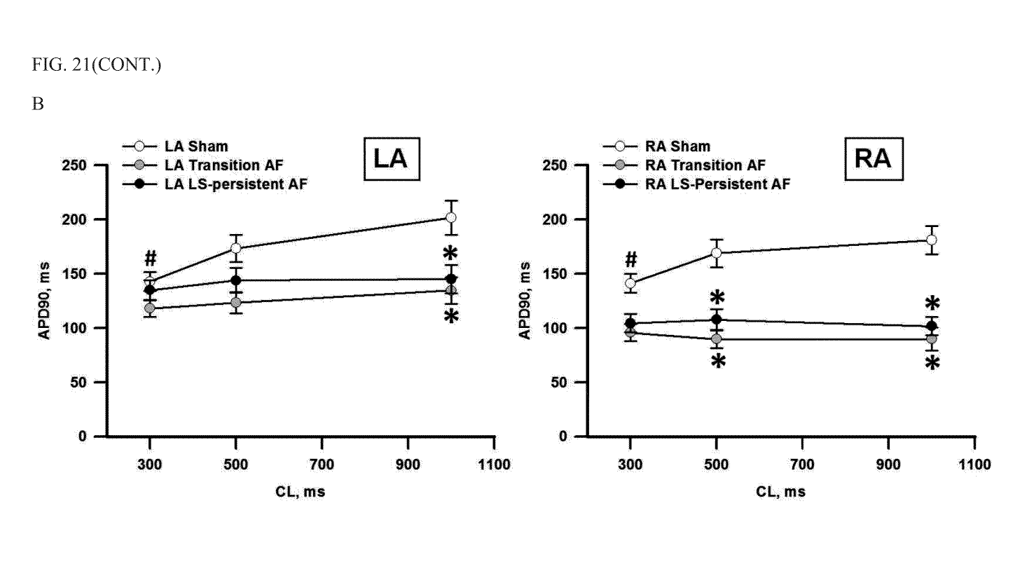

Patch-clamp experiments to determine whether the gradual DF increase during transition reflected development of remodeling at the cellular level were performed. Action potential duration at 90 percent repolarization (APD90) was significantly reduced in both RA and LA at transition and LS-PAF groups (FIG. 21). Sheep from both groups tended to have more hyperpolarized resting membrane potentials than sham (p=NS) for RA (−69.8±2.8 mV, −60.2±3.4 mV and −57.6±4.6 mV, respectively) and LA myocytes (−72.1±4.1 mV, —66.6±3.6 mV and −63.5±2.3 mV, respectively). Action potential (AP) upstroke velocity (dV/dtmax) also tended to be lower in myocytes from AF animals, while AP amplitudes did not change significantly. Myocytes from animals in AF also showed a loss of rate-adaptation of APD (FIG. 21B). Shortest pacing cycle length before AP alternans or failure to capture was significantly longer in sham than transition and LS-PAF groups, as a consequence of APD and ERP shortening in both RA (345.7±37.5 ms, 165.7±62.6 ms and 203.3±26.5 ms, respectively, p<0.05 vs. sham) and LA (358.3±31.2 ms, 218.1±27.5 ms and 249.4±17.7 ms, respectively, p<0.05 vs. sham).

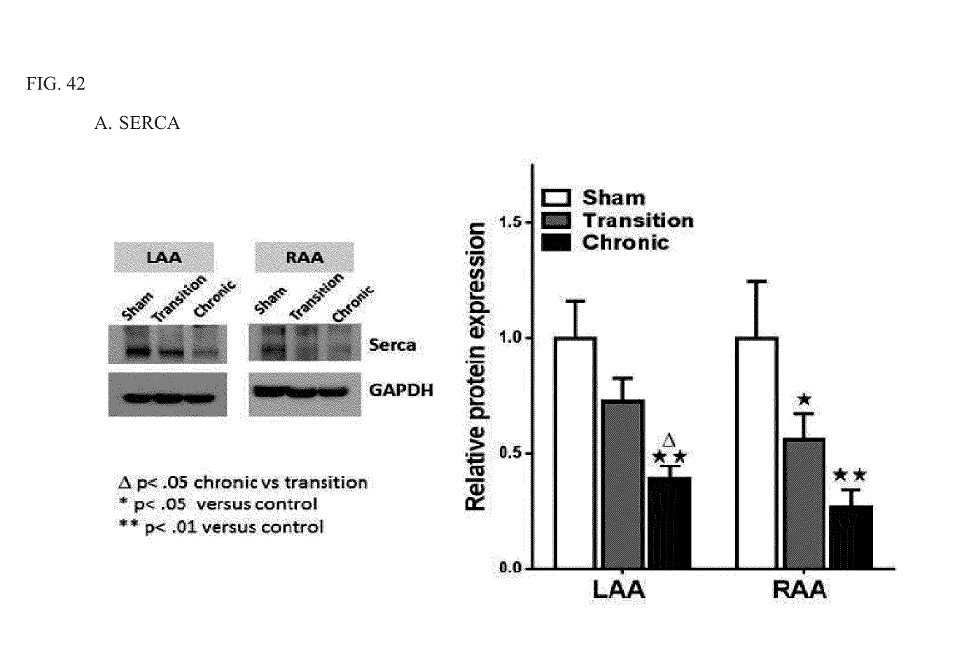

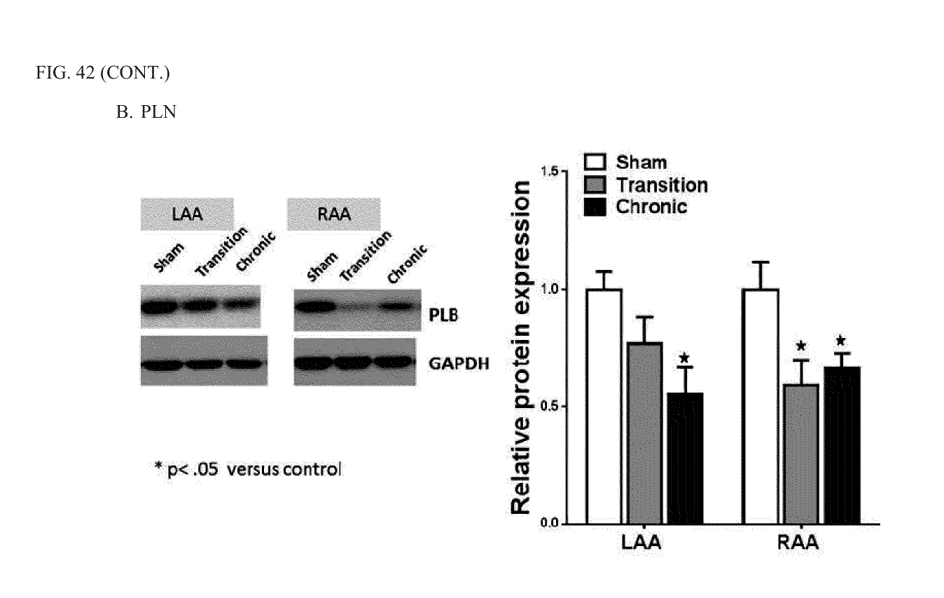

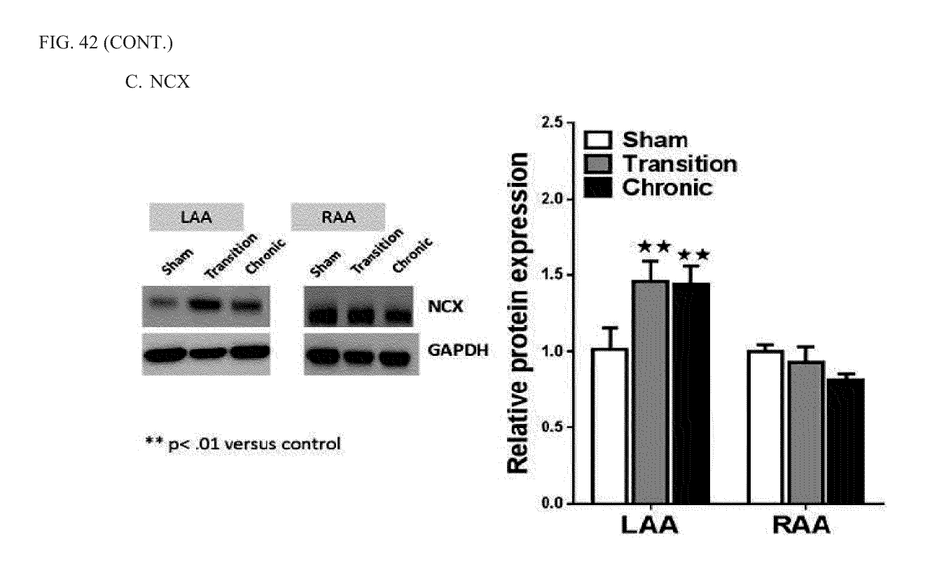

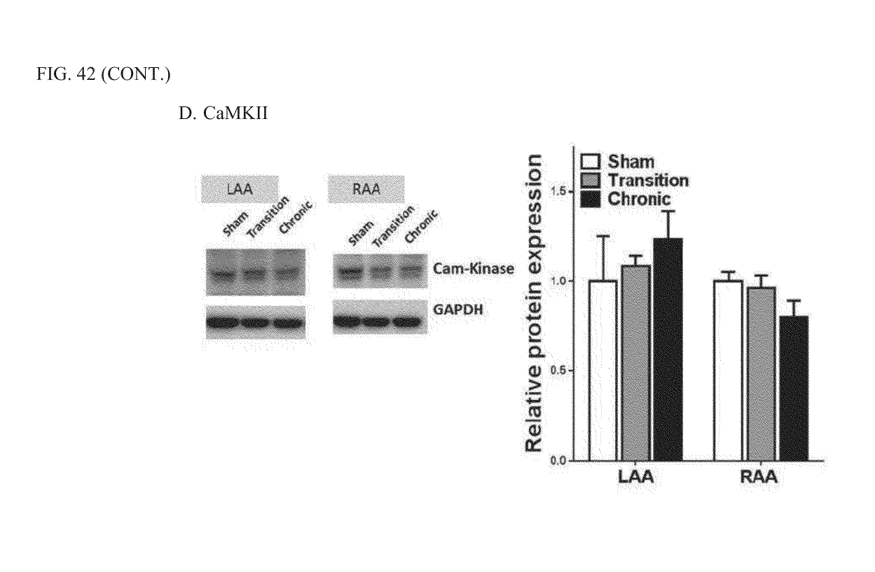

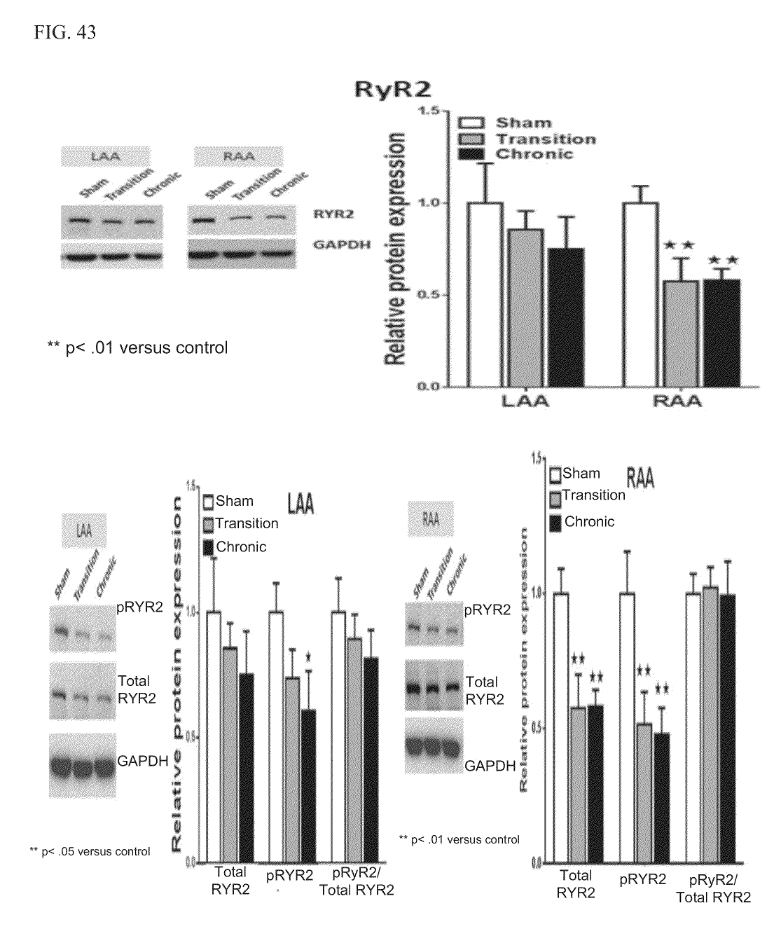

Next, Western blot analyses were conducted in the three groups on animals to test whether remodeling was related to altered intracellular calcium dysfunction. While the Na+—Ca2+ exchanger was increased in the LA Ca2+ leak or delayed afterdepolarizations (FIGS. 42 and 43) (Voigt et al., Circulation. 2012; 125:2059-2070).

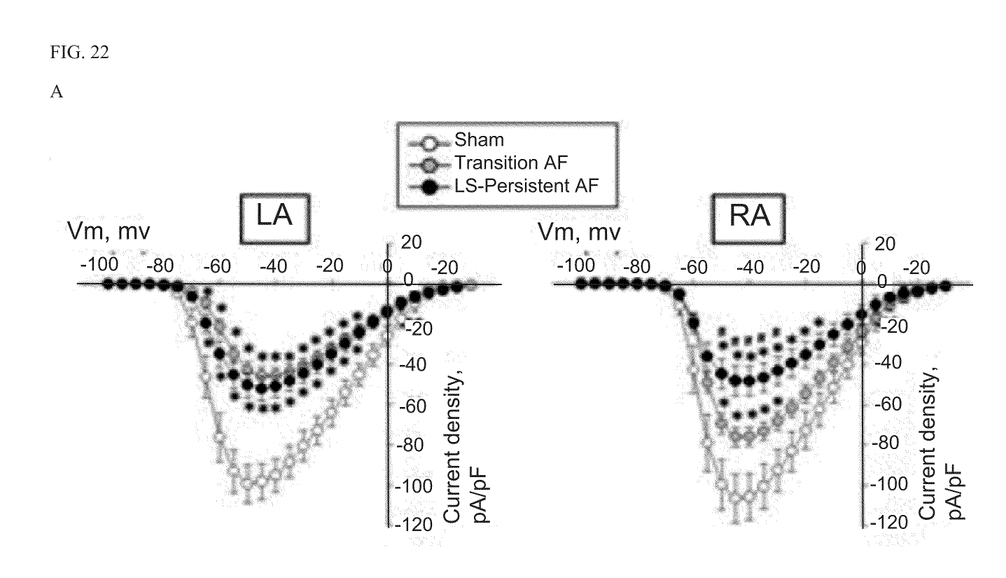

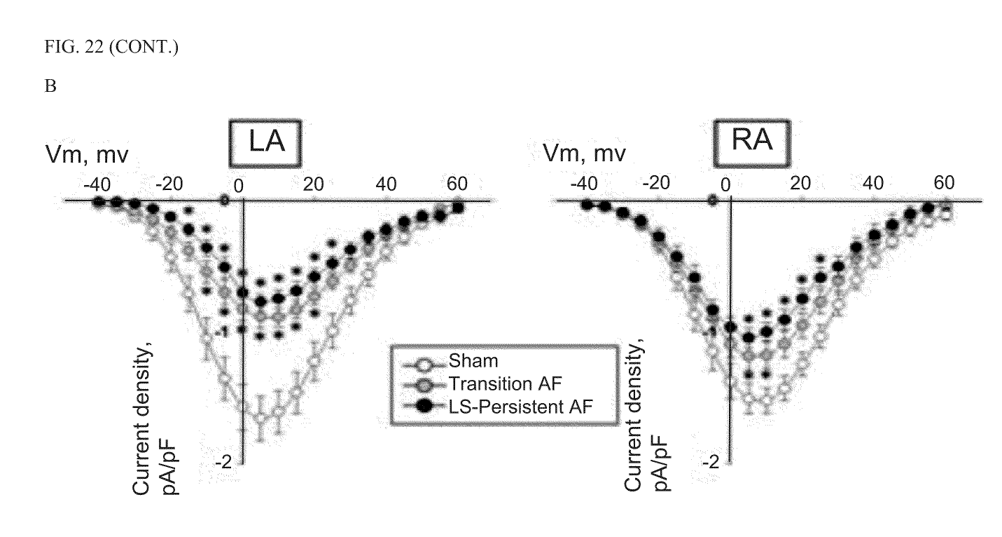

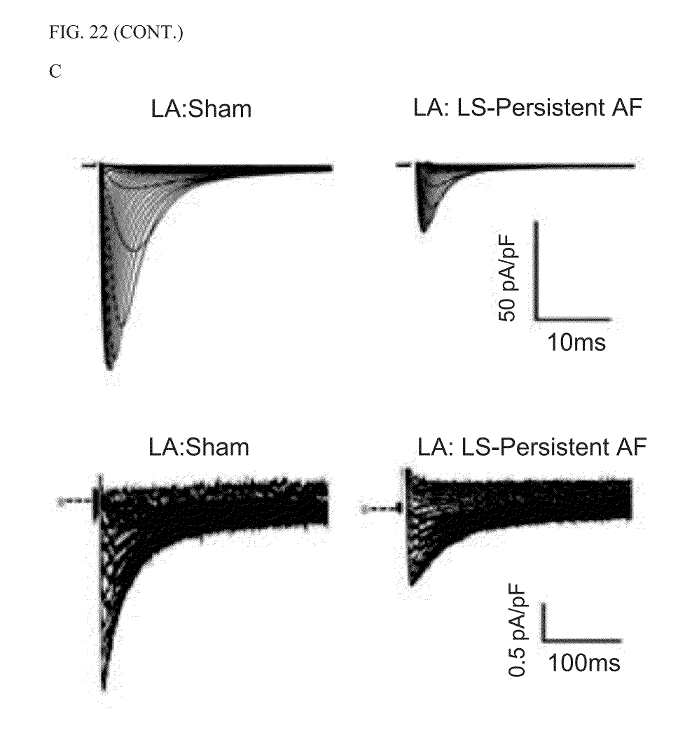

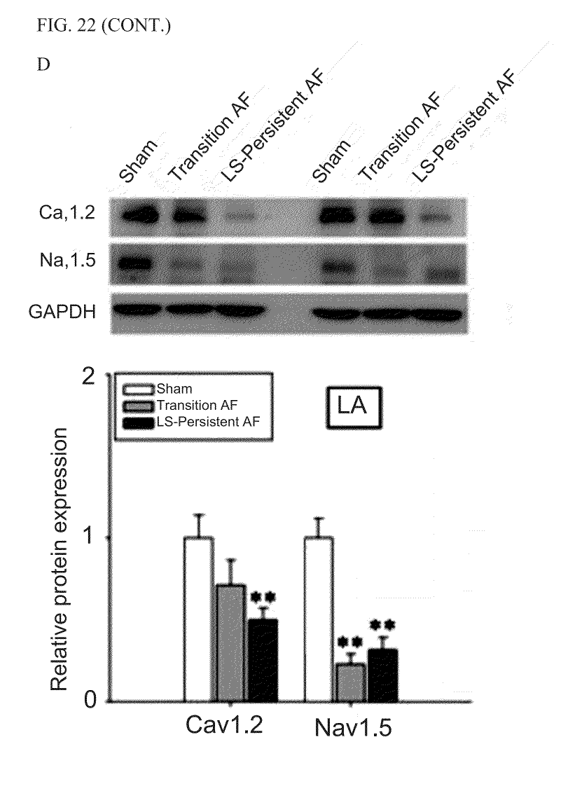

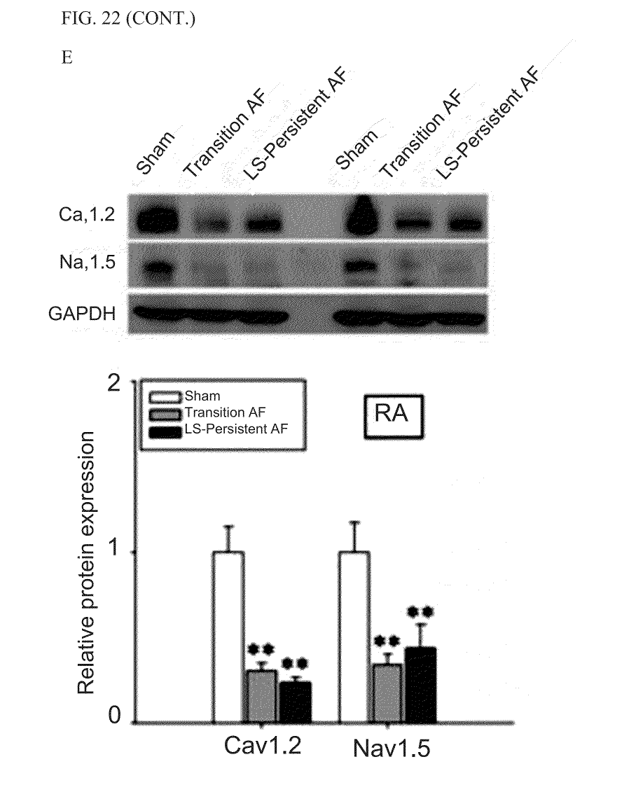

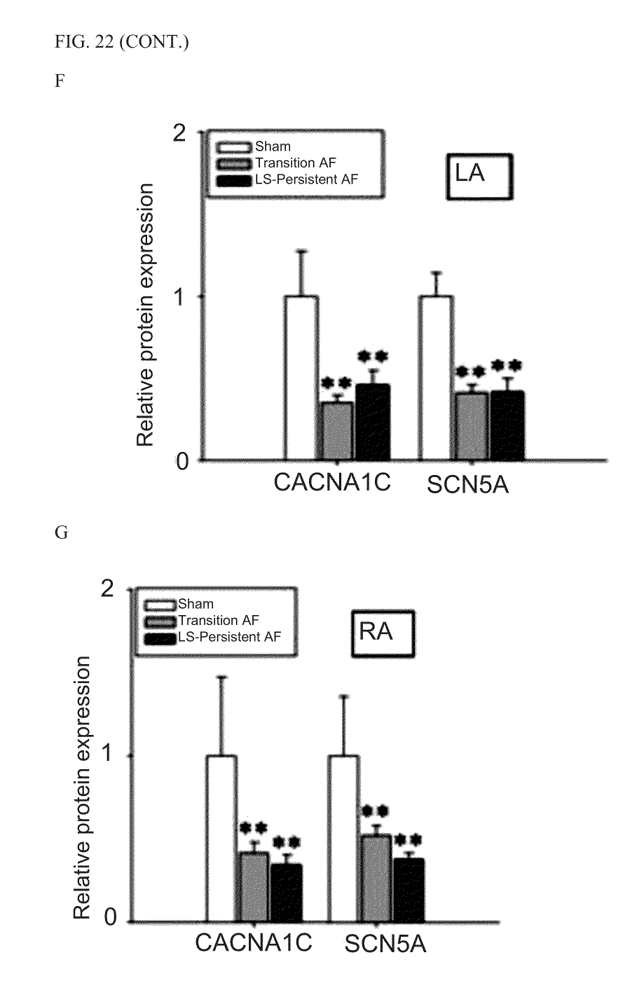

Alterations in sarcolemmal ion channels that contribute to AF-induced changes in APD and refractoriness were investigated. Peak inward sodium current (INa) was significantly reduced at the transition time-point by about 50% in LA myocytes compared to sham (FIG. 22A) and about 30% in RA myocytes. For LS-PAF, peak INa was decreased in both LA and RA myocytes (p<0.001 vs. sham). Similarly, peak L-type calcium current (ICaL) was reduced in LA and RA at transition and LS-PAF (p<0.05, FIG. 22B). Changes in INa and ICaL resulted from concomitant decreases in expression of Nav1.5 and Cav1.2 proteins and SCN5A and remodeling appendage, both total RyR2 and phosphorylated RyR2 proteins were decreased in the AF group, but the ratio of phosphorylated RyR2 to total RyR2 phosphorylation was unaffected. Accordingly, the transition from paroxysmal to persistent AF did not depend on CACNA1C mRNA levels (FIG. 22D-G; see Table 4 for primers used in RT-PCR).

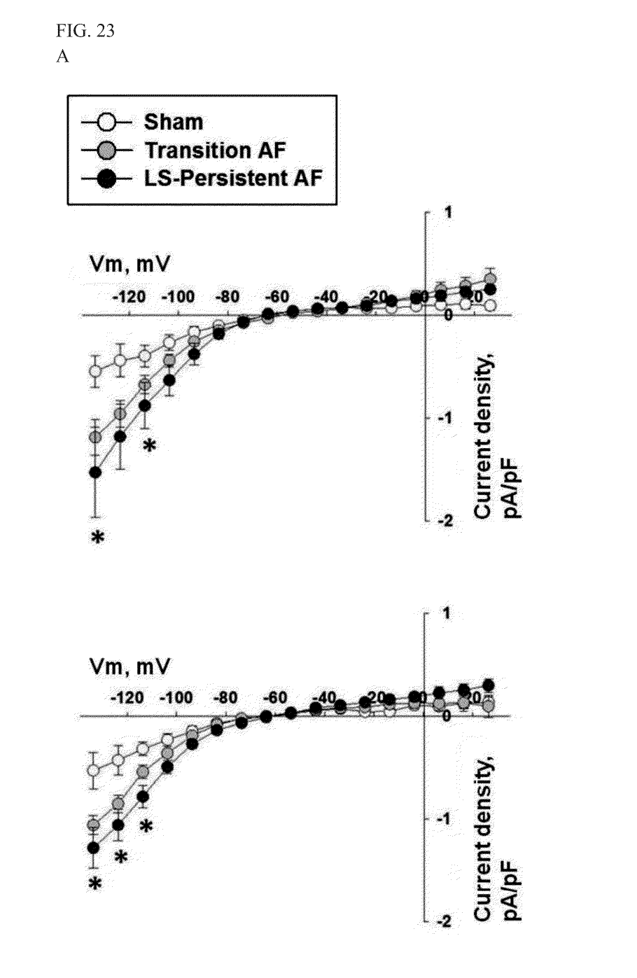

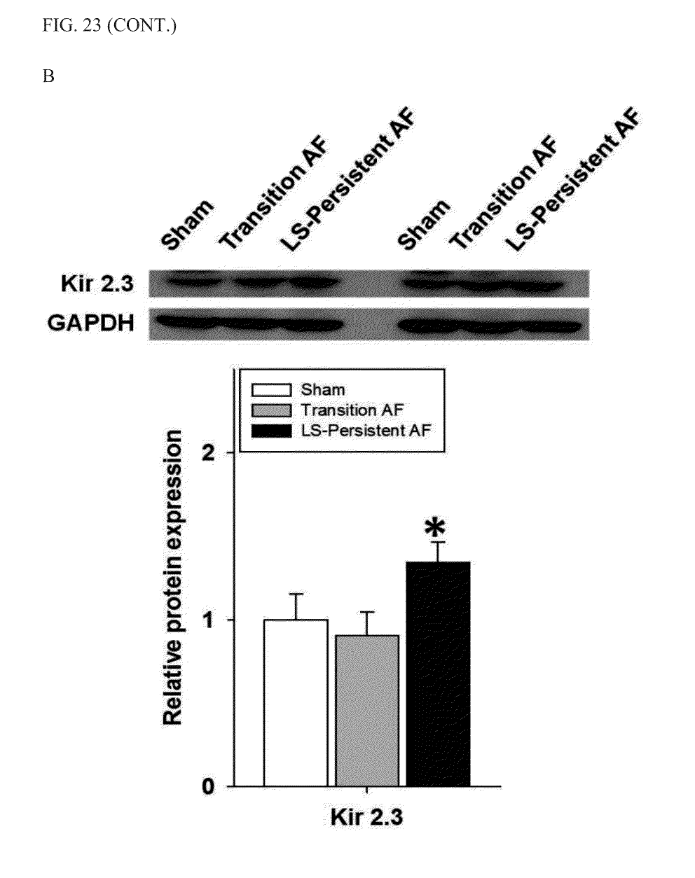

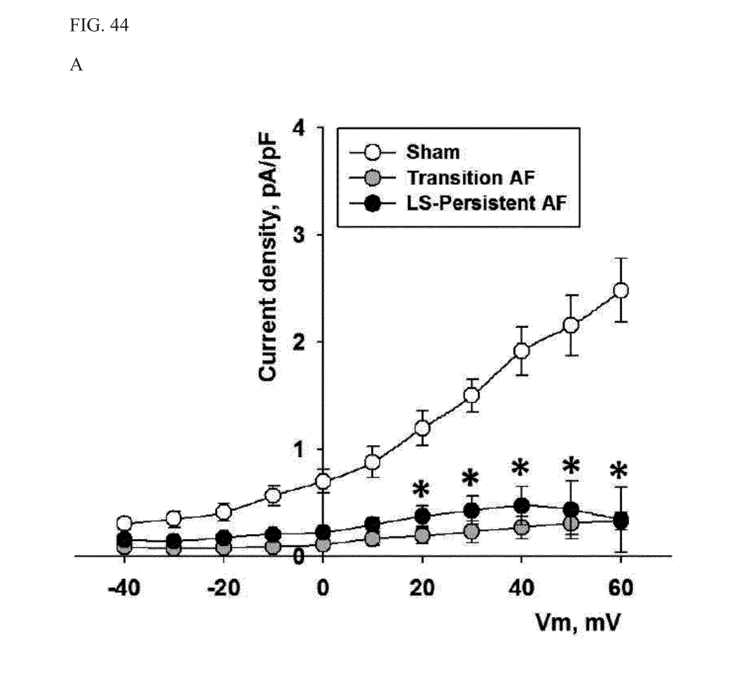

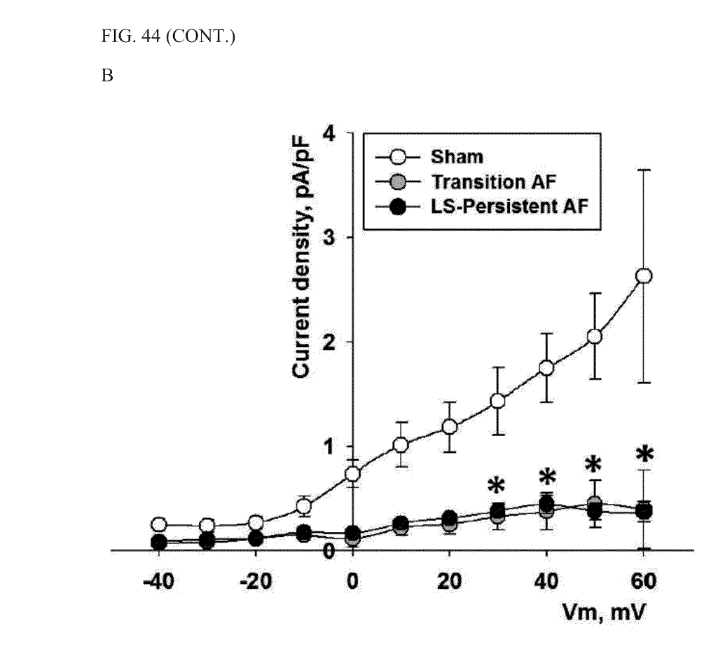

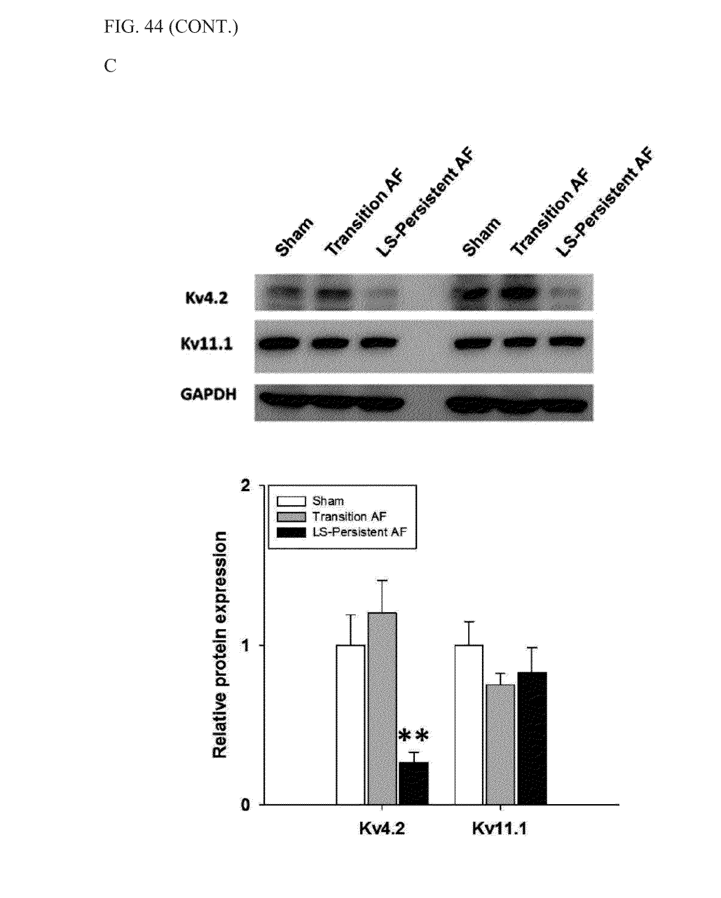

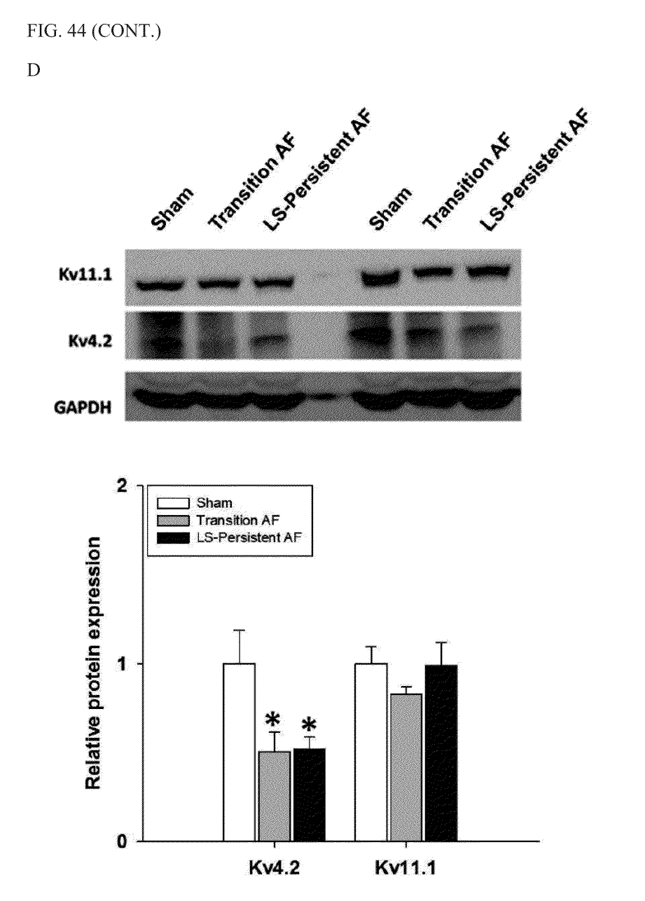

In contrast to INa and ICaL, the density of the inward rectifier potassium current (IK1) increased 2- to 3-fold at negative membrane voltages during the transition in both atria, and continued to increase for LS-PAF (p<0.05 vs. sham, FIG. 23A). Since sheep atria predominantly express Kir2.3 channels (Dhamoon et al., Circ Res. 2004; 94:1332-1339), Kir2.3 expression, which was increased in LS-PAF animals (FIG. 23B), was measured. There was no Kir2.3 increase in transition despite the larger current density compared to sham. The transient outward K+ current (Ito) decreased by about 85% by transition (FIG. 44) and remained low in LS-PAF (p<0.001; FIG. 44). For LSPAF animals, Ito reduction is explained by decreased Kv4.2 expression. However, reduced protein was not evidenced in the LA in transition animals (Tessier et al., Circ Res. 1999; 85:810-819). Lastly, Kv11.1 protein expression remained unchanged (FIG. 44C-D).

### Ionic Current Changes

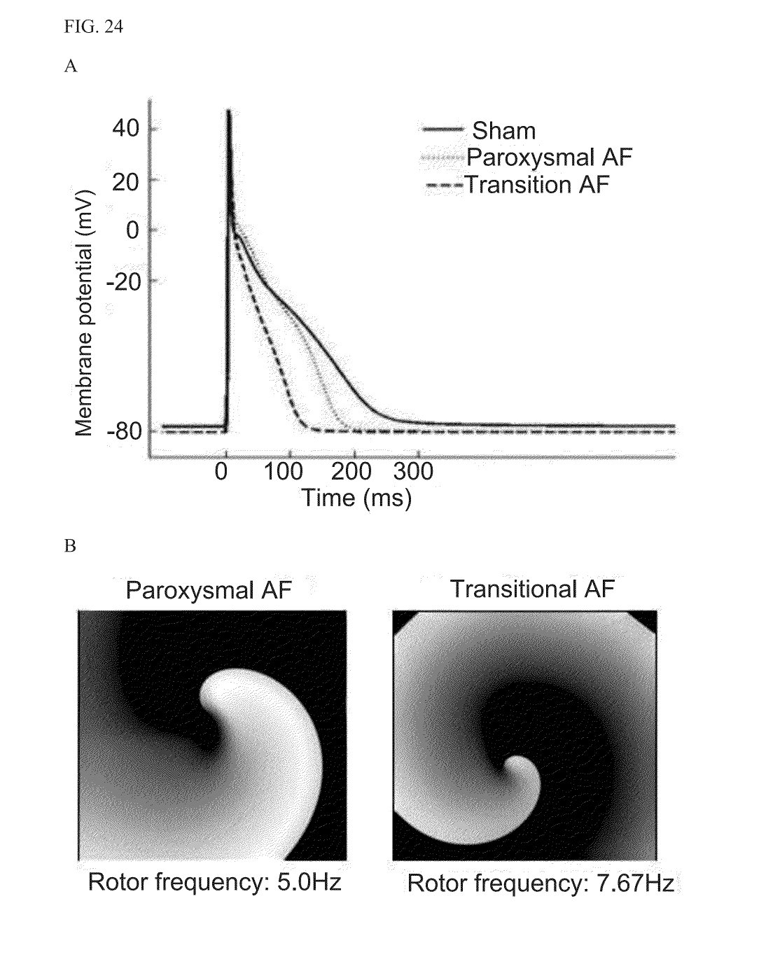

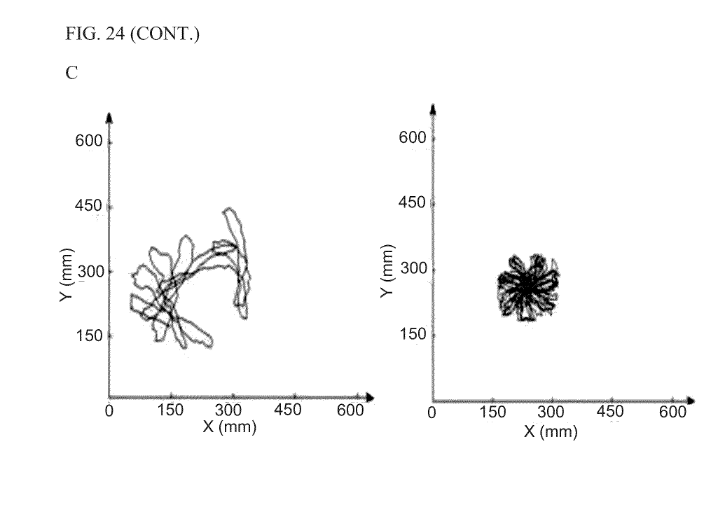

APs were generated for control, paroxysmal, and transition AF conditions using the Grandi-Pandit human atrial AP model (FIG. 24A, Table 5). The ionic changes for the transition AF were based on experimental patch clamp recordings. To represent paroxysmal AF, the ionic changes made in transition AF were retained and the magnitude of ICaL was reduced by only 30% (Table 5), such that the simulated APD90 was shortened significantly by 17% in paroxysmal AF, compared to 51% in transition AF (Table 6).

A 2D sheet model of reentry was used to investigate whether AP differences between paroxysmal and transition AF simulations were responsible for the progressive DF increase demonstrated in vivo. Sustained functional reentry (rotor) dynamics showed differential properties. The rotor in paroxysmal AF (FIG. 24B, left) was short lived, and exhibited low rotation frequency (5.0 Hz) and considerable meandering (FIG. 24C, left), eventually self-terminating upon collision with boundary edges. In contrast, in the transition AF model, the rotor was stable and persisted throughout the length of the simulation (FIG. 24B, right) with significantly less rotor meander (FIG. 24C, right) and higher DF (7.67 Hz) compared to the transition case. When reduction in INa density was not incorporated, the DF increased only slightly to 8.67 Hz, but the rotor was unstable and eventually stopped.

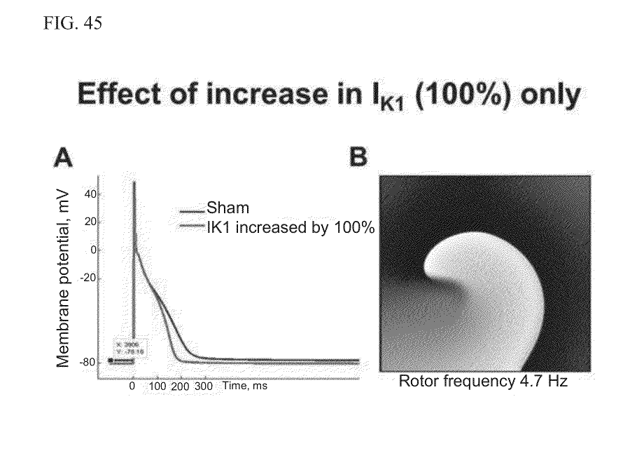

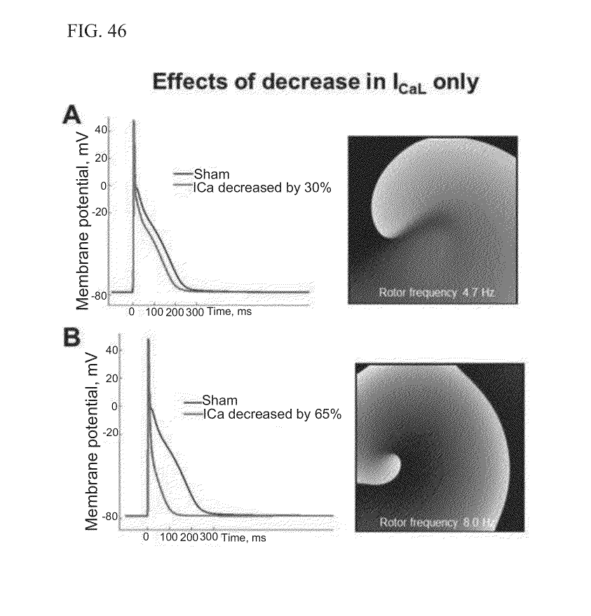

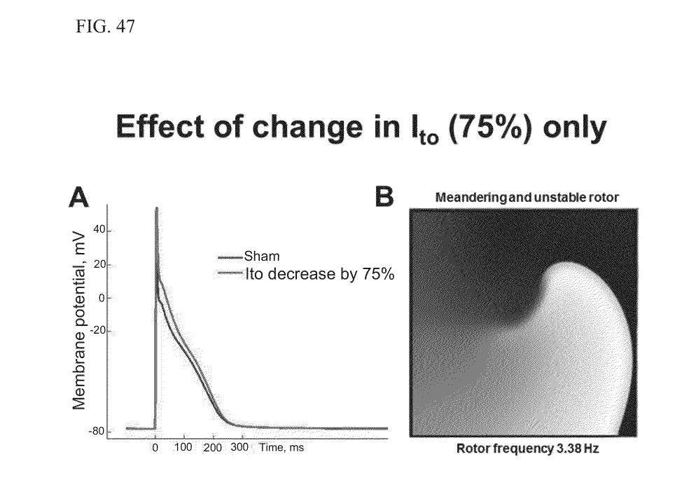

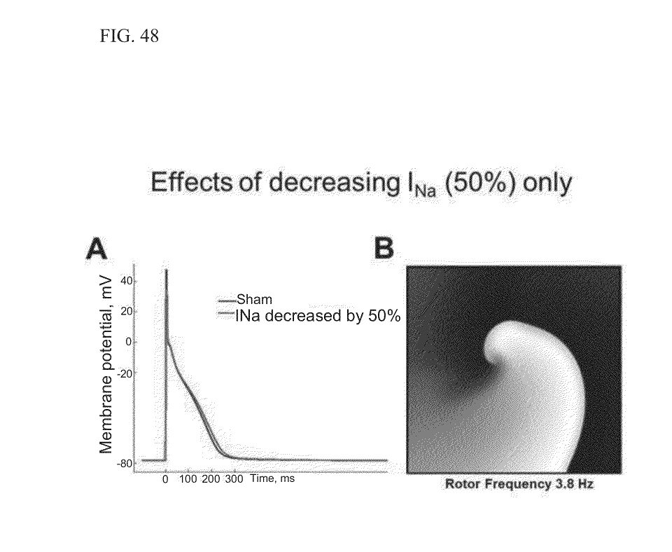

The roles of individual ionic changes in a subset of simulations were investigated. Rotors were simulated in 2D sheets, when individual ionic currents were changed, compared to controls. The simulation results confirmed that changes in IK1 and ICaL are key determinants of rotor acceleration in paroxysmal and transition AF (FIGS. 45-48).

Fast Versus Slow Transition

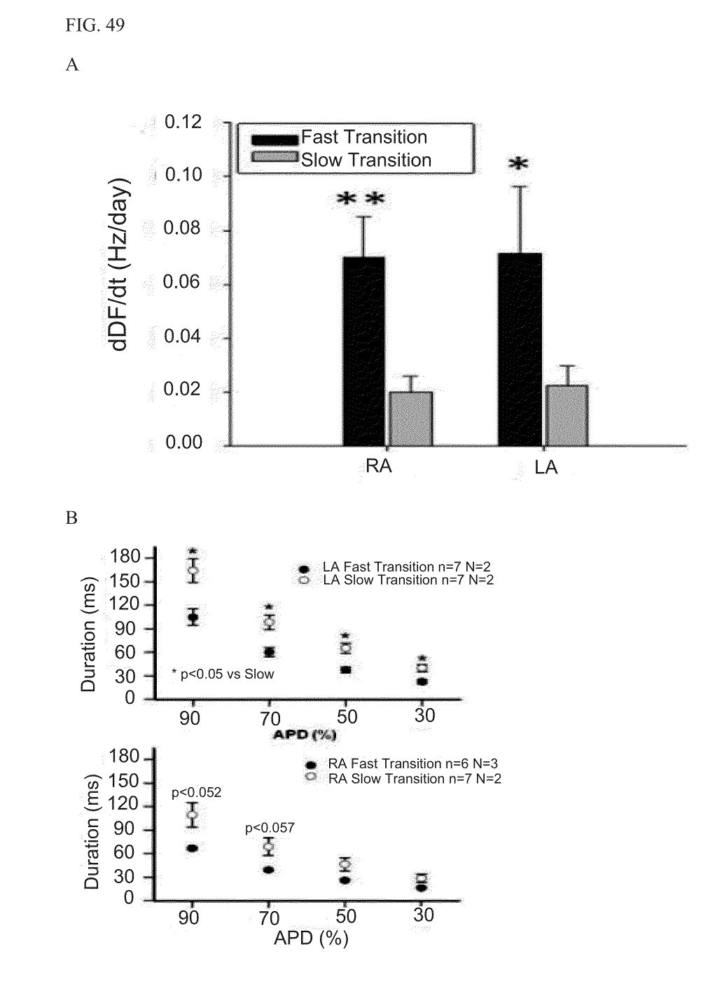

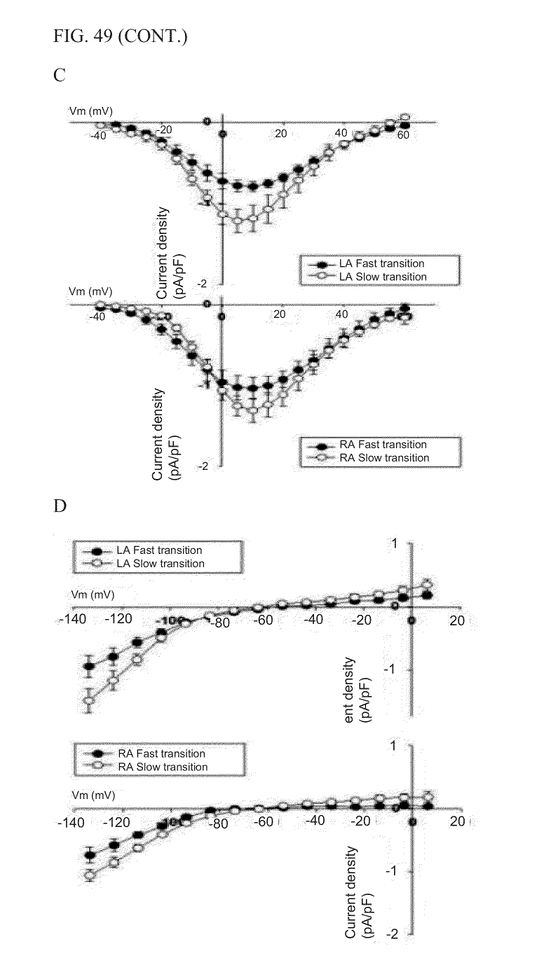

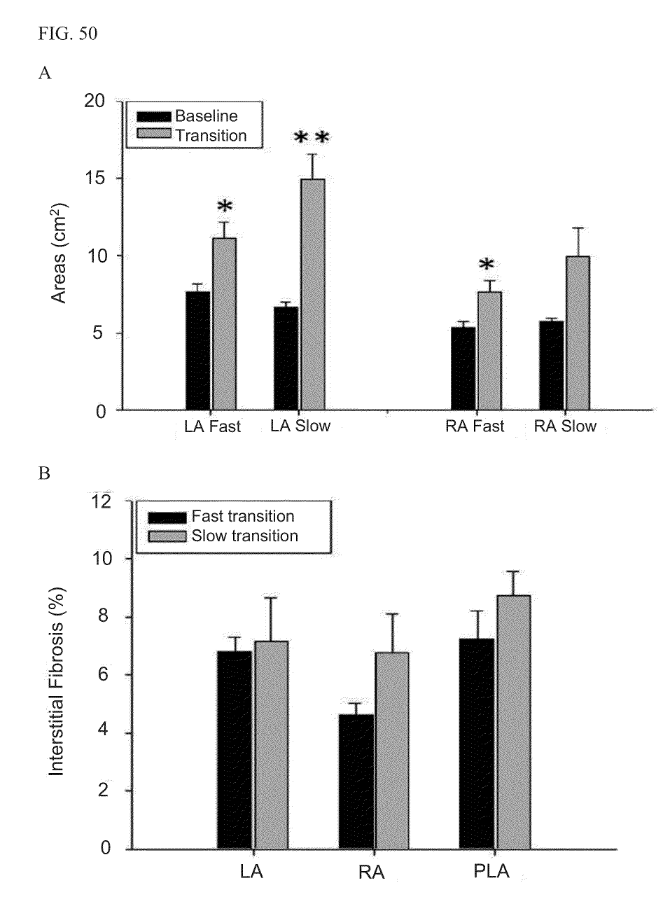

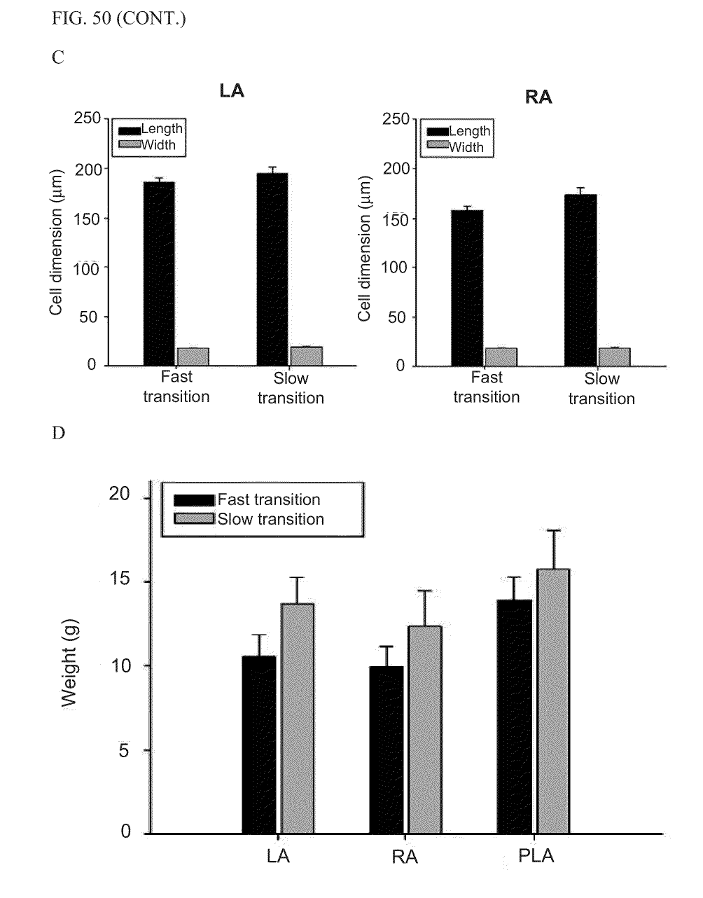

To search for determinants of the rate of AF progression, slow and fast progressing animals were sacrificed at transition depending on the median time to progression (<45 days: 4 animals; >45 days: 3 animals). The major factor contributing to the larger dDF/dt in the fast transition animals was greater APD shortening secondary to ICaL reduction (FIGS. 49 and 50). The slow transition animals required an additional IK1 increase and greater structural remodeling.

Gal-2 Inhibition Trial

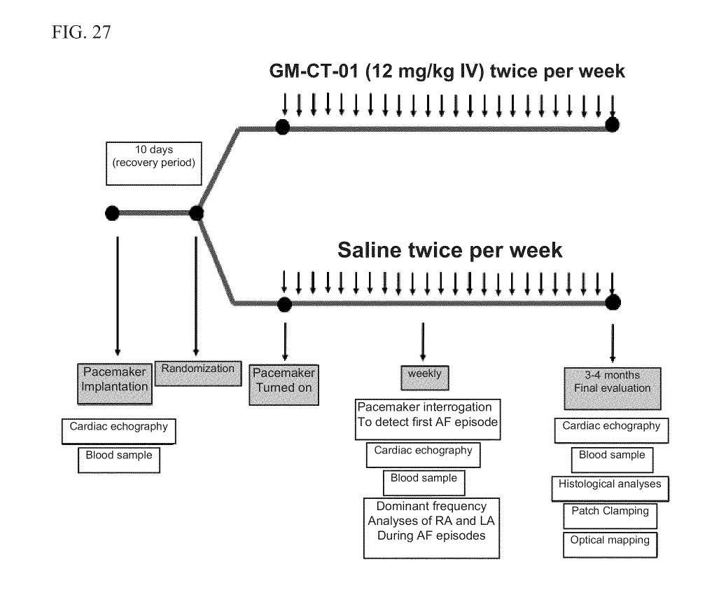

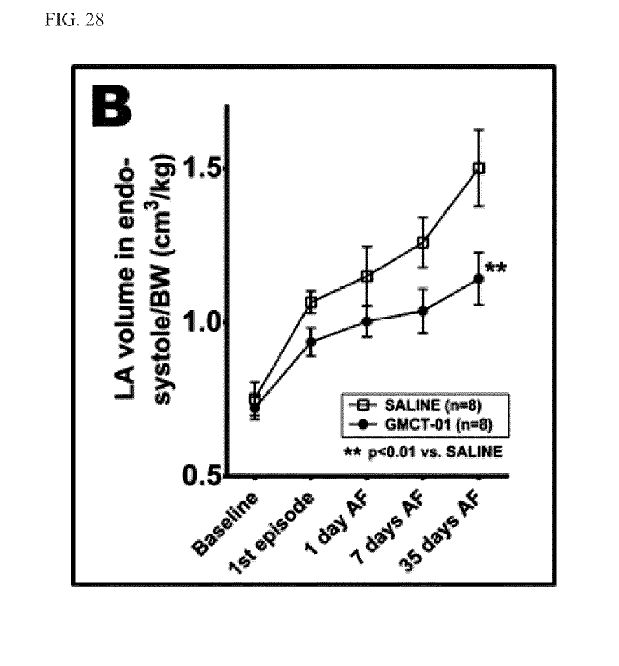

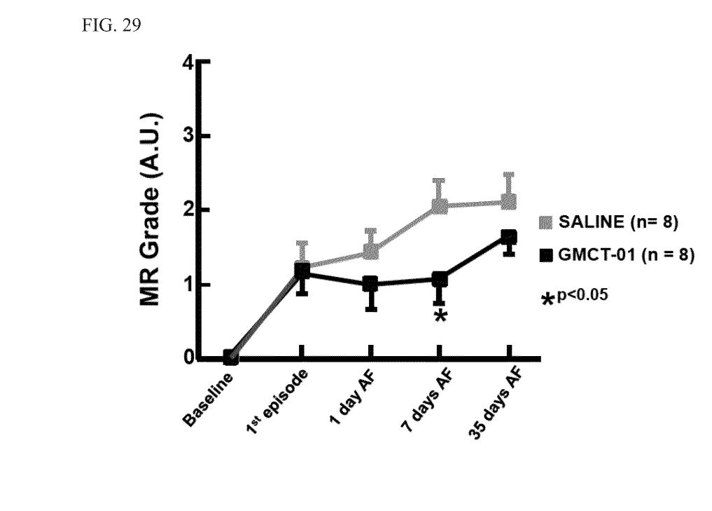

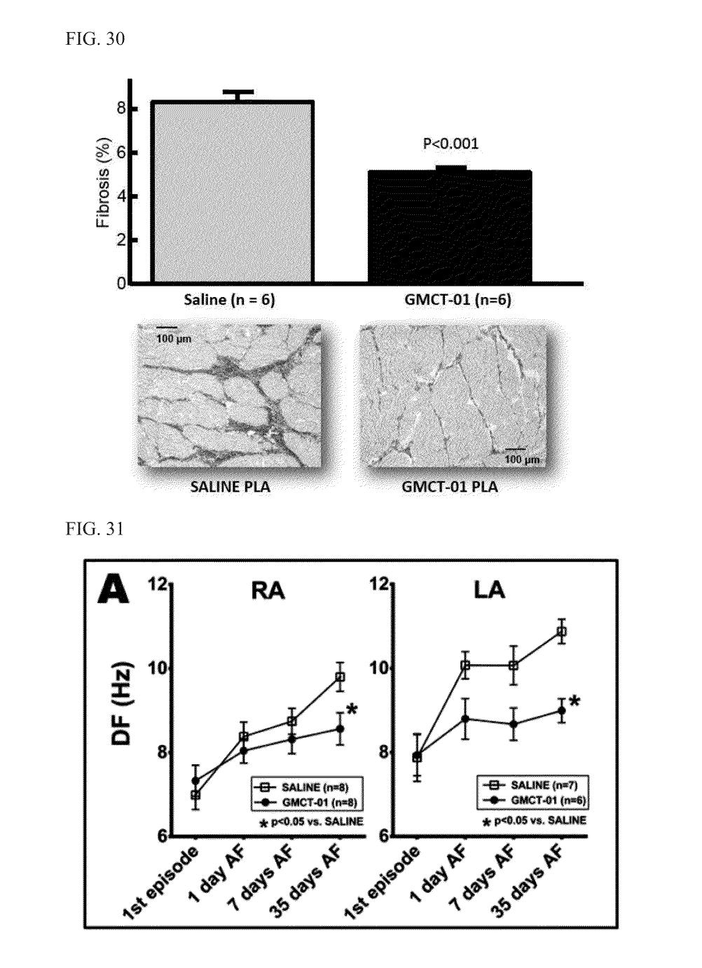

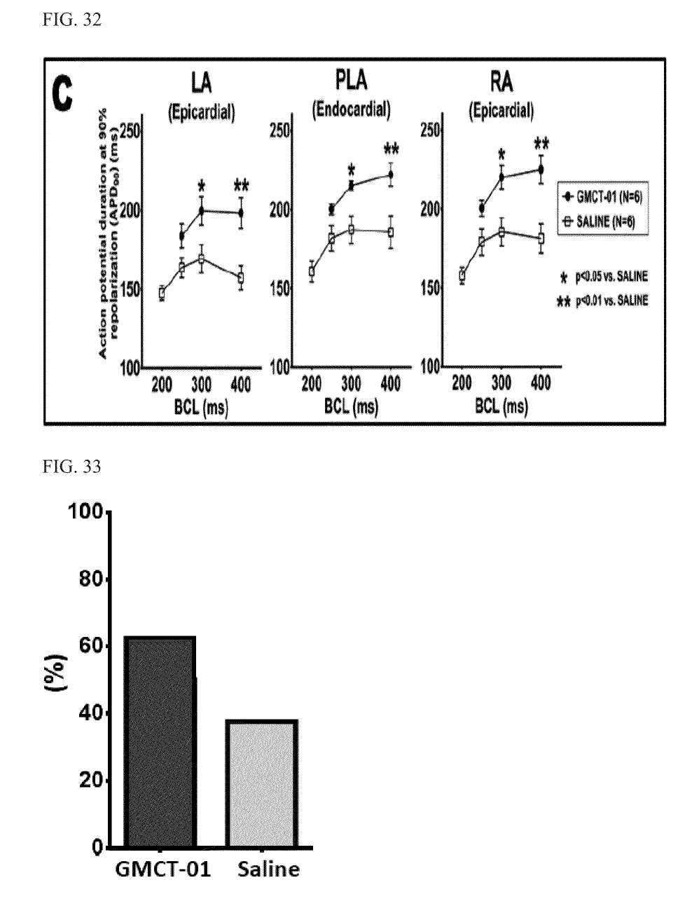

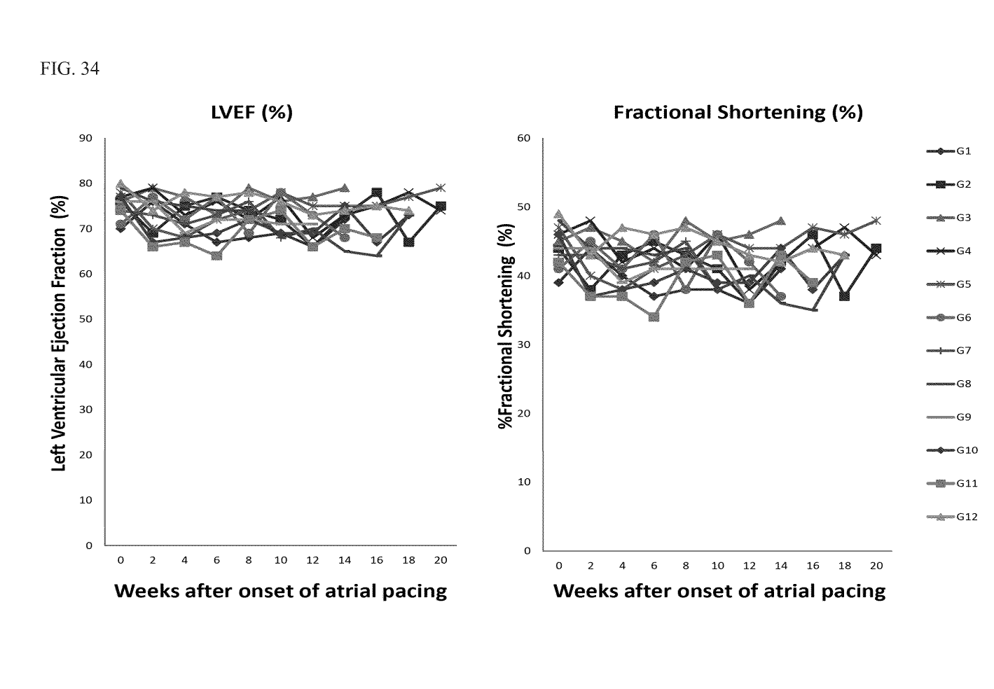

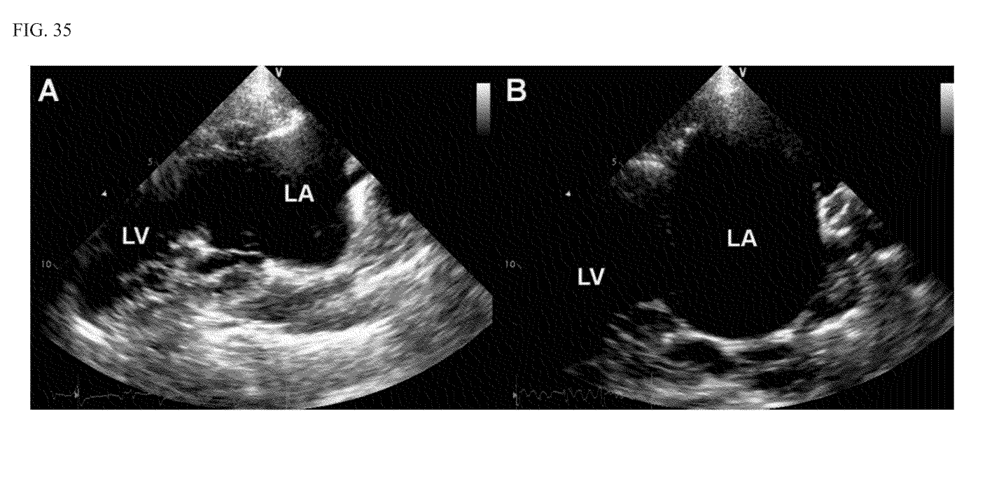

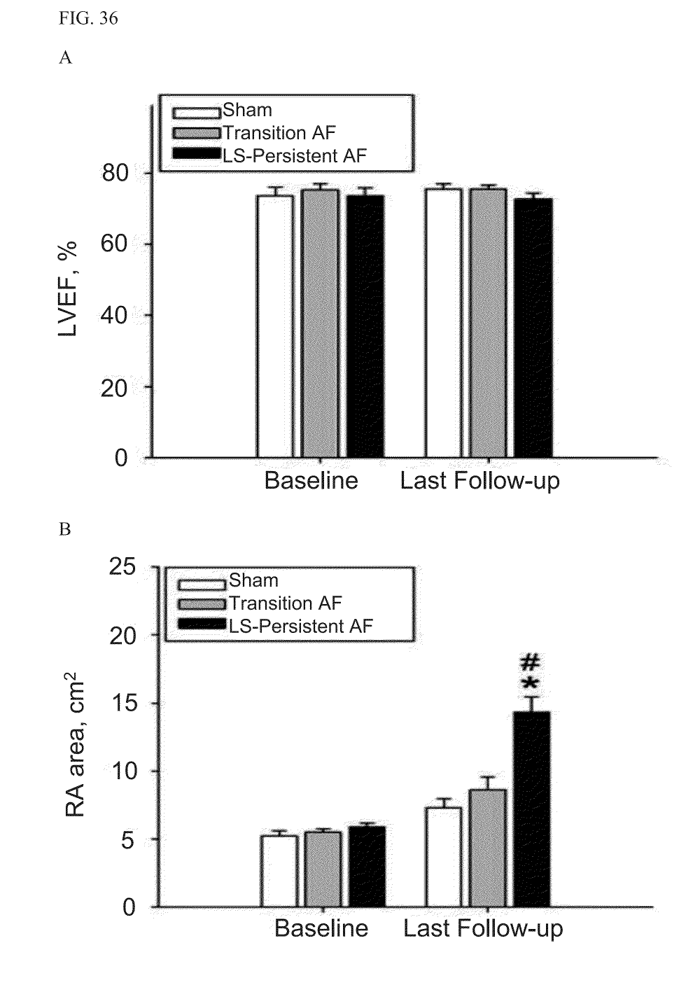

A trial of Gal-3 inhibition in the sheep model was conducted. FIG. 27 shows a protocol for a Gal-3 inhibitor trial. Results are shown in FIGS. 28-34 and show that Gal-3 inhibition reduces both AF-induced structural and electrical remodeling in the sheep model of persistent AF. For example, FIG. 28 shows that Gal-3 inhibition lessens AF-induced atrial dilatation. FIG. 29 shows that Gal-3 inhibition reduces mitral regurgitation (MR). FIG. 30 shows that Gal-3 inhibition reduces Fibrosis in the PLA. FIG. 31 shows that Gal-3 inhibition prevents the sustained AF increase in dominant frequency as measured in both RA and LA. FIG. 32 shows that Gal-3 inhibition prevents the sustained AF-induced shortening of action potential duration in both RA and LA. FIG. 33 shows that Gal-3 Inhibition increases the percentage of spontaneous terminations of persistent AF during treatment. FIG. 34 shows that Gal-3 inhibition does not alter left ventricular function.

TABLE 1

| | | Transition | | |
|---|---|---|---|---|
| | Sham | AF | LS-PAF | p |
| LVEF (%) | | | | |
| Baseline | 73.7 ± 2.4 | 75.3 ± 1.7 | 73.7 ± 2.2 | 0.82 |
| Last follow-up | 75.5 ± 1.4 | 75.6 ± 1.0 | 72.7 ± 1.7 | 0.30 |
| LA area (cm²) | | | | |
| Baseline | 7.6 ± 0.3 | 7.2 ± 0.4 | 7.7 ± 0.2 | 0.56 |
| Last follow-up | 10.0 ± 0.8 | 12.8 ± 1.1 | 20.9 ± 2.1* | 0.004 |
| RA area (cm²) | | | | |
| Baseline | 5.2 ± 0.4 | 5.5 ± 0.2 | 5.9 ± 0.3 | 0.30 |
| Last follow-up | 7.3 ± 0.7 | 8.6 ± 1.0 | 14.3 ± 1.0*† | 0.006 |
| Mitral | | | | |
| regurgitation, /4 | | | | |
| Baseline | 0.0 ± 0.0 | 0.1 ± 0.1 | 0.0 ± 0.0 | 0.19 |
| Last follow-up | 0.1 ± 0.1 | 0.8 ± 0.3 | 1.2 ± 0.2* | 0.03 |

TABLE 2

| Tissue | | | | |
|---|---|---|---|---|
| Region | Sham | Transition | LA = PAF | |
| LA | 6.5 ± 0.6 g | 10.6 ± 1.3 g (NS) | 15.4 ± 2.1 g | (p < 0.02) |
| RA | 7.4 ± 0.8 g | 9.9 ± 1.2 g (NS) | 17.3 ± 2.8 g | (p < 0.04) |
| PLA | 9.8 ± 1.7 g | 13.9 ± 1.3 g (NS) | 20.1 ± 2.9 g | (p < 0.04) |

TABLE 3

| | Sham | Transition AF | LS-PAF | p |
|---|---|---|---|---|
| LA cell size | | | | |
| Length, μm | 153.3 ± 4.1 | 188 ± 4.0* | 186.1 ± 4.1* | <0.001 |
| Width, μm | 16.2 ± 0.4 | 18.2 ± 0.4† | 19.0 ± 0.4* | <0.001 |
| RA cell size | | | | |
| Length, μm | 155.4 ± 3.7 | 161.9 ± 4.2 | 168.8 ± 3.8 | 0.07 |
| Width, μm | 17.0 ± 0.9 | 18.3 ± 0.4 | 21.4 ± 0.5§ | <0.05 |
| Patchy | | | | |
| Fibrosis, % | | | | |
| RA | 5.4 ± 0.4 | 5.4 ± 0.4 | 6.3 ± 0.6 | 0.30 |
| LA | 5.0 ± 0.4 | 5.8 ± 0.8 | 6.2 ± 0.7 | 0.49 |
| PLA | 6.2 ± 0.7 | 6.6 ± 0.8 | 9.3 ± 1.2 | 0.13 |
| Interstitial | | | | |
| Fibrosis, % | | | | |
| RA | 5.1 ± 0.9 | 5.5 ± 0.7 | 6.6 ± 0.6 | 0.34 |
| LA | 5.5 ± 1.2 | 7.0 ± 0.6 | 10.7 ± 1.5§ | 0.018 |
| PLA | 4.1 ± 0.6 | 7.9 ± 0.7 | 14.6 ± 1.4‡ | <0.001 |

TABLE 4

| | | | SEQ | Forward Primer | SEQ | Reverse Primer |
|---|---|---|---|---|---|---|
| Gene | Protein | | ID NO.: | (5′->5′) | ID NO.: | (5′->3′) |
| CACNA1C | Cav1.2 | XM_004007606.1 | 1 | GGAGCGGGTGGAGTATCTCT | 7 | GAGGTAAGCGTTGGGGTGAA |
| SCN5A | Nav1.5 | XM_004018231.1 | 2 | GCAACTTCACGGTGCTCAAC | 8 | TGAGGTAGAGGTCCAGCGAT |
| KCND2 | Kv4.2 | XM_004008268.1 | 3 | GGAAGCTCCACTATCCTCGC | 9 | CGGCGATCCTTGTACTCCTC |
| KCNJ4 | Kir2.3 | XM_004023651.1 | 4 | CTACTTCGCCAACCTGAGCA | 10 | TCATGAGCATGTAGCGCCAG |
| KCNJ3 | Kv4.3 | XM_004002124.1 | 5 | CTCCACCATCAAGAACCACGA | 11 | CGTGTGGACGGGTAGTTCTG |
| KCNJ2 | Kir2.1 | XM_004013146.1 | 6 | CCCTCACGAGCAAAGAGGAA | 12 | GCCTGGTTGTGCAGGTCTAT |

TABLE 5

| Current | Paroxysmal AF | Transition AF |
|---|---|---|
| I_{Na} | −50% | −50% |
| I_{CaL} | −30% | −65% |
| I_{K1} | +100% | +100% |
| Iₜₒ | −75% | −75% |

TABLE 6

| | | Paroxysmal AF | Transition AF |
|---|---|---|---|
| APD | Sham (ms) | (ms) | (ms) |
| APD₃₀ | 5.5 | 15.0 | 8.5 |
| APD₅₀ | 42.0 | 64.5 | 31.0 |
| APD₉₀ | 203.0 | 168.5 | 99.5 |

All publications and patents mentioned in the above specification are herein incorporated by reference. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of treating atrial fibrillation or preventing persistent atrial fibrillation in a subject, comprising: administering an agent that inhibits at least one activity of a galectin polypeptide to said subject, wherein said administering treats or prevents atrial fibrillation in said subject.

2. The method of claim 1, wherein said agent is selected from the group consisting of a small molecule, a carbohydrate, an siRNA, and an antibody.

3. The method of claim 1, wherein said galectin polypeptide is galectin-2 or galectin-3.

4. The method of claim 1, wherein said carbohydrate is pectin or a derivative thereof.

5. The method of claim 4, wherein said pectin is citrus pectin.

6. The method of claim 4, wherein said carbohydrate is formulated as a nutritional supplement or a food additive.

7. The method of claim 1, wherein said subject has been diagnosed with atrial fibrillation.

8. The method of claim 6, wherein said atrial fibrillation is paroxysmal.

9. The method of claim 1, wherein said subject is at risk of persistent atrial fibrillation.

10. The method of claim 1, wherein said subject has had at least one prior incident of atrial fibrillation.

11. The method of claim 10, wherein said administering prevents future incidents of atrial fibrillation in said subject.

12. The method of claim 10, wherein said administering prevents said atrial fibrillation from becoming persistent or permanent.

13. The method of claim 1, wherein said subject is a human.

14. The method of claim 1, wherein said subject has not been diagnosed with fibrosis.

15. The method of claim 1, wherein said subject has not been diagnosed with cancer.

16. A method of treating atrial fibrillation or preventing persistent atrial fibrillation in a subject, comprising: administering GM-CT-01 or GR-MD-02 to said subject, wherein said administering treats or prevents atrial fibrillation in said subject.

17. A method of screening compounds, comprising:
a) administering a test compound to an ovine that exhibits a transition from paroxysmal to long-standing persistent atrial fibrillation; and
b) identifying compounds that inhibit or delay said transition.

18. The method of claim 17, w herein said paroxysmal and said self-sustained AF is induced by atrial tachypacing.

19. The method of claim 17, wherein said test compound is administered prior to a first episode of atrial fibrillation.

20. The method of claim 17, wherein said test compound is administered repeatedly.
